(19) United States
(12) Patent Application Publication
(10) Pub. No.: US 2025/0002545 A1
(43) Pub. Date: Jan. 2, 2025

(54) COMPOSITIONS AND METHODS FOR OPTOGENIC INDUCTION OF POLYPEPTIDE AGGREGATION

(71) Applicant: UNIVERSITY OF PITTSBURGH-OF THE COMMONWEALTH SYSTEM OF HIGHER EDUCATION, Pittsburgh, PA (US)

(72) Inventors: Amantha THATHIAH, Pittsburgh, PA (US); Bryan HURTLE, Pittsburgh, PA (US); Christopher James DONNELLY, Pittsburgh, PA (US); Jacob R. MANN, Pittsburgh, PA (US)

(21) Appl. No.: 18/768,994

(22) Filed: Jul. 10, 2024

Related U.S. Application Data
(63) Continuation-in-part of application No. 18/602,225, filed on Mar. 12, 2024.
Application No. 18/602,225 is a continuation of application No. 16/491,758, filed on Sep. 6, 2019, now Pat. No. 11,945,847.
(60) Provisional application No. 63/512,707, filed on Jul. 10, 2023.
(60) Provisional application No. 62/468,065, filed on Mar. 7, 2017.

(51) Int. Cl. C07K 14/47; C12N 15/62; C12N 15/79; G01N 33/50

(52) U.S. Cl. CPC C07K 14/47; C12N 15/62; C12N 15/79; G01N 33/5008; G01N 2800/28

(57) ABSTRACT

The present disclosure relates to compounds, compositions, and methods for inducing and analyzing neurodegenerative disease pathologies.

### CROSS-REFERENCE TO RELATED APPLICATIONS

This application claims the benefit of U.S. provisional patent application Ser. No. 63/512,707, filed on Jul. 10, 2023, and U.S. non-provisional patent application Ser. No. 18/602,225 filed on Mar. 12, 2024, which is a continuation of U.S. non-provisional patent application Ser. No. 16/491,758 filed Sep. 6, 2019, which is a national stage application filed under 35 U.S.C. § 371 of PCT/US2018/021335 filed Mar. 7, 2018, which claims priority to U.S. provisional patent application Ser. No. 62/468,065 filed Mar. 7, 2017, the disclosures of which are expressly incorporated herein by reference in their entireties.

### STATEMENT OF GOVERNMENT INTEREST

This invention was made with Government support under grant number AG021885 awarded by the National Institutes of Health. The U.S. Government has certain rights in this invention.

### REFERENCE TO SEQUENCE LISTING

The Sequence Listing submitted on Sep. 24, 2024, as a .XML entitled “10504-092US1_ST26.xml” created on Aug. 26, 2024, and having a size of 203,603 bytes is hereby incorporated by reference pursuant to 37 C.F.R. § 1.52(e)(5).

### FIELD

The present disclosure relates to compounds, compositions, and methods for the inducing neurodegenerative disease pathologies.

### BACKGROUND

The world is aging. By the year 2050, the proportion of individuals over the age of 60 will have doubled to 2 billion from 605 million in 2000. Unfortunately, aging is the single greatest risk factor for developing a fatal neurodegenerative disease. In turn, the number of individuals with dementias such as Alzheimer's disease (AD), Lewy Body Dementia (LBD), Frontotemporal Dementia (FTD), and movement disorders such as Parkinson's Disease (PD) and Amyotrophic Lateral Sclerosis (ALS) will significantly increase. Nearly 6.5 million individuals within the United States are currently living with one of these diseases and the associated costs are unsustainable.

In the United States, the current economic burden of AD, PD, and ALS is an estimated $241 billion dollars per year. AD and ALS/FTD patients can incur personal medical costs upwards of $100,000-$250,000 per year. For AD, it is estimated that 13.8 million individuals in the United States will have been diagnosed by 2050, up from 4.7 million in 2010, while the worldwide number of ALS cases will rise ˜31% by 2040 and no effective treatment currently exists for these disorders. Despite a variety of genetic mutations that contribute to these neurodegenerative disorders, there is no known single cause. However, despite this diversity, nearly all patients within each disease exhibit a common neuropathological hallmark in the form of intracellular protein aggregates. Animal models to recapitulate these neuropathologies currently require using either genetic mutations or grossly overexpressing proteins and these often do not mimic patient pathology. What is needed are new and improved methods for inducing neurodegenerative disease pathologies in cell lines and animal models.

The compounds, compositions, and methods disclosed herein address these and other needs.

### SUMMARY

Disclosed herein are compounds, compositions, and methods for inducing neurodegenerative disease pathologies in a cell or animal. The inventors have developed new methods for inducing neurodegenerative disease pathologies in cells and animals without the need for genetic mutations or grossly overexpressing neurodegenerative disease proteins.

In one aspect, disclosed herein is a nucleotide sequence encoding a chimeric polypeptide, comprising: a first nucleotide sequence encoding a light-induced oligomerization domain and a second nucleotide sequence encoding a low complexity domain from a neurodegenerative disease target protein.

In one aspect, disclosed herein is an expression vector encoding a chimeric polypeptide, comprising: a first nucleotide sequence encoding a light-induced oligomerization domain and a second nucleotide sequence encoding a low complexity domain from a neurodegenerative disease target protein, wherein the first nucleotide sequence is operably linked to a promoter.

In one aspect, disclosed herein is a cell comprising a nucleotide sequence encoding a chimeric polypeptide, comprising: a first nucleotide sequence encoding a light-induced oligomerization domain and a second nucleotide sequence encoding a neurodegenerative disease target protein.

In one aspect, disclosed herein is a chimeric polypeptide comprising: a light-induced oligomerization domain; and a low complexity domain from a neurodegenerative disease target protein wherein the low complexity domain from a neurodegenerative disease target protein is a Tau.

In one aspect, disclosed herein is a method of inducing a neurodegenerative disease pathology in a cell, comprising the steps:
a. introducing into the cell an expression vector encoding a chimeric polypeptide, comprising:
b. a first nucleotide sequence encoding a light-induced oligomerization domain and a second nucleotide sequence encoding a low complexity domain from a neurodegenerative disease target protein, wherein the first nucleotide sequence is operably linked to a promoter;
c. expressing the chimeric polypeptide; and
d. inducing oligomerization of the chimeric polypeptide by stimulation with blue light.

In another aspect, disclosed herein is a method of screening for an agent that modulates protein aggregation, comprising the steps:
a. introducing into a cell an expression vector encoding a chimeric polypeptide, comprising:
b. a first nucleotide sequence encoding a light-induced oligomerization domain and a second nucleotide sequence encoding a low complexity domain from a neurodegenerative disease target protein, wherein the first nucleotide sequence is operably linked to a promoter;
c. expressing the chimeric polypeptide;
d. introducing the agent into a culture media comprising the cell;
e. inducing oligomerization of the chimeric polypeptide by stimulation with blue light; and
f. determining modulation of protein aggregation by the agent.

In one embodiment, the light-induced oligomerization domain is selected from the group consisting of CRYPHR, CRY2OLIG, NcVVD, NcVVDY50W, NcLOV, VfAU1, YtvA, EL222, RsLOV, and AsLOV2. In one embodiment, the light-induced oligomerization domain is selected from the list of domains in Table 2. In one embodiment, the light-induced oligomerization domain is NcVVDY50W. In one embodiment, the light-induced oligomerization domain is CRY2OLIG. In one embodiment, the light-induced oligomerization domain is CRYPHR.

In one embodiment, the light-induced oligomerization domain is selected from the group consisting of a CRY2 PHR domain (for example, CRY2PHR, CRY2OLIG) or a light-oxygen-voltage-sensing (LOV) domain (for example, NcVVD, NcVVDY50W, VfAU1, YtvA, EL222, RsLOV, AsLOV2).

In one embodiment, the low complexity domain from a neurodegenerative disease target protein is selected from the group consisting of TDP-43, Alpha synuclein, Tau, Fus, TIA1, SOD1, Huntingtin, Ataxin 2, hnRNPA1, hnRNPA2B1, EWS RNA Binding Protein 1, and TATA box binding protein factor 15. In one embodiment, the low complexity domain from a neurodegenerative disease target protein is selected from Table 3. In one embodiment, the low complexity domain from a neurodegenerative disease target protein is TDP-43. In one embodiment, the low complexity domain from a neurodegenerative disease target protein is Alpha synuclein. In one embodiment, the low complexity domain from a neurodegenerative disease target protein is Tau.

In some embodiments, the first nucleotide sequence encodes an amino acid sequence that is at least 85%, 90% or 95% identical to SEQ ID NO:5.

In some embodiments, the first nucleotide sequence encodes an amino acid sequence comprising SEQ ID NO:103.

In some embodiments, the Tau comprises SEQ ID NO:104.

In some embodiments, the expression vector further comprises a third nucleotide sequence encoding a visualization probe binding sequence. In certain aspects, the visualization probe is a fluorescent dye. In certain aspects, the visualization probe binding sequence is a modified haloalkane dehalogenase.

In one embodiment, the cell is a mammalian cell. In one embodiment, the cell is a human cell.

In one embodiment, the blue light has a wavelength between 405 nm and 499 nm. In one embodiment, the blue light has a wavelength of about 465 nm.

### BRIEF DESCRIPTION OF THE DRAWINGS

The accompanying figures, which are incorporated in and constitute a part of this specification, illustrate several aspects described below.

FIG. 1 shows the examples of genetic causes of ALS and the common neuropathology. The left panel shows a graph of the number of ALS-causing genes on the Y axis, and the X axis indicates the year each mutation was discovered. FIG. 1, panel A shows that ALS exhibits extreme genetic heterogeneity, especially since these mutations are found in only ˜10% of ALS cases. As described in Table 1, despite these genetic causes of ALS, nearly all patients show the same neuropathology in the motor cortex and spinal cord. The right panel shows an example of ALS neuropathology by H and E staining of paraffin tissue sections from an ALS patient motor cortex. Shown are cytoplasmic aggregates of TDP-43. TDP-43 is predominantly nuclear in normal cells as represented by *. In ALS, TDP-43 is absent from the nucleus and aggregates in the cytoplasm as shown by the arrow. Although ALS is used as an example, cytoplasmic aggregate neuropathology is a common feature of many neurodegenerative diseases, sec Table 1.

FIGS. 2A-2B describe methods to generate neuropathological aggregate proteins containing an LCD/IDR/Prion-like domain with light using TDP-43 and CRY2OLIG as an example. Various protein arrangements and blue light exposure paradigms were developed to induce protein aggregation of proteins and protein fragments containing LCD/IDR/prion-like domains, promote the mislocalization of nuclear proteins and recapitulate the neuropathology of neurodegenerative diseases. TDP-43 is a predominantly nuclear protein that contains an IDR/IDD/prion-like domain, and is mislocalized and aggregated in ALS, FTD, and some AD patients, as an example. DNA sequences were engineered to generate amino acid sequences that encode for a the CRY2OLIG protein that clusters when exposed to blue light generated a fusion protein either the entire TDP-43 protein (cry2-TDP43-mCh) or just the low complexity domain (LCD/IDD/prion-like domain), which makes TDP-43 aggregation prone (Cry2-274-mCh). As a control, CRY2OLIG alone was used. All constructs were fused to a fluorescent protein called mCherry (mCh) to visualize the proteins in live cells. CRY2OLIG-mCh reversibly clusters with blue light stimulation but the CRY2OLIG-TDP-43-mCh or the truncated CRY2OLIG-274-mCh forms irreversible aggregates with specific blue light stimulation paradigms. FIG. 2A shows examples of the CRY2OLIG-TDP-43 arrangements and truncated CRY2OLIG-274 arrangements used in this work. FIG. 2B shows a model of the described technology.

FIG. 3 shows a schematic describing blue light induced oligomerization and aggregation of proteins employing the NcVVD, NcVVDY50W or NcLOV photoreceptor. The NcVVD or LOV domain have only been shown to homodimerize with blue light stimulation. A light exposure paradigm to induce oligomerization and aggregation was developed. The top panel shows a schematic of how a single acute stimulation with blue light (405-499 nm) induces the homodimerization of the LOV protein when fused to a protein of interest. The bottom panel shows that chronic stimulation with blue light promotes the homooligomerization of NcVVD or LOV fusion proteins that contain a prion-like domain/LCD/IDD.

FIGS. 4A-4E show light-induced aggregation of low-complexity domain proteins. Optogenetic TDP-43 fragments containing the low-complexity domain (LCD) undergo progressive aggregation with light stimulation. (FIGS. 4A-4B) C-terminal fragments of TDP-43 (optoRRM2+LCD/optoLCD) rapidly oligomerize after a brief pulse (8 sec, 10% laser power, 488 nm) of blue light when monitored by live confocal microscopy. These oligomers persist much longer than the Cry2 photoreceptor alone (˜10 min disassembly). (FIGS. 4C-4D) Optogenetic LCD fragments also continue to aggregate following brief light pulses, growing in size over time. (FIG. 4E) Persistent blue light stimulation of the TDP-43 LCD forms intracellular aggregates in the cells. Fluorescent recovery after photobleaching (FRAP) experiments show that persistent light LCD inclusions do not recover from FRAP indicating that these are insoluble.

FIGS. 5A-5K show chronic blue light stimulation induces optoTDP43 mislocalization and aggregation that recapitulates pathological hallmarks seen in patient CNS tissue. HEK293 cells expressing optoTDP43-mCh were exposed to 488 nm LED stimulation or darkness for up to 36 hours. (FIG. 5A-5C) Representative images showing optoTDP43 that (FIG. 5B) first experiences gradual cytoplasmic mislocalization, (FIG. 5C) which was confirmed by nuclear/cytoplasmic fractionation. (FIG. 5D) Mislocalization is followed by optoTDP43 aggregation, as measured by simultaneous chronic blue light exposure and high-throughput automated confocal microscopy, that increases in propensity with increasing light exposure. (FIG. 5E) Fluorescence recovery after photobleaching (FRAP) imaging was performed to assess dynamicity of optoTDP43 structures. Lack of fluorescence recovery shows that light-induced optoTDP43 aggregates are non-dynamic, immobile granules reminiscent of aggregated structures. (FIG. 5F) Detergent-solubility of optoTDP43 structures was assessed by subcellular fractionation to confirm aggregated state of light-induced optoTDP43 granules. (Left lanes) Non-optogenetic TDP-43 (TDP43-mCh) shows no changes in solubility with and without light treatment, while optoTDP43 (right lanes) displays a drastic shift to the insoluble fraction with chronic blue light stimulation. In addition to increased insolubility of exogenous, full-length optoTDP43 (top band), chronic blue light exposure also results in recruitment of aberrant optoTDP43 cleavage products (middle bands), as well as endogenous full-length TDP-43 and disease-relevant cleavage products (bottom bands), to the detergent-insoluble, urea-soluble fraction of cell lysates as is observed in patient tissue. (FIG. 5G) To confirm direct recruitment of non-optogenetic TDP-43 species to exogenous light-induced optoTDP43 inclusions, EGFP-tagged TDP-43 was co-expressed with optoTDP43 or the Cry2 photoreceptor-only control. In cells exposed to chronic blue light stimulation, strong co-localization is observed between optoTDP43 inclusions and EGFP-TDP43, confirming the ability of optoTDP43 inclusions to directly recruit other TDP-43 species. This recruitment appears to be dependent on TDP43:TDP43 interactions, as no co-localization is observed with Cry2-mCh puncta following blue light exposure. (FIG. 5H-5J) Immunofluorescence analysis was performed on light-induced optoTDP43 aggregates to confirm pathological hallmarks seen in patient tissue. optoTDP43 inclusions appear to be (FIG. 5H) ubiquitinated, (FIG. 5I) hyperphosphorylated, and (FIG. 5J) p62-positive, all of which have been observed with TDP-43 inclusions in patient CNS. (FIG. 5K) Automated high-throughput confocal microscopy was performed to assess the neurotoxicity of light-induced optoTDP43 inclusions. Human ReN neurons expressing TDP43-mCh or optoTDP43 were exposed to chronic blue light stimulation and viability was simultaneously monitored by longitudinal imaging. Neurons expressing non-optogenetic TDP43-mCh showed no significant decrease in survival with or without blue light exposure. However, neurons expressing optoTDP43 that were exposed to blue light stimulation display significantly decreased viability over time, suggesting optoTDP43 inclusions are neurotoxic. *=p<0.05, **=p<0.01

FIGS. 6A-6E show chronic stimulation of CRY2OLIG α-synuclein or a-synuclein LOV induces clustering and aggregation. Chronic stimulation of a CRY2-asyn-mCh was tested to induce clustering or the α-synuclein protein. FIG. 6A shows the chronic stimulation paradigm and FIG. 6B shows representative images of a-synuclein clustering with light over time along with quantification of clustering in FIG. 6C. These data indicate that this optogenetic system can be employed to induce the clustering and aggregation of multiple neurodegenerative disease proteins that are prone to aggregation, that is, contain prion-like domains/LCD/IDDs. FIG. 6D shows that a-synuclein fused to the LOV (dimerizing photoreceptor) forms intracellular clusters of alpha synuclein. FIG. 6E shows that light induced a-synuclein LOV aggregates exhibit pathological hallmarks of synucleinopathies including phosphorylation at serine 129 and p62 positivity.

FIGS. 7A-7D show Cry2-Tau fusion protein shows pathological markers for various tauopathies. (FIGS. 7A-7C) HEK293 cells expressing Cry2 photoreceptor-alone or Cry2-Tau fusion proteins were exposed to chronic blue light stimulation (16 hours, 488 nm, 10 mW). Cry2-Tau-expressing cells show fibril-like aggregates that co-stain with phospho-Tau antibodies AT8 (FIG. 7A) and PHF1 (FIG. 7B), as well as the pathological conformation-dependent Tau antibody MC1 (FIG. 7C). (FIG. 7D) Summary showing the various optogenetic Tau constructs that co-localize with pathological Tau antibodies after light-induction of neurofibrillary tangle formation.

FIGS. 8A-8E show LOV-Tau fusion proteins also show pathological markers for various tauopathies and are insoluble. (FIG. 8A-FIG. 8B) HEK293 cells expressing VVD-Tau or VfAU-Tau fusion proteins were exposed to chronic blue light stimulation (16 hours, 488 nm, 10 mW). Both fusion proteins co-stain with phospho-Tau antibodies AT8 and PHF1, and the pathological conformation-dependent Tau antibody MC1. (FIG. 8C) Differentiated MAP2+ human ReN neurons expressing VVD-Tau also shows neurofibrillary tangle formation following chronic light treatment that co-localizes with AT8. (FIG. 8D) Fluorescence recovery after photobleaching analysis was performed on light-induced tangles and shows a lack of fluorescence recovery, indicating a non-dynamic, immobile structure. (FIG. 8E) Urea extraction was next performed to confirm insoluble tau species with light treatment in HEK293 cells expressing VVD-Tau. Cells exposed to chronic light show increased levels of soluble and insoluble 150 kDa tau species which is present in Alzheimer's Disease and Frontotemporal Dementia patient tissue.

FIGS. 9A-9B show the aggregation potential of CRY2 or VVD fusion proteins based on the protein arrangement and light stimulation paradigm combination. Studies indicate that the ability of Cry2 or VVD photoreceptor to stimulate clustering and activation are dependent on the protein arrangement as well as the light stimulation paradigm. FIG. 9A shows that a TDP-43-CRY2OLIG-mCh protein mislocalizes but does not exhibit the ability to aggregate as the CRY2OLIG-TDP-43-mCh arrangement does. Similarly, FIG. 9B shows that the mCh-aSyn-NvVVDY50W does not have the ability to aggregate. This data shows that the protein arrangement and light stimulation paradigms are important for inducing neuropathological protein aggregates as observed in the CNS tissue of patients with neurodegenerative disease.

FIGS. 10A-10B show that blue light induced TDP-43 aggregates or truncated LCD/IDR/prion-like domain aggregates are toxic to cells. FIG. 10A shows that acute stimulation of HEK cells expressing CRY2OLIG-TDP274-mCh can induce the formation of pathological aggregates within 1 hour. These aggregates can grow in size and become toxic over time. FIG. 10B is a schematic of a screening pipeline to use the induction of neurodegenerative disease pathology to identify therapeutic compounds that rescue toxicity and prevent or remove the formation of the induced neurodegenerative disease pathology.

FIGS. 11A-11J show that OptoTAU displays superior light selectivity and control of tau aggregation. (A-B) Representative immunoblot images (A) and densitometric analysis (B) of total protein homogenates from HEK293 cells following expression of tau alone (negative control), VVD-tau, tau-VVD, Cry2-tau, and tau-Cry2. A lower (upper panel) and higher (middle panel) exposure are shown for the representation of monomeric and HMW tau (A). (C) Schematic of the optoTAU time-course experimental paradigm. (D-F) Representative immunoblot images (D) and densitometric analysis (E, F) of the temporal accumulation of soluble HMW and insoluble optoTAU (D; upper panel) and monomeric optoTAU (D; middle panel) with or without (24 h DARK) light stimulation for 1, 4, and 24 hours. (G-J) Representative immunoblot images and densitometric analysis of soluble (G, H) and insoluble (I, J) optoTAU in HEK293 cells following treatment with 50, 250, or 1000 ng/ml of Dox with or without light stimulation. The pan-tau antibody Tau5 was used for immunoblot detection of tau. Densitometric analysis of HMW tau was normalized to monomeric tau (C). OptoTAU monomer levels are normalized to total protein (Ponceau S; lower panels) (A, D, G). Densitometric analysis of soluble HMW optoTAU (>100 kDa) was normalized to monomeric optoTAU (<75 kDa). (D, G) and total insoluble tau represents the monomer+HMW tau (D, I). Each data point represents the average of two technical replicates for 3-4 independent experiments.

FIGS. 12A-12K show that OptoTAU permits the temporal live cell visualization of the progressive transition in tau solubility and forms persistent tau aggregates. (A) Representative immunocytochemical analysis of soluble (P1h; top panel), insoluble (P18h; middle panel), and the overlay of both dyes. Scale bar=10 μm. (B-C) Average soluble (B) and insoluble (C) fluorescence per cell. Data is an average of n=3 independent experiments (90-125 cells from n=6 fields of view per condition). (D-G) Twenty four hours after OptoTAU transfection, doxycycline was added to induce expression, followed by a 24 hour culture in the dark and then four different light-stimulation paradigms. Representative immunoblot images and densitometric analysis of the accumulation of soluble HMW (D, E) and insoluble (F, G) optoTAU in HEK293 cells following those four different light-stimulation paradigms from left to right in each figure: (1) 24 h dark alone (negative control), (2) 12 h light exposure followed by 12 h under dark condition (persistence test), (3) 12 h dark condition followed by 12 h light exposure, and (4) 24 h light exposure (positive control). The pan-tau antibody Tau5 was used for immunoblot detection of tau. HMW optoTAU images (D, F) are higher exposure to clearly show HMW tau detection. Soluble optoTAU monomer levels are normalized to total protein (Ponceau S; lower panels) (D). Densitometric analysis of soluble HMW optoTAU (>100 kDa) was normalized to monomeric optoTAU (<75 kDa) (D) and total insoluble tau represents the monomer+HMW tau (F). Each data point represents the average of two technical replicates for 3-4 independent experiments. (H) Schematic of the experimental paradigm to visualize the progressive and persistent accumulation of optoTAU-Halo with the AggFluor probes P1h (soluble oligomers) and P18h (insoluble aggregates). (I) Representative live-cell images of soluble (middle row), insoluble (bottom row), and the overlay of both dyes (top row) in cells prior to light stimulation (I.A), following dark conditions alone for 24 h (I.B), or following light stimulation for 24 h alone (I.C) and subsequent dark conditions for 24 h (I.D). (J-K) Average soluble (J) and insoluble (K) fluorescence per cell. Soluble and insoluble fluorescence was normalized to total nuclei number per field of view (n=3 independent experiments; 70-120 cells from n=2 fields of view/condition). Ordinary One-Way ANOVA analysis followed by Tukey's multiple comparisons. Mean±SEM (error bars). ***=p<0.0001, **=p<0.001, *=p<0.05, ns=not significant.

FIGS. 13A-13J shows that OptoTAU recapitulates pathophysiological features of tau aggregation. (A) Schematic of the pro- and anti-aggregation mutations in optoTAU. (B-E) Representative immunoblot images (B, D) and densitometric analysis (C, E) of the accumulation of soluble HMW (B, C) and insoluble (D, E) optoTAU with or without light stimulation in cells expressing optoTAU(WT), optoTAU(K280; PRO), or optoTAU(K280, I277P,I308P; ANTI). (F) Schematic of optoTAU isoforms. (G-J) Representative immunoblot images (G,I) and densitometric analysis (H,J) of soluble HMW (G,H) and insoluble (I,J) optoTAU with or without light stimulation in cells expressing 0N4R, 1N4R, or 2N4R optoTAU. The pan-tau antibody Tau5 was used for immunoblot detection of tau. HMW optoTAU images (B, D, G, I) are higher exposure to clearly show HMW tau detection. Soluble optoTAU monomer levels are normalized to total protein (Ponceau S; lower panels) (B, G). Densitometric analysis of soluble HMW optoTAU (>100 kDa) was normalized to monomeric optoTAU (<75 kDa) (C, H) and total insoluble tau represents the monomer+HMW tau (D, I). Each data point represents the average of two technical replicates for 3-4 independent experiments. Ordinary One-Way ANOVA analysis followed by Tukey's multiple comparisons. Mean±SEM (error bars). ***=p<0.0001, **=p<0.001, *=p<0.05, ns=not significant.

FIGS. 14A-14K shows that OptoTAU-Halo recruits endogenous tau into tau aggregates and forms amyloid structure in human neurons. (A) Schematic diagram of the experimental paradigm to detect optoTAU-Halo amyloid structure in human neurons. (B) Representative immunocytochemical analysis of Amytracker™ alone (lower panels) and V5/Amytracker™ overlay (upper panels). (C) Quantification of the total percent area occupied by Amytracker™ relative to optoTAU-Halo (V5). (D) LDH cytotoxicity assay using culture supernatants from VVD-V5 and optoTAU-Halo expressing human neurons. (E) Schematic of the experimental paradigm using the “always-on” Oregon Green HaloTag dye to investigate optoTAU-Halo expression and colocalization with endogenous 3R tau in human neurons. (F) Representative immunocytochemical analysis of optoTAU-Halo (Oregon green), endogenous 3R tau, and Amytracker™. Co-localization of optoTAU-Halo and Amytracker™ (top right), optoTAU-Halo and 3R tau (middle right), and 3R tau and Amytracker (lower right). (G) Schematic of the live cell imaging experimental timeline in human neurons. (H) Representative immunocytochemical analysis of soluble (P1h; upper panels), insoluble (P18h; middle panels), and the overlay of both dyes (lower panels) with or without W-MINK (10 μM) treatment. (I-K) Average soluble (I) and insoluble (J) fluorescence normalized to nuclei count for Control and W-MINK treated cells. Data points are an average of n=3 independent experiments (90-125 cells from n=6 fields of view per condition). (K) Total nuclei count with or without W-MINK treatment. Scale bar=10 μm. Ratio paired t-test (I-K). Ordinary One-Way ANOVA (C, D) followed by Tukey's multiple comparisons (C, D). All mean±SEM (error bars). ****=p<0.00001, ***=p<0.0001, **=p<0.001, *=p<0.05.

FIGS. 15A-15J show that OptoTAU-Halo is a light-specific optogenetic model of tau aggregation. (A) Schematic of the experimental paradigm to establish optoTAU-Halo as an optogenetic model of tau aggregation in a HEK293 cell line that stably expresses optoTAU-Halo. (B-D) Representative immunoblot images (B) and densitometric analysis (C, D) of the tau monomer and soluble HMW tau after loading three different volumes of each sample from light-exposed optoTAU-Halo samples. (E) Densitometric analysis of total soluble optoTAU-Halo (HMW+Monomer) in dark and light-exposed soluble fractions. (F) Densitometric analysis of total optoTAU-Halo monomer levels in soluble fractions (tau monomer alone) in dark and light-exposed soluble fractions. (G) Densitometric analysis of total HMW optoTAU-Halo levels in soluble fractions (HMW tau alone) in dark and light-exposed soluble fractions. (H) Densitometric analysis of soluble HMW optoTAU-Halo relative to optoTAU-Halo monomer levels in soluble fractions (HMW/monomer tau ratio) in dark and light-exposed soluble fractions. (I) Representative immunoblot images of insoluble optoTAU-Halo with or without light stimulation. (J) Densitometric analysis of total insoluble optoTAU-Halo with or without light exposure. Each data point represents one technical replicate for n=4 independent experiments. The pan-tau antibody Tau5 was used for immunoblot detection of tau. For (E-H, J), unpaired t-test and for (C, D), Ordinary One-Way ANOVA analysis followed by Tukey's multiple comparisons. Mean±SEM (error bars). ****=p<0.00001, ***=p<0.0001, **=p<0.001, *=p<0.01, ns=not significant.

FIGS. 16A-16F show the establishment of optoTAU-Halo as a model of tau aggregation in human neurons and recruitment of endogenous tau into optoTAU-Halo aggregates. (A) Schematic of the ReNcell NPC differentiation protocol and representative immunocytochemical analysis of MAP2+ (neuronal marker) and GFAP+ (astrocytic marker) cells following differentiation for 25 days. (B-E) Representative immunoblot images of VVD-V5 (negative control) and optoTAU-Halo-expressing neurons with or without light stimulation. Soluble fractions were probed with a pan tau antibody (Tau5) (B), a V5 antibody to specifically detect optoTAU-Halo alone or the VVD-V5 negative control, or with a 3R tau-isoform specific antibody to detect endogenous tau (E). Insoluble fractions were probed with the V5 antibody (D). (F) Representative immunocytochemical analysis of the Dox (−) and Dox (+) conditions with the Oregon green HaloTag dye alone (left panels), endogenous 3R tau alone (middle panels), and the overlay (right panels) in optoTAU-Halo expressing neurons. Scale bar=10 μm.

### DETAILED DESCRIPTION

Disclosed herein are compounds, compositions, and methods for inducing neurodegenerative disease pathologies in a cell or animal. The inventors have developed new methods for inducing neurodegenerative disease pathologies in cells and animals without the need for genetic mutations or grossly overexpressing neurodegenerative disease proteins.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

The following definitions are provided for the full understanding of terms used in this specification.

### Terminology

As used herein, the article “a,” “an,” and “the” means “at least one,” unless the context in which the article is used clearly indicates otherwise.

The term “about” as used herein when referring to a measurable value such as an amount, a percentage, and the like, is meant to encompass variations of ±20%, ±10%, ±5%, or ±1% from the measurable value.

As used herein, “corresponding mutation” refers to a mutation that provides the same or similar effect on polypeptide functionality. For example, mutations that correspond to the I74V and I85V mutations of VVD from Neurospora crassa also allow for improved light induced oligomerization, or aggregation, as a result of enhanced dimerization efficiency and/or faster switch-off kinetics. In some embodiments, the corresponding mutations are conservative amino acid substitutions.

As used herein, the terms “may,” “optionally,” and “may optionally” are used interchangeably and are meant to include cases in which the condition occurs as well as cases in which the condition does not occur.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The terms “polynucleotide”, “nucleotide sequence”, and “nucleic acid sequence” are used interchangeably herein and refer to a single or double stranded polymer composed of nucleotide monomers.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill in the art will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (sec Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “variant” or “derivative” as used herein refers to an amino acid sequence derived from the amino acid sequence of the parent protein having one or more amino acid substitutions, insertions, and/or deletions.

### Chimeric Constructs

In one aspect, disclosed herein is a nucleotide sequence encoding a chimeric polypeptide, comprising: a first nucleotide sequence encoding a light-induced oligomerization domain and a second nucleotide sequence encoding a low complexity domain from a neurodegenerative disease target protein.

In one aspect, disclosed herein is an expression vector encoding a chimeric polypeptide, comprising: a first nucleotide sequence encoding a light-induced oligomerization domain and a second nucleotide sequence encoding a low complexity domain from a neurodegenerative disease target protein, wherein the first nucleotide sequence is operably linked to a promoter.

In some embodiments, the expression vector encoding a chimeric polypeptide is comprised in a plasmid or in a virus or viral vector. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector encoding a chimeric polypeptide is a naked DNA or is comprised in a nanoparticle (e.g., liposomal vesicle, porous silicon nanoparticle, gold-DNA conjugate particle, polyethylenimine polymer particle, cationic peptides, etc.).

The expression vectors disclosed herein are, in some embodiments, capable of inducing a neurodegenerative disease pathology in a cell (e.g., inducing aggregation of a protein) without substantially altering the expression level of a protein involved in the neurodegenerative disease pathology. For example and without limitation, the expression vector can induce aggregation of a protein having a low complexity domain from a neurodegenerative disease target protein without substantially increasing or decreasing the expression level of an endogenous target protein which comprises the same low complexity domain. As such, a cell comprising a herein disclosed nucleotide sequence encoding a chimeric polypeptide can have substantially unchanged expression levels of an endogenous neurodegenerative disease target protein comprising a low complexity domain, as compared to a cell of the same cell type which does not comprise the nucleotide sequence encoding a chimeric polypeptide. The term “substantially unchanged expression levels,” as used herein, refers to a change in expression level, if any, to a degree not known or not suspected to cause or be associated with inducing a neurodegenerative disease pathology in a cell.

The expression vectors disclosed herein are, in some embodiments, capable of inducing a neurodegenerative disease pathology in a cell (e.g., inducing aggregation of a protein) using a wild-type form of a low complexity domain from a neurodegenerative disease target protein. Thus, in some embodiments, the low complexity domain from a neurodegenerative disease target protein does not include a mutation which differs from the wild-type sequence and which is known or suspected to cause or be associated with inducing a neurodegenerative disease pathology in a cell. For example and without limitation, the expression vector can comprise a low complexity domain from a wild-type TDP-43 protein which does not contain a mutation such as Q331K, known to cause or be associated with ALS.

In one aspect, disclosed herein is a cell comprising a nucleotide sequence encoding a chimeric polypeptide, comprising: a first nucleotide sequence encoding a light-induced oligomerization domain and a second nucleotide sequence encoding a neurodegenerative disease target protein.

In some embodiments, the cell is a cell which can be affected by a neurodegenerative disease. For example, the cell can be a glial cell or a neuronal cell.

In one aspect, disclosed herein is a chimeric polypeptide comprising: a light-induced oligomerization domain; and a low complexity domain from a neurodegenerative disease target protein.

In one embodiment, the light-induced oligomerization domain is selected from the group consisting of CRYPHR, CRY2OLIG, NcVVD, NcVVDY50W, and NcLOV. In one embodiment, the light-induced oligomerization domain is selected from the group consisting of CRYPHR, CRY2OLIG, NcVVD, NcVVDY50W, NcLOV, VfAU1, YtvA, EL222, RsLOV, and AsLOV2. In one embodiment, the light-induced oligomerization domain is selected from the list of domains in Table 2. In one embodiment, the light-induced oligomerization domain is selected from a variant of CRYPHR, CRY2OLIG, NcVVD, NcVVDY50W, NcLOV, VfAU1, YtvA, EL222, RsLOV, or AsLOV2. In one embodiment, the light-induced oligomerization domain is selected from a fragment of CRYPHR, CRY2OLIG, NcVVD, NcVVDY50W, NcLOV, VfAU1, YtvA, EL222, RsLOV, or AsLOV2.

In one embodiment, the light-induced oligomerization domain is NcVVDY50W. In one embodiment, the light-induced oligomerization domain is CRY2OLIG. In one embodiment, the light-induced oligomerization domain is CRYPHR. In one embodiment, the light-induced oligomerization domain comprises a LOV domain from the VVD protein. In one embodiment, the light-induced oligomerization domain comprises a LOV domain from the LOV protein. In one embodiment, the light-induced oligomerization domain comprises a PHR domain. In one embodiment, the light-induced oligomerization domain comprises a PHR domain, from the CRY2 protein. In one embodiment, the light-induced oligomerization domain is VfAU1. In one embodiment, the light-induced oligomerization domain is YtvA. In one embodiment, the light-induced oligomerization domain is EL222. In one embodiment, the light-induced oligomerization domain is RsLOV. In one embodiment, the light-induced oligomerization domain is AsLOV2.

In one embodiment, the light-induced oligomerization domain is at least 90% identical to CRYPHR. In one embodiment, the light-induced oligomerization domain is at least 90% identical to NcVVD. In one embodiment, the light-induced oligomerization domain is at least 90% identical to NcVVDY50W. In one embodiment, the light-induced oligomerization domain is at least 90% identical to NcLOV. In one embodiment, the light-induced oligomerization domain is at least 90% identical to CRY2OLIG. In one embodiment, the light-induced oligomerization domain is at least 90% identical to VfAU1. In one embodiment, the light-induced oligomerization domain is at least 90% identical to YtvA. In one embodiment, the light-induced oligomerization domain is at least 90% identical to EL222. In one embodiment, the light-induced oligomerization domain is at least 90% identical to RsLOV. In one embodiment, the light-induced oligomerization domain is at least 90% identical to AsLOV2.

In some embodiments, the first nucleotide sequence can comprise a nucleotide sequence which encodes a light-induced oligomerization domain selected from the group consisting of CRYPHR, CRY2OLIG, NcVVD, NcVVDY50W, NcLOV, and VfAU1LOV.

In some embodiments, the first nucleotide sequence can comprise a nucleotide sequence which encodes an amino acid sequence that is at least 70% identical to SEQ ID NO:1, SEQ ID NO:2, SEQ ID NO:3, SEQ ID NO:4, SEQ ID NO:5, SEQ ID NO:92, SEQ ID NO:93, SEQ ID NO:99, SEQ ID NO:100, SEQ ID NO:101, or SEQ ID NO:102. In some embodiments, the first nucleotide sequence can encode an amino acid sequence which is at least 75% identical, at least 80% identical, at least 85% identical, at least 90% identical, at least 95% identical, or at least 99% identical to SEQ ID NO:1, SEQ ID NO:2, SEQ ID NO:3, SEQ ID NO:4, SEQ ID NO:5, SEQ ID NO:92, SEQ ID NO:93, SEQ ID NO:99, SEQ ID NO:100, SEQ ID NO:101, or SEQ ID NO:102. In some embodiments, the first nucleotide sequence can comprise SEQ ID NO:1, SEQ ID NO:2, SEQ ID NO:3, SEQ ID NO:4, SEQ ID NO:5, SEQ ID NO:92, SEQ ID NO:93, SEQ ID NO:99, SEQ ID NO:100, SEQ ID NO:101, or SEQ ID NO:102. In some embodiments, the first nucleotide sequence can comprise SEQ ID NO:94. The nucleotide sequence can be that of the wild type nucleic acid sequence encoding an amino acid sequence disclosed herein. In some embodiments, the nucleotide sequence is modified from the wild type sequence, but due to the degeneracy of the genetic code, can still encode for the same amino acid sequence. In some embodiments, the nucleotide sequence is a variant of one of the sequences disclosed herein (or encodes of variant protein sequence). In some embodiments, the nucleotide sequence is a fragment of one of the nucleic acids herein, or encodes a fragment of one of the amino acids disclosed herein. In some embodiments, the nucleotide sequence is codon optimized (for example, to improve expression).

In one embodiment, the light-induced oligomerization domain is selected from the group consisting of a CRY2 PHR domain (for example, CRY2PHR, CRY2OLIG) or a light-oxygen-voltage-sensing (LOV) domain (for example, NcVVD, NcVVDY50W, VfAU1, YtvA, EL222, RsLOV, AsLOV2).

In one embodiment, the low complexity domain from a neurodegenerative disease target protein is selected from the group consisting of TDP-43, Alpha synuclein, Tau, Fus, TIA1, SOD1, Huntingtin, Ataxin 2, and hnRNPA2B1. In one embodiment, the low complexity domain from a neurodegenerative disease target protein is selected from the group consisting of TDP-43, Alpha synuclein, Tau, Fus, TIA1, SOD1, Huntingtin, Ataxin 2, hnRNPA1, and hnRNPA2B1. In one embodiment, the low complexity domain from a neurodegenerative disease target protein is selected from the group consisting of TDP-43, Alpha synuclein, Tau, Fus, TIA1, SOD1, Huntingtin, Ataxin 2, hnRNPA1, hnRNPA2B1, EWS RNA Binding Protein 1, and TATA box binding protein factor 15. In one embodiment, the low complexity domain from a neurodegenerative disease target protein is selected from a variant of TDP-43, Alpha synuclein, Tau, TIA1, SOD1, Huntingtin, Ataxin 2, hnRNPA1, hnRNPA2B1, EWS RNA Binding Protein 1, or TATA box binding protein factor 15. In one embodiment, the low complexity domain from a neurodegenerative disease target protein is selected from a fragment of TDP-43, Alpha synuclein, Tau, TIA1, SOD1, Huntingtin, Ataxin 2, hnRNPA1, hnRNPA2B1, EWS RNA Binding Protein 1, or TATA box binding protein factor 15. In one embodiment, the fragment of TDP-43, Alpha synuclein, Tau, TIA1, SOD1, Huntingtin, Ataxin 2, hnRNPA1, hnRNPA2B1, EWS RNA Binding Protein 1, or TATA box binding protein factor 15 comprises a low complexity domain (or fragment thereof) within each neurodegenerative disease target protein.

In one embodiment, the low complexity domain from a neurodegenerative disease target protein is selected from Table 3. In one embodiment, the low complexity domain is from any neurodegenerative disease target protein that aggregates in neurodegenerative diseases.

In one embodiment, the low complexity domain from a neurodegenerative disease target protein is selected from the group consisting of an amino acid sequence with at least 90% identity to TDP-43, Alpha synuclein, Tau, Fus, TIA1, SOD1, Huntingtin, Ataxin 2, hnRNPA1, hnRNPA2B1, EWS RNA Binding Protein 1, and TATA box binding protein factor 15. In one embodiment, the low complexity domain from a neurodegenerative disease target protein is selected from the group consisting of an amino acid sequence with at least 90% identity to a low complexity domain from a neurodegenerative disease target protein is selected from Table 3. In one embodiment, the low complexity domain from a neurodegenerative disease target protein is selected from an orthologue of the group consisting of TDP-43, Alpha synuclein, Tau, Fus, TIA1, SOD1, Huntingtin, Ataxin 2, hnRNPA1, hnRNPA2B1, EWS RNA Binding Protein 1, and TATA box binding protein factor 15.

In one embodiment, the low complexity domain from a neurodegenerative disease target protein is selected from the group consisting of an amino acid sequence with at least 60% (for example, at least 60%, at least 70%, at least 80%, at least 90%, at least 95%) identity to TDP-43, Alpha synuclein, Tau, Fus, TIA1, SOD1, Huntingtin, Ataxin 2, hnRNPA1, hnRNPA2B1, EWS RNA Binding Protein 1, and TATA box binding protein factor 15 (or a fragment thereof).

In one embodiment, the low complexity domain from a neurodegenerative disease target protein is TDP-43. In one embodiment, the low complexity domain from a neurodegenerative disease target protein is Alpha synuclein. In one embodiment, the low complexity domain from a neurodegenerative disease target protein is Tau. In one embodiment, the low complexity domain from a neurodegenerative disease target protein is Fus. In one embodiment, the low complexity domain from a neurodegenerative disease target protein is TIA1. In one embodiment, the low complexity domain from a neurodegenerative disease target protein is SOD1. In one embodiment, the low complexity domain from a neurodegenerative disease target protein is Huntingtin. In one embodiment, the low complexity domain from a neurodegenerative disease target protein is Ataxin 2. In one embodiment, the low complexity domain from a neurodegenerative disease target protein is hnRNPA1. In one embodiment, the low complexity domain from a neurodegenerative disease target protein is hnRNPA2B1. In one embodiment, the low complexity domain from a neurodegenerative disease target protein is EWS RNA Binding Protein 1. In one embodiment, the low complexity domain from a neurodegenerative disease target protein is TATA box binding protein factor 15.

In one embodiment, a VVD light-induced oligomerization domain is fused to a low complexity domain of TDP-43. In one embodiment, a VVD light-induced oligomerization is fused to full length TDP-43 (comprising a low complexity domain). In one embodiment, a light-induced oligomerization domain is fused to a low complexity domain from any neurodegenerative disease target protein that aggregates in neurodegenerative diseases.

In one embodiment, a light-induced oligomerization domain is fused to a low complexity domain of TDP-43, wherein the sequence of the low complexity domain of TDP-43 comprises SEQ ID NO:95, SEQ ID NO:96, SEQ ID NO:97, or SEQ ID NO:98 (or a fragment thereof).

In one embodiment, the nucleotide sequence encoding the chimeric polypeptide may further comprise a third nucleotide sequence encoding a reporter protein such as a fluorescent protein (to allow visualization of the protein aggregates by fluorescence). In one embodiment, the fluorescent protein is mCherry (mCh). In some embodiments, the fluorescent protein is GFP or YFP. In some embodiments, the first nucleotide sequence can encode a light-induced oligomerization domain comprising a PHR domain of the Arabidopsis Cryptochrome 2 protein (e.g., CRYPHR). In some embodiments, the light-induced oligomerization domain can comprise a wild-type CRYPHR amino acid sequence as disclosed, for example, in SEQ ID NO:1, or optionally, can comprise a mutated CRYPHR amino acid sequence as disclosed, for example, in SEQ ID NO:2. In some embodiments, a mutated CRYPHR amino acid sequence can comprise an E490G mutation, which can increase efficiency of clustering upon blue light stimulation as compared to a wild-type CRYPHR amino acid sequence. In some embodiments, the first nucleotide sequence can encode a light-induced oligomerization domain, wherein the light-induced oligomerization domain comprises a polypeptide sequence which is at least 70% identical to SEQ ID NO:1 or SEQ ID NO:2. In some embodiments, the first nucleotide sequence can encode a light-induced oligomerization domain, wherein the light-induced oligomerization domain comprises a polypeptide sequence which is at least 75% identical, at least 80% identical, at least 85% identical, at least 90% identical, at least 95% identical, or at least 99% identical to SEQ ID NO:1 or SEQ ID NO:2. In some embodiments, the first nucleotide sequence can encode a light-induced oligomerization domain comprising the polypeptide sequence of SEQ ID NO:1 or SEQ ID NO:2.

In some embodiments, the first nucleotide sequence can encode a light-induced oligomerization domain comprising Light-Oxygen-Voltage-Sensing Domain (LOV) from Neurospora Vivid protein (e.g., LOV, NcVVD, NcVVDY50W, NcLOV, VfAU1, YtvA, EL222, RsLOV, and/or AsLOV2). In some embodiments, the light-induced oligomerization domain can comprise a wild-type LOV amino acid sequence as disclosed, for example, in SEQ ID NO:3,SEQ ID NO:4, SEQ ID NO:92, SEQ ID NO:93, SEQ ID NO:99, SEQ ID NO:100, SEQ ID NO:101, or SEQ ID NO:102, or optionally, can comprise a mutated LOV amino acid sequence as disclosed, for example, in SEQ ID NO:5. In some embodiments, a mutated LOV amino acid sequence can comprise a Y50W mutation, which can reduce the rate of dissipation from a dimerized state as compared to a wild-type LOV amino acid sequence. In some embodiments, the first nucleotide sequence can encode a light-induced oligomerization domain, wherein the light-induced oligomerization domain comprises a polypeptide sequence which is at least 70% identical to SEQ ID NO:3, SEQ ID NO:4, SEQ ID NO:5, SEQ ID NO:92, SEQ ID NO:93, SEQ ID NO:99, SEQ ID NO:100, SEQ ID NO:101, or SEQ ID NO:102. In some embodiments, the first nucleotide sequence can encode a light-induced oligomerization domain, wherein the light-induced oligomerization domain comprises a polypeptide sequence which is at least 75% identical, at least 80% identical, at least 85% identical, at least 90% identical, at least 95% identical, or at least 99% identical to SEQ ID NO:3, SEQ ID NO:4, SEQ ID NO:5, SEQ ID NO:92, SEQ ID NO:93, SEQ ID NO:99, SEQ ID NO:100, SEQ ID NO:101, or SEQ ID NO:102. In some embodiments, the first nucleotide sequence can encode a light-induced oligomerization domain comprising the polypeptide sequence of SEQ ID NO:3, SEQ ID NO:4, SEQ ID NO:5, SEQ ID NO:92, SEQ ID NO:93, SEQ ID NO:99, SEQ ID NO:100, SEQ ID NO:101, or SEQ ID NO:102.

In some embodiments, the first nucleotide sequence can encode a light-induced oligomerization domain comprising a PHR domain of the Arabidopsis Cryptochrome 2 protein (e.g., CRYPHR), and the second nucleotide sequence can encode a low complexity domain from a neurodegenerative disease target protein. In some embodiments, the first nucleotide sequence can encode a light-induced oligomerization domain comprising a PHR domain of the Arabidopsis Cryptochrome 2 protein (e.g., CRYPHR), and the second nucleotide sequence can encode a low complexity domain from a neurodegenerative disease target protein comprising TDP-43. In some embodiments, the second nucleotide sequence can encode a low complexity domain from a neurodegenerative disease target protein comprising full-length TDP-43 (e.g., SEQ ID NO:6 or SEQ ID NO:7). In some embodiments, the second nucleotide sequence can encode a low complexity domain from a neurodegenerative disease target protein comprising truncated TDP-43 (e.g., SEQ ID NO:8, SEQ ID NO: 9, SEQ ID NO:10, SEQ ID NO:11, SEQ ID NO:12, or SEQ ID NO:13). In some embodiments, a truncated TDP-43 comprises or consists of amino acids 105-414, 191-414, or 274-414 of the full-length TDP-43 amino acid sequence. Thus, in some embodiments, the first nucleotide sequence can encode a light-induced oligomerization domain comprising CRYPHR, and the second nucleotide sequence can encode a low complexity domain from a neurodegenerative disease target protein comprising full-length or truncated TDP-43, wherein the first nucleotide sequence and the second nucleotide sequence encode a chimeric polypeptide sequence which is at least 75% identical, at least 80% identical, at least 85% identical, at least 90% identical, at least 95% identical, or at least 99% identical to SEQ ID NO:6, SEQ ID NO:7, SEQ ID NO:8, SEQ ID NO:9, SEQ ID NO:10, SEQ ID NO:11, SEQ ID NO:12, or SEQ ID NO:13. In some embodiments, the first nucleotide sequence and the second nucleotide sequence encode a chimeric polypeptide sequence according to SEQ ID NO:6, SEQ ID NO:7, SEQ ID NO:8, SEQ ID NO:9, SEQ ID NO:10, SEQ ID NO:11, SEQ ID NO:12, or SEQ ID NO:13. In some embodiments, SEQ ID NO:6 is selected. In some embodiments, SEQ ID NO:11 is selected.

In some embodiments, a nucleotide sequence encoding a chimeric polypeptide comprising a first nucleotide sequence and a second nucleotide sequence can further contain a third nucleotide sequence encoding a reporter protein or fragment thereof (e.g., a fluorescent protein such as mCherry, also referred to as mCH). Thus, in some embodiments, a first nucleotide sequence can encode a light-induced oligomerization domain comprising CRYPHR, a second nucleotide sequence can encode a low complexity domain from a neurodegenerative disease target protein comprising full-length or truncated TDP-43, and a third nucleotide sequence can encode a mCherry protein, wherein the first, second, and third nucleotide sequences encode a chimeric polypeptide sequence which is at least 75% identical, at least 80% identical, at least 85% identical, at least 90% identical, at least 95% identical, or at least 99% identical to SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25. In some embodiments, the first, second, and third nucleotide sequences encode a chimeric polypeptide sequence according to SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25. In some embodiments, SEQ ID NO:14 is selected. In some embodiments, SEQ ID NO:23 is selected.

In some embodiments, the first nucleotide sequence can encode a light-induced oligomerization domain comprising a LOV photoreceptor domain (e.g., NcVVD, NcVVDY50W, NcLOV, VfAU1, YtvA, EL222, RsLOV, and AsLOV2), and the second nucleotide sequence can encode a low complexity domain from a neurodegenerative disease target protein. In some embodiments, the first nucleotide sequence can encode a light-induced oligomerization domain comprising a LOV photoreceptor domain (e.g., NcVVD, NcVVDY50W, NcLOV, VfAU1, YtvA, EL222, RsLOV, and AsLOV2), and the second nucleotide sequence can encode a low complexity domain from a neurodegenerative disease target protein comprising TDP-43. In some embodiments, the second nucleotide sequence can encode a low complexity domain from a neurodegenerative disease target protein comprising full-length TDP-43 (e.g., SEQ ID NO:26 or SEQ ID NO:27). In some embodiments, the second nucleotide sequence can encode a low complexity domain from a neurodegenerative disease target protein comprising truncated TDP-43 (e.g., SEQ ID NO:28, SEQ ID NO:29, SEQ ID NO:30, SEQ ID NO:31, SEQ ID NO:32, or SEQ ID NO:33). In some embodiments, a truncated TDP-43 consists of amino acids 105-414, 191-414, or 274-414 of the full-length TDP-43 amino acid sequence. Thus, in some embodiments, the first nucleotide sequence can encode a light-induced oligomerization domain comprising NcVVDY50W, and the second nucleotide sequence can encode a low complexity domain from a neurodegenerative disease target protein comprising full-length or truncated TDP-43, wherein the first nucleotide sequence and the second nucleotide sequence encode a chimeric polypeptide sequence which is at least 75% identical, at least 80% identical, at least 85% identical, at least 90% identical, at least 95% identical, or at least 99% identical to SEQ ID NO:26, SEQ ID NO:27, SEQ ID NO:28, SEQ ID NO:29, SEQ ID NO:30, SEQ ID NO:31, SEQ ID NO:32, or SEQ ID NO:33. In some embodiments, the first nucleotide sequence and the second nucleotide sequence encode a chimeric polypeptide sequence according to SEQ ID NO:26, SEQ ID NO:27, SEQ ID NO:28, SEQ ID NO:29, SEQ ID NO:30, SEQ ID NO:31, SEQ ID NO:32, or SEQ ID NO:33.

In some embodiments, a nucleotide sequence encoding a chimeric polypeptide comprising a first nucleotide sequence and a second nucleotide sequence can further contain a third nucleotide sequence encoding a reporter protein or fragment thereof (e.g., a fluorescent protein such as mCherry). Thus, in some embodiments, a first nucleotide sequence can encode a light-induced oligomerization domain comprising NcVVDY50W, a second nucleotide sequence can encode a low complexity domain from a neurodegenerative disease target protein comprising full-length or truncated TDP-43, and a third nucleotide sequence can encode a mCherry protein, wherein the first, second, and third nucleotide sequences encode a chimeric polypeptide sequence which is at least 75% identical, at least 80% identical, at least 85% identical, at least 90% identical, at least 95% identical, or at least 99% identical to SEQ ID NO:34, SEQ ID NO:35, SEQ ID NO:36, SEQ ID NO:37, SEQ ID NO:38, SEQ ID NO:39, SEQ ID NO:40, or SEQ ID NO:41. In some embodiments, the first, second, and third nucleotide sequences encode a chimeric polypeptide sequence according to SEQ ID NO:34, SEQ ID NO:35, SEQ ID NO:36, SEQ ID NO:37, SEQ ID NO:38, SEQ ID NO:39, SEQ ID NO:40, or SEQ ID NO:41. In some embodiments, SEQ ID NO:34 is selected. In some embodiments, SEQ ID NO:41 is selected.

In some embodiments, the first nucleotide sequence can encode a light-induced oligomerization domain comprising a PHR domain of the Arabidopsis Cryptochrome 2 protein (e.g., CRY2OLIG), and the second nucleotide sequence can encode a low complexity domain from a neurodegenerative disease target protein comprising α-synuclein. Thus, in some embodiments, the first nucleotide sequence can encode a light-induced oligomerization domain comprising CRY2OLIG, and the second nucleotide sequence can encode a low complexity domain from a neurodegenerative disease target protein comprising α-synuclein, wherein the first nucleotide sequence and the second nucleotide sequence encode a chimeric polypeptide sequence which is at least 75% identical, at least 80% identical, at least 85% identical, at least 90% identical, at least 95% identical, or at least 99% identical to SEQ ID NO:42 or SEQ ID NO:43. In some embodiments, the first nucleotide sequence and the second nucleotide sequence encode a chimeric polypeptide sequence according to SEQ ID NO:42 or SEQ ID NO:43.

In some embodiments, a nucleotide sequence encoding a chimeric polypeptide comprising a first nucleotide sequence and a second nucleotide sequence can further contain a third nucleotide sequence encoding a reporter protein or fragment thereof (e.g., a fluorescent protein such as mCherry). Thus, in some embodiments, a first nucleotide sequence can encode a light-induced oligomerization domain comprising CRY2OLIG, a second nucleotide sequence can encode a low complexity domain from a neurodegenerative disease target protein comprising α-synuclein, and a third nucleotide sequence can encode a mCherry protein, wherein the first, second, and third nucleotide sequences encode a chimeric polypeptide sequence which is at least 75% identical, at least 80% identical, at least 85% identical, at least 90% identical, at least 95% identical, or at least 99% identical to SEQ ID NO:44, SEQ ID NO:45, or SEQ ID NO:46. In some embodiments, the first, second, and third nucleotide sequences encode a chimeric polypeptide sequence according to SEQ ID NO:44, SEQ ID NO:45, or SEQ ID NO:46. In some embodiments, SEQ ID NO:44 is selected.

In some embodiments, the first nucleotide sequence can encode a light-induced oligomerization domain comprising a LOV photoreceptor domain (e.g., NcVVD, NcVVDY50W, NcLOV, VfAU1, YtvA, EL222, RsLOV, and AsLOV2), and the second nucleotide sequence can encode a low complexity domain from a neurodegenerative disease target protein comprising α-synuclein. Thus, in some embodiments, the first nucleotide sequence can encode a light-induced oligomerization domain comprising NcVVDY50W, and the second nucleotide sequence can encode a low complexity domain from a neurodegenerative disease target protein comprising α-synuclein, wherein the first nucleotide sequence and the second nucleotide sequence encode a chimeric polypeptide sequence which is at least 75% identical, at least 80% identical, at least 85% identical, at least 90% identical, at least 95% identical, or at least 99% identical to SEQ ID NO:47 or SEQ ID NO:48. In some embodiments, the first nucleotide sequence and the second nucleotide sequence encode a chimeric polypeptide sequence according to SEQ ID NO:47 or SEQ ID NO:48.

In some embodiments, a nucleotide sequence encoding a chimeric polypeptide comprising a first nucleotide sequence and a second nucleotide sequence can further contain a third nucleotide sequence encoding a reporter protein or fragment thereof (e.g., a fluorescent protein such as mCherry). Thus, in some embodiments, a first nucleotide sequence can encode a light-induced oligomerization domain comprising NcVVDY50W, a second nucleotide sequence can encode a low complexity domain from a neurodegenerative disease target protein comprising α-synuclein, and a third nucleotide sequence can encode a mCherry protein, wherein the first, second, and third nucleotide sequences encode a chimeric polypeptide sequence which is at least 75% identical, at least 80% identical, at least 85% identical, at least 90% identical, at least 95% identical, or at least 99% identical to SEQ ID NO:49, SEQ ID NO:50, or SEQ ID NO:51. In some embodiments, the first, second, and third nucleotide sequences encode a chimeric polypeptide sequence according to SEQ ID NO:49, SEQ ID NO:50, or SEQ ID NO:51. In some embodiments, SEQ ID NO:51 is selected.

In some embodiments, the first nucleotide sequence is positioned upstream of the second nucleotide sequence. In some embodiments, the first nucleotide sequence is positioned downstream of the second nucleotide sequence.

In some embodiments, where the sequences disclosed herein contain a methionine at the start of the protein, the protein without the methionine is also disclosed. In some embodiments, where the sequences disclosed herein do not contain a methionine at the start of the protein, the protein with the methionine at the start of the protein is also disclosed.

In some embodiments, the nucleotide sequence encoding a chimeric polypeptide comprises a sequence selected from SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, or SEQ ID NO:91.

In some embodiments, disclosed herein is a nucleotide sequence encoding a chimeric polypeptide, comprising: a first nucleotide sequence encoding a light-induced oligomerization domain and a second nucleotide sequence encoding a low complexity domain from a neurodegenerative disease target protein, wherein the low complexity domain from a neurodegenerative disease target protein is a Tau.

In some aspects, the first nucleotide sequence encodes an amino acid sequence that is at least 85% identical to SEQ ID NO:5. In other aspects, the first nucleotide sequence encodes an amino acid sequence that is at least 90% identical to SEQ ID NO:5. In still other aspects, the first nucleotide sequence encodes an amino acid sequence that is at least 95% identical to SEQ ID NO:5. In one embodiment, the first nucleotide sequence encodes an amino acid sequence comprising SEQ ID NO:103.

In one aspect, disclosed herein is a nucleotide sequence encoding a chimeric polypeptide, comprising: a first nucleotide sequence encoding a VVD light-induced oligomerization domain and a second nucleotide sequence encoding an aggregating neurodegenerative disease target polypeptide, wherein the VVD light-induced oligomerization domain comprises mutations I74V and I85V, or mutations corresponding thereto. In some embodiments, the VVD mutations I74V and I85V allow for improved light induced oligomerization, or aggregation, as a result of enhanced dimerization efficiency and/or faster switch-off kinetics. These mutations reduce the limitations of the wild-type VVD system, which include slow on/off kinetics, weak dimerization efficiency, and dark-state activity.

In some aspects, the VVD light-induced oligomerization domain has an N-terminal truncation as compared to a wild-type VVD light-induced oligomerization domain, which truncation results in a more stable VVD polypeptide. Wild-type VVD is provided in SEQ ID NO:105. In some aspects, the N-terminal truncation of VVD is between about 32 residues and about 40 residues. In some aspects, the N-terminal truncation is between about 34 residues and about 38 residues. In some aspects, the N-terminal truncation is about 36-residues.

In some embodiments, the nucleotide sequence encoding the chimeric polypeptide further comprises a sequence encoding a visualization probe binding sequence. In some embodiments, the visualization probe is a fluorescent dye, and in some further embodiments, the visualization probe binding sequence is a modified haloalkane dehalogenase designed to covalently bind to the fluorescent dye. In some aspects, the nucleotide sequence further comprises an inducible expression cassette to allow for inducible expression of the chimeric polypeptide.

Also included are expression vectors that include the nucleotide sequence encoding the chimeric polypeptide as described herein. In some embodiments, the expression vector encoding a chimeric polypeptide is comprised in a plasmid or in a virus or viral vector. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector encoding a chimeric polypeptide is a naked DNA or is comprised in a nanoparticle (e.g., liposomal vesicle, porous silicon nanoparticle, gold-DNA conjugate particle, polyethylenimine polymer particle, cationic peptides, etc.). The expression vectors disclosed herein are, in some embodiments, capable of inducing a neurodegenerative disease pathology in a cell (e.g., inducing aggregation of an aggregating neurodegenerative disease target protein).

Also included herein are polypeptide sequences encoded by the polynucleotide sequences disclosed herein. In one aspect, disclosed herein is a chimeric polypeptide, comprising: a VVD light-induced oligomerization domain and an aggregating neurodegenerative disease target polypeptide, wherein the VVD light-induced oligomerization domain comprises mutations I74V and I85V, or mutations corresponding thereto. In some embodiments, the chimeric polypeptide further comprises a visualization probe binding sequence. In some embodiments, the visualization probe is a fluorescent dye, and in some further embodiments, the visualization probe binding sequence is a modified haloalkane dehalogenase designed to covalently bind to the fluorescent dye. In some embodiments, the VVD light-induced oligomerization domain is connected to the C-terminal end of the aggregating neurodegenerative disease target polypeptide.

In one aspect, disclosed herein is a cell comprising a chimeric polynucleotide, comprising: a first nucleotide sequence encoding a VVD light-induced oligomerization domain and a second nucleotide sequence encoding an aggregating neurodegenerative disease target polypeptide, wherein the VVD light-induced oligomerization domain comprises mutations I74V and I85V, or mutations corresponding thereto. This cell is also referred to herein as a “host cell.” In some embodiments, the cell is a cell which can be affected by a neurodegenerative disease. For example, the cell can be a glial cell or a neuronal cell. In one embodiment, the cell is a mammalian cell. In one embodiment, the cell is a human cell. In one embodiment, the cell is selected from the group consisting of yeast, insect, avian, fish, worm, amphibian, xenopus, bacteria, algae and mammalian cells.

As discussed herein, the chimeric polynucleotides encode, and the chimeric polypeptides include, a VVD light-induced oligomerization domain. Vivid (VVD) is a protein generated in the Neurospora crassa organism and is a photoreceptor that homodimerizes in response to blue light (within the 405-499 nm range). The Light-Oxygen-Voltage-Sensing (LOV) domain of VVD is common throughout many organisms but this domain is very small in this organism. In some aspects, the VVD light-induced oligomerization domain is a Neurospora crassa VVD domain. In one embodiment, the VVD light-induced oligomerization domain comprises SEQ ID NO:103. SEQ ID NO:103 includes mutations I74V and I85V, which are shown in bold. SEQ ID NO:103 is an N-terminal truncated version of Neurospora crassa VVD (residues 37-186). In some embodiments, the VVD polypeptide comprises mutations that correspond to the I74V and I85V mutations and is otherwise at least 75% identical, at least 80% identical, at least 85% identical, at least 90% identical, at least 95% identical, or at least 99% identical to SEQ ID NO:103. “Corresponding mutations” have a same or similar function as the first named mutations and allow for improved light induced oligomerization.

The neurodegenerative disease target polypeptide can be any polypeptide selected from the group consisting of TDP-43, Alpha synuclein, Tau, Fus, TIA1, SOD1, Huntingtin, Ataxin 2, hnRNPA1, and hnRNPA2B1. In some aspects, the aggregating neurodegenerative disease target polypeptide is a tau polypeptide. In some embodiments, the tau polypeptide comprises SEQ ID NO:104. In some embodiments, the tau polypeptide is at least 75% identical, at least 80% identical, at least 85% identical, at least 90% identical, at least 95% identical, or at least 99% identical to SEQ ID NO:104. In some embodiments, the tau polypeptide comprises one or more mutations. For example, the tau polypeptide can include one or more mutations within a hexapeptide region, such as amino acids 275-280 or 306-311.In some embodiments, the tau polypeptide comprises a pro-aggregation mutation (\hat{ }K280). In other embodiments, the tau polypeptide comprises one or more anti-175 aggregation mutations (\hat{ }K280, I277P, I308P). Furthermore, included here are all isoforms of tau polypeptides. Tau is expressed in six different isoforms in the adult human brain. Tau isoforms differ in the number of amino (N)-terminal inserts (0N, 1N, 2N) and contain either three or four 31-32-amino acid repeat sequences (R1-R4). NFTs in progressive supranuclear palsy predominantly contain 4R-tau and Pick disease inclusions contain mostly 3R-tau, whereas NFTs in AD contain a mixture of 4R-tau and 3R-tau. Accordingly, included herein are tau polypeptides having any combination of amino-terminal inserts and amino acid repeat sequences.

### Methods

In one aspect, disclosed herein is a method of inducing a neurodegenerative disease pathology in a cell, comprising the steps:
a. introducing into the cell an expression vector encoding a chimeric polypeptide, comprising:
b. a first nucleotide sequence encoding a light-induced oligomerization domain and a second nucleotide sequence encoding a low complexity domain from a neurodegenerative disease target protein, wherein the first nucleotide sequence is operably linked to a promoter;
c. expressing the chimeric polypeptide; and
d. inducing oligomerization of the chimeric polypeptide by stimulation with blue light.

In another aspect, disclosed herein is a method of screening for an agent that modulates protein aggregation, comprising the steps:
a. introducing into a cell an expression vector encoding a chimeric polypeptide, comprising:
b. a first nucleotide sequence encoding a light-induced oligomerization domain and a second nucleotide sequence encoding a low complexity domain from a neurodegenerative disease target protein, wherein the first nucleotide sequence is operably linked to a promoter;
c. expressing the chimeric polypeptide;
d. introducing the agent into a culture media comprising the cell;
e. inducing oligomerization of the chimeric polypeptide by stimulation with blue light; and
f. determining modulation of protein aggregation by the agent.

In one embodiment, the light-induced oligomerization domain is selected from the group consisting of a CRY2 PHR domain (for example, CRY2PHR, CRY2OLIG) or a light-oxygen-voltage-sensing (LOV) domain (for example, NcVVD, NcVVDY50W, VfAU1, YtvA, EL222, RsLOV, AsLOV2). “NcVVD” is also referred to herein as “VVD light-induced oligomerization domain.”

In one embodiment, the light-induced oligomerization domain is selected from the group consisting of CRYPHR, CRY2OLIG, NcVVD, NcVVDY50W, and NcLOV. In one embodiment, the light-induced oligomerization domain is selected from the group consisting of CRYPHR, CRY2OLIG, NcVVD, NcVVDY50W, NcLOV, VfAU1, YtvA, EL222, RsLOV, and AsLOV2. In one embodiment, the light-induced oligomerization domain is selected from the list of domains in Table 2. In one embodiment, the light-induced oligomerization domain is selected from a variant of CRYPHR, CRY2OLIG, NcVVD, NcVVDY50W, NcLOV, VfAU1, YtvA, EL222, RsLOV, or AsLOV2. In one embodiment, the light-induced oligomerization domain is selected from a fragment of CRYPHR, CRY2OLIG, NcVVD, NcVVDY50W, NcLOV, VfAU1, YtvA, EL222, RsLOV, or AsLOV2.

In one embodiment, the light-induced oligomerization domain is NcVVDY50W. In one embodiment, the light-induced oligomerization domain is CRY2OLIG. In one embodiment, the light-induced oligomerization domain is CRYPHR. In one embodiment, the light-induced oligomerization domain comprises a LOV domain from the VVD protein. In one embodiment, the light-induced oligomerization domain comprises a LOV domain from the LOV protein. In one embodiment, the light-induced oligomerization domain comprises a PHR domain. In one embodiment, the light-induced oligomerization domain comprises a PHR domain, from the CRY2 protein. In one embodiment, the light-induced oligomerization domain is VfAU1. In one embodiment, the light-induced oligomerization domain is YtvA. In one embodiment, the light-induced oligomerization domain is EL222. In one embodiment, the light-induced oligomerization domain is RsLOV. In one embodiment, the light-induced oligomerization domain is AsLOV2.

In one embodiment, the low complexity domain from a neurodegenerative disease target protein is selected from the group consisting of TDP-43, Alpha synuclein, Tau, Fus, TIA1, SOD1, Huntingtin, Ataxin 2, hnRNPA1, and hnRNPA2B1. In one embodiment, the low complexity domain from a neurodegenerative disease target protein is selected from the group consisting of TDP-43, Alpha synuclein, Tau, Fus, TIA1, SOD1, Huntingtin, Ataxin 2, hnRNPA1, hnRNPA2B1, EWS RNA Binding Protein 1, and TATA box binding protein factor 15. In one embodiment, the low complexity domain from a neurodegenerative disease target protein is selected from a variant of TDP-43, Alpha synuclein, Tau, TIA1, SOD1, Huntingtin, Ataxin 2, hnRNPA1, hnRNPA2B1, EWS RNA Binding Protein 1, or TATA box binding protein factor 15. In one embodiment, the low complexity domain from a neurodegenerative disease target protein is selected from a fragment of TDP-43, Alpha synuclein, Tau, TIA1, SOD1, Huntingtin, Ataxin 2, hnRNPA1, hnRNPA2B1, EWS RNA Binding Protein 1, or TATA box binding protein factor 15. In one embodiment, the fragment of TDP-43, Alpha synuclein, Tau, TIA1, SOD1, Huntingtin, Ataxin 2, hnRNPA1, hnRNPA2B1, EWS RNA Binding Protein 1, or TATA box binding protein factor 15 comprises the low complexity domain within each neurodegenerative disease target protein.

In one embodiment, the low complexity domain from a neurodegenerative disease target protein is selected from Table 3. In one embodiment, the low complexity domain is from any neurodegenerative disease target protein that aggregates in neurodegenerative diseases.

In one embodiment, the low complexity domain from a neurodegenerative disease target protein is selected from the group consisting of an amino acid sequence with at least 90% identity to TDP-43, Alpha synuclein, Tau, Fus, TIA1, SOD1, Huntingtin, Ataxin 2, hnRNPA1, hnRNPA2B1, EWS RNA Binding Protein 1, and TATA box binding protein factor 15. In one embodiment, the low complexity domain from a neurodegenerative disease target protein is selected from the group consisting of an amino acid sequence with at least 90% identity to a low complexity domain from a neurodegenerative disease target protein is selected from Table 3. In one embodiment, the low complexity domain from a neurodegenerative disease target protein is selected from an orthologue of the group consisting of TDP-43, Alpha synuclein, Tau, Fus, TIA1, SOD1, Huntingtin, Ataxin 2, hnRNPA1, hnRNPA2B1, EWS RNA Binding Protein 1, and TATA box binding protein factor 15.

In one embodiment, the low complexity domain from a neurodegenerative disease target protein is selected from the group consisting of an amino acid sequence with at least 60% (for example, at least 60%, at least 70%, at least 80%, at least 90%, at least 95%) identity to TDP-43, Alpha synuclein, Tau, Fus, TIA1, SOD1, Huntingtin, Ataxin 2, hnRNPA1, hnRNPA2B1, EWS RNA Binding Protein 1, and TATA box binding protein factor 15 (or a fragment thereof).

In one embodiment, the low complexity domain from a neurodegenerative disease target protein is TDP-43. In one embodiment, the low complexity domain from a neurodegenerative disease target protein is Alpha synuclein. In one embodiment, the low complexity domain from a neurodegenerative disease target protein is Tau. In one embodiment, the low complexity domain from a neurodegenerative disease target protein is Fus. In one embodiment, the low complexity domain from a neurodegenerative disease target protein is TIA1. In one embodiment, the low complexity domain of a neurodegenerative disease target protein is SOD1. In one embodiment, the low complexity domain from a neurodegenerative disease target protein is Huntingtin. In one embodiment, the low complexity domain from a neurodegenerative disease target protein is Ataxin 2. In one embodiment, the low complexity domain from a neurodegenerative disease target protein is hnRNPA1. In one embodiment, the low complexity domain from a neurodegenerative disease target protein is hnRNPA2B1. In one embodiment, the low complexity domain from a neurodegenerative disease target protein is EWS RNA Binding Protein 1. In one embodiment, the low complexity domain from a neurodegenerative disease target protein is TATA box binding protein factor 15.

In one embodiment, the cell is a mammalian cell. In one embodiment, the cell is a human cell. In one embodiment, the cell is selected from the group consisting of yeast, insect, avian, fish, worm, amphibian, xenopus, bacteria, algae and mammalian cells. In one embodiment, disclosed herein is a non-human transgenic organism, wherein the organism is an insect, fish, bird, worm, amphibian, xenopus, or non-human mammal.

To induce a neurodegenerative disease pathology refers to the action of bringing about the neurodegenerative disease pathology, or increasing the phenotype, symptoms, or severity of the neurodegenerative disease pathology, as compared to refraining from performing the selected action.

The neurodegenerative disease pathology can include an array of pathologies known to be or suspected to be associated with any one or more neurodegenerative diseases. Examples of such pathologies include, but are not limited to, protein aggregation in the cytoplasm of a cell, mislocalization of nuclear proteins to, for example, the cytoplasm, increased expression of ubiquitin, cell degeneration and/or death, extracellular Amyloid Beta (AB) aggregation, and/or intracellular and/or cytoplasmic aggregation of Tau protein. Protein aggregates can, in some embodiments, be colocalized with p62 protein, can be hyper-phosphorylated, can include endogenous protein comprising the low complexity domain from a neurodegenerative disease target protein (e.g., endogenous TDP-43), or combinations thereof.

Increased ubiquitination or increased expression of ubiquitin can be a phenotypic feature of neurodegenerative diseases. Ubiquitin has numerous cellular roles, including “tagging” proteins (e.g., by covalent linkage) for degradation in a proteasome. Increased ubiquitin expression in a cell is typically compared to a control. In some embodiments, a cell having increased ubiquitin has at least 50% increased ubiquitin expression compared to a control. In some embodiments, a cell having increased ubiquitin has at least 60%, at least 70%, at least 80%, at least 90%, at least 95%, at least 98%, or at least 99% increased ubiquitin expression compared to a control.

Ubiquitin expression in a cell can be determined at the transcriptional level, the translational level, or combinations thereof, and can be measured via a wide array of methods used to measure gene or polypeptide expression levels. In some embodiments, ubiquitin expression can be measured at the gene transcription level. For example and without limitation, levels of ubiquitin mRNA transcripts can be determined by radiation absorbance (e.g., ultraviolet light absorption at 260, 280, or 230 nm), quantification of fluorescent dye or tag emission (e.g., ethidium bromide intercalation), quantitative polymerase chain reaction (qPCR) of cDNA produced from mRNA transcripts, southern blot analysis, gene expression microarray, or other suitable methods. Increased levels of mRNA transcripts can be used to infer or estimate increased levels of polypeptide expression. In some embodiments, ubiquitin expression can be measured at the post-translational level. For example and without limitation, levels of ubiquitin polypeptide can be determined by radiation absorbance (e.g., ultraviolet light), bicinchoninic acid (BCA) assay, Bradford assay, biuret test, Lowry method, Coomassie-blue staining, functional or enzymatic assay, immunodetection methods and/or Western blot analysis, or other suitable methods.

As used herein, the term “introducing,” “introduce,” and grammatical variations thereof, as it relates to introducing an expression vector into a cell, refers to any method suitable for transferring the expression vector into the cell. The term includes as examples, but is not limited to, conjugation, transformation/transfection (e.g., divalent cation exposure, heat shock, electroporation), nuclear microinjection, incubation with calcium phosphate polynucleotide precipitate, high velocity bombardment with polynucleotide-coated microprojectiles (e.g., via gene gun), lipofection, cationic polymer complexation (e.g., DEAE-dextran, polyethylenimine), dendrimer complexation, mechanical deformation of cell membranes (e.g., cell-squeezing), sonoporation, optical transfection, impalefection, hydrodynamic polynucleotide delivery, Agrobacterium-mediated transformation, transduction (e.g., transduction with a virus or viral vector), natural or artificial competence, protoplast fusion, magnetofection, nucleofection, or combinations thereof. An introduced expression vector, or a polynucleotide therefrom, can be genetically integrated or exist extrachromosomally.

A range of blue light wavelengths can be used in the disclosed methods. In one embodiment, the blue light has a wavelength from about 400 nm to about 500 nm. In one embodiment, the blue light has a wavelength from about 405 nm to about 499 nm. In one embodiment, the blue light has a wavelength from about 420 nm to about 490 nm. In one embodiment, the blue light has a wavelength from about 450 nm to about 490 nm. In one embodiment, the blue light has a wavelength from about 460 nm to about 495 nm. In one embodiment, the blue light has a wavelength of about 488 nm. In one embodiment, the blue light has a wavelength of about 475 nm. In one embodiment, the blue light has a wavelength of about 465 nm.

In one embodiment, the blue light has a wavelength of about 405 nm, about 410 nm, about 415 nm, about 420 nm, about 425 nm, about 430 nm, about 435 nm, about 440 nm, about 445 nm, about 450 nm, about 455 nm, about 460 nm, about 465 nm, about 470 nm, about 475 nm, about 480 nm, about 485 nm, about 490 nm, about 495 nm, or about 500 nm.

The methods can include various degrees of blue light stimulation. In some embodiments, the stimulation is acute or, optionally, chronic. Acute stimulation refers stimulation with pulses of blue light from about 0.2 to about 60 seconds, wherein the wavelength of the blue light can be any herein disclosed blue light wavelength. In some embodiments, the acute stimulation includes pulses of blue light from about 0.5 second to about 30 seconds, from about 1 second to about 20 seconds, or about 5 seconds. The blue light can be provided by a blue light source or a broad-spectrum light source filtered for the disclosed wavelengths.

In some embodiments, acute stimulation can result in temporary aggregation of a light-induced oligomerization domain (e.g., cytoplasmic prion-like domains/LCD/IDD protein fragments). Temporary aggregation, in some embodiments, includes protein aggregation observable by the herein disclosed methods for less than about twenty minutes or, optionally, less than about fifteen minutes, less than about ten minutes, or about five minutes or less. In some embodiments, acute stimulation does not result in aggregation of cytoplasmic prion-like domains/LCD/IDD protein fragments for twenty minutes or more.

In some embodiments, acute stimulation can result in aggregation of a light-induced oligomerization domain which is shorter in duration than aggregation of a light-induced oligomerization domain fused with a low complexity domain from a neurodegenerative disease target protein. In some embodiments, acute stimulation can result in aggregation of a light-induced oligomerization domain fused with a low complexity domain from a neurodegenerative disease target protein which is shorter in duration than aggregation of the same protein having an amino acid mutation known to cause or be associated with a neurodegenerative disease (e.g., TDP-43 Q331K).

Chronic stimulation is defined by exposure to blue light having a wavelength from about 400 nm to about 500 nm for a duration of about 1 minute or longer (for example, at least 1 minute, at least 5 minutes, at least 10 minutes, at least 30 minutes, at least 60 minutes, at least 2 hours, at least 4 hours, at least 8 hours, at least 12 hours, at least 24 hours, at least 36 hours, or more) from about 0.1 mW/cm² to 8 mW/cm² (within 400 nm-500 nm wavelength).

The methods disclosed herein can, in some embodiments, induce a neurodegenerative disease pathology in a cell (e.g., aggregation of a protein) without substantially altering the expression level of a protein involved in the neurodegenerative disease pathology. For example and without limitation, the methods can induce aggregation of a chimeric polypeptide comprising TDP-43 (which can include aggregation of endogenous TDP-43) without substantially increasing or decreasing the expression level of endogenous TDP-43. In some or further embodiments, the methods can induce a neurodegenerative disease pathology in a cell using a wild-type form of a low complexity domain from a neurodegenerative disease target protein. Thus, in some embodiments, the low complexity domain from a neurodegenerative disease target protein does not include a mutation which differs from the wild-type sequence and which is known or suspected to cause or be associated with inducing a neurodegenerative disease pathology in a cell. For example and without limitation, the methods can induce aggregation of a chimeric polypeptide comprising wild-type TDP-43 protein, or fragment thereof, which does not contain a mutation such as Q331K, known to cause or be associated with ALS.

In one aspect, disclosed herein is a method of inducing aggregation of a neurodegenerative disease target polypeptide in a cell, comprising the steps:
a. introducing into the cell an expression vector encoding a chimeric polypeptide, comprising:
b. a first nucleotide sequence encoding a VVD light-induced oligomerization domain and a second nucleotide sequence encoding an aggregating neurodegenerative disease target polypeptide, wherein the VVD light-induced oligomerization domain comprises mutations I74V and I85V, or mutations corresponding thereto;
c. expressing the chimeric polypeptide; and
d. inducing aggregation of the chimeric polypeptide by stimulation with blue light.

In one aspect, disclosed herein is a method of inducing a neurodegenerative disease pathology in a cell, comprising the steps:
a. introducing into the cell an expression vector encoding a chimeric polypeptide, comprising:
b. a first nucleotide sequence encoding a light-induced oligomerization domain and a second nucleotide sequence encoding a low complexity domain from a neurodegenerative disease target protein, wherein the first nucleotide sequence is operably linked to a promoter and wherein the low complexity domain from a neurodegenerative disease target protein is a Tau;
c. expressing the chimeric polypeptide; and
d. inducing oligomerization of the chimeric polypeptide by stimulation with blue light.

In certain embodiments, the first nucleotide sequence encodes an amino acid sequence that is at least 85% identical to SEQ ID NO:5. In other embodiments, the first nucleotide sequence encodes an amino acid sequence that is at least 90% identical to SEQ ID NO:5. In still other embodiments, the first nucleotide sequence encodes an amino acid sequence that is at least 95% identical to SEQ ID NO:5. In one embodiment, the first nucleotide sequence encodes an amino acid sequence comprising SEQ ID NO:103. In some embodiments, the Tau comprises SEQ ID NO:104.

Included herein is a method of screening for an agent that modulates protein aggregation, comprising the steps:
a. introducing into a cell an expression vector encoding a chimeric polypeptide, comprising a first nucleotide sequence encoding a light-induced oligomerization domain and a second nucleotide sequence encoding a low complexity domain from a neurodegenerative disease target protein, wherein the first nucleotide sequence is operably linked to a promoter and wherein the low complexity domain from a neurodegenerative disease target protein is a Tau;
b. expressing the chimeric polypeptide;
c. introducing the agent into a culture media comprising the cell;
d. inducing oligomerization of the chimeric polypeptide by stimulation with blue light; and
e. determining modulation of protein aggregation by the agent.

In certain embodiments, the first nucleotide sequence encodes an amino acid sequence that is at least 85% identical to SEQ ID NO:5. In other embodiments, the first nucleotide sequence encodes an amino acid sequence that is at least 90% identical to SEQ ID NO:5. In still other embodiments, the first nucleotide sequence encodes an amino acid sequence that is at least 95% identical to SEQ ID NO:5. In one embodiment, the first nucleotide sequence encodes an amino acid sequence comprising SEQ ID NO:103. In some embodiments, the Tau comprises SEQ ID NO:104.

In one aspect, disclosed herein is a method of screening for an agent that modulates solubility or aggregation of a neurodegenerative disease target polypeptide, comprising the steps:
a. introducing into a cell an expression vector encoding a chimeric polypeptide, comprising:
b. a first nucleotide sequence encoding a VVD light-induced oligomerization domain and a second nucleotide sequence encoding an aggregating neurodegenerative disease target polypeptide, wherein the VVD light-induced oligomerization domain comprises mutations I74V and I85V, or mutations corresponding thereto;
c. expressing the chimeric polypeptide;
d. introducing the agent into a culture media comprising the cell;
e. inducing aggregation of the chimeric polypeptide by stimulation with blue light; and
f. determining modulation of solubility or aggregation by the agent.

In some embodiments, the method screens for an agent that modulates aggregation of the neurodegenerative disease target polypeptide. In some embodiments, the method screens for an agent that modulates solubility of the neurodegenerative disease target polypeptide. In each of the methods disclosed herein, in some embodiments, the nucleotide sequence encoding the chimeric polypeptide further comprises a sequence encoding a visualization probe binding sequence. In some embodiments, the visualization probe is a fluorescent dye, and in some further embodiments, the visualization probe binding sequence is a modified haloalkane dehalogenase designed to covalently bind to the fluorescent dye.

In some aspects of the methods, the VVD light-induced oligomerization domain is a Neurospora crassa VVD domain. In one embodiment, the VVD light-induced oligomerization domain comprises SEQ ID NO:103. SEQ ID NO:103 includes mutations I74V and I85V, which are shown in bold. SEQ ID NO:103 is an N-terminal truncated version of Neurospora crassa VVD (residues 37-186). In some embodiments, the VVD polypeptide comprises mutations that correspond to the I74V and I85V mutations and is otherwise at least 75% identical, at least 80% identical, at least 85% identical, at least 90% identical, at least 95% identical, or at least 99% identical to SEQ ID NO:103. “Corresponding mutations” have the same function and allow for improved light induced oligomerization as a result of decreased host cell heating.

The neurodegenerative disease target polypeptide used in the methods can be any polypeptide selected from the group consisting of TDP-43, Alpha synuclein, Tau, Fus, TIA1, SOD1, Huntingtin, Ataxin 2, hnRNPA1, and hnRNPA2B1. In some aspects, the aggregating neurodegenerative disease target polypeptide is a tau polypeptide. In some embodiments, the tau polypeptide comprises SEQ ID NO:104. In some embodiments, the tau polypeptide is at least 75% identical, at least 80% identical, at least 85% identical, at least 90% identical, at least 95% identical, or at least 99% identical to SEQ ID NO:104.

A range of blue light wavelengths can be used in the disclosed methods. In one embodiment, the blue light has a wavelength from about 400 nm to about 500 nm. In one embodiment, the blue light has a wavelength from about 405 nm to about 499 nm. In one embodiment, the blue light has a wavelength from about 420 nm to about 490 nm. In one embodiment, the blue light has a wavelength from about 450 nm to about 490 nm. In one embodiment, the blue light has a wavelength from about 460 nm to about 495 nm. In one embodiment, the blue light has a wavelength of about 488 nm. In one embodiment, the blue light has a wavelength of about 475 nm. In one embodiment, the blue light has a wavelength of about 465 nm.

In one embodiment, the blue light has a wavelength of about 405 nm, about 410 nm, about 415 nm, about 420 nm, about 425 nm, about 430 nm, about 435 nm, about 440 nm, about 445 nm, about 450 nm, about 455 nm, about 460 nm, about 465 nm, about 470 nm, about 475 nm, about 480 nm, about 485 nm, about 490 nm, about 495 nm, or about 500 nm.

The methods can include various degrees of blue light stimulation. In some embodiments, the stimulation is from about 15 minutes to about 48 hours. In some embodiments, the stimulation is about 1 hour, about 2 hours, about 3 hours, about 4 hours, about 5 hours, about 6 hours, about 7 hours, about 8 hours, about 9 hours, about 10 hours, about 11 about hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In some embodiments, the stimulation is about 20 hours to about 28 hours. In some embodiments, the stimulation is about 22 hours to about 26 hours. In some embodiments, the stimulation is about 24 hours. In some embodiments, the stimulation is about 24 to about 30 hours. The blue light can be provided by a blue light source or a broad-spectrum light source filtered for the disclosed wavelengths.

### EXAMPLES

The following examples are set forth below to illustrate the compounds, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Optogenetic Induction of Neurodegenerative Disease Pathologies

The world is aging. By the year 2050, the proportion of individuals over the age of 60 will have doubled to 2 billion from 605 million in 2000. Unfortunately, aging is the single greatest risk factor for developing a fatal neurodegenerative disease. In turn, the number of individuals with dementias such as Alzheimer's disease (AD), Lewy Body Dementia (LBD), Frontotemporal Dementia (FTD), and movement disorders such as Parkinson's Disease (PD) and Amyotrophic Lateral Sclerosis (ALS) will significantly increase. Nearly 6.5 million individuals within the United States are currently living with one of these diseases and the associated costs are unsustainable. In the United States, the current economic burden of AD, PD, and ALS is an estimated $241 billion dollars per year. AD and ALS/FTD patients can incur personal medical costs upwards of $100,000-$250,000 per year. For AD, it is estimated that 13.8 million individuals in the United States will have been diagnosed by 2050, up from 4.7 million in 2010, while the worldwide number of ALS cases will rise ˜31% by 2040. Moreover, no effective treatment currently exists for these disorders.

Two decades of genetic analysis has uncovered a number of neurodegenerative disease-associated mutations. However, these are found in only a fraction of AD, LBD, FTD, PD, and ALS patients (Table 1).

TABLE 1
List of common neurodegenerative diseases, pathology and contribution of genetic components.

| | | Affected | Life Expectancy | Primary Aggregate | Genetic |
|---|---|---|---|---|---|
| Disease | Symptoms | CNS Regions | (years) | Pathology | Cause |
| AD | Memory loss, confusion | Entorhinal cortex, | 8-10 | Tau (100%), Aβ (100%) | 2-3% |
| | | Hippocampus | | | |
| FTD | Personality change, | Frontal lobe, | 7-10 | TDP-43 (45%), | 20-25% |
| | compulsivity, aphasia | Temporal lobe | | Tau (45%), Fus (5%) | |
| PD | Stiffness, muscle | Substantia | 15-20+ | α-synuclein (99%) | 10% |
| | rigidity, slowing of | nigra | | | |
| | movement | | | | |
| ALS | Muscle weakness, | Motor cortex, | 3-5 | TDP-43 (97%), | 10% |
| | paralysis | Spinal cord | | SOD1(2%), Fus (1%), | |

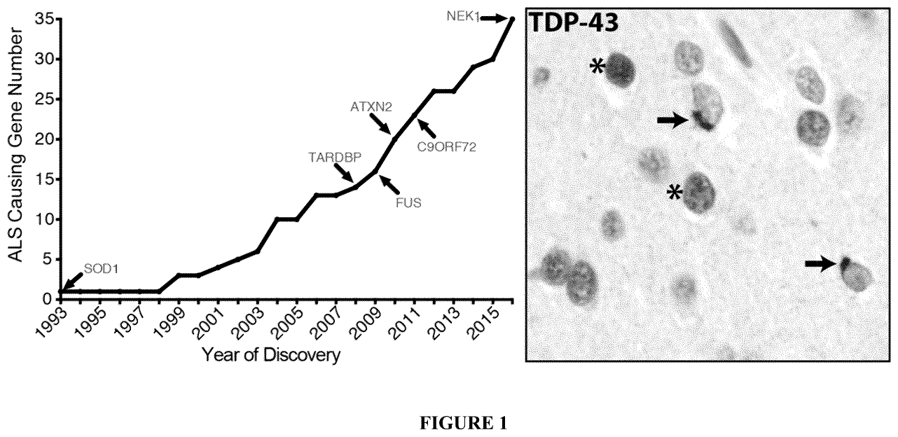

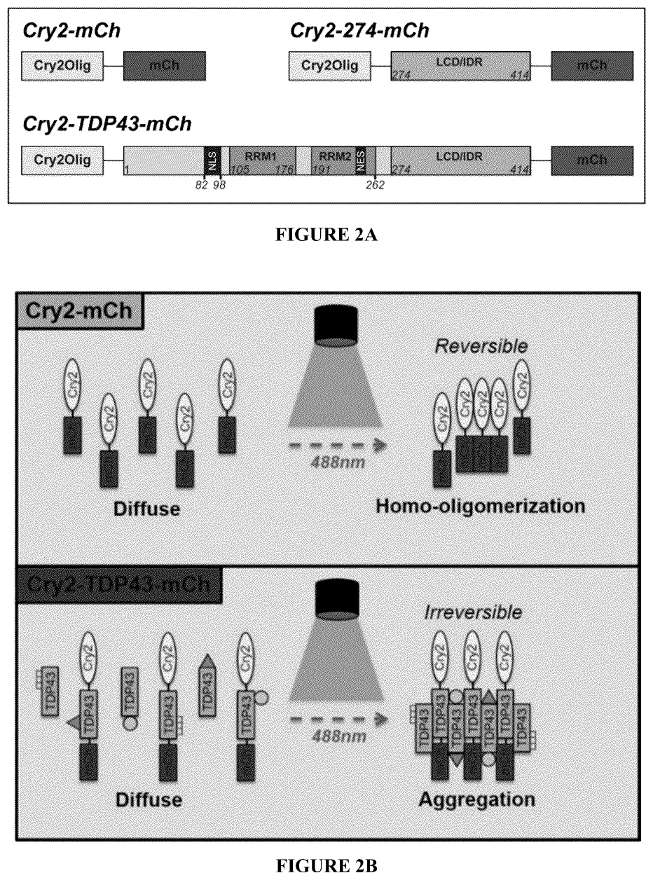

Table 1 describes a selection of neurodegenerative diseases, the primary symptoms, the affected region of the central nervous system (CNS), the aggregate neuropathology observed in patient CNS, percent of patients exhibiting this pathology and the percent of patients that harbor some genetic component of the disease (known or unknown mutation within the family). Alzheimer's disease (AD) is characterized by memory loss and confusion due to degeneration of the hippocampus and entorhinal cortex. Only 1-3% of patients harbor a causal genetic mutation; however, all patients show a common aggregate pathology in the affected tissue. This is characterized by the extracellular Amyloid Beta (AB) aggregates and intracellular and cytoplasmic aggregations of the Tau protein. Frontotemporal Dementia (FTD) is characterized by the progressive degeneration of the frontal and temporal lobes and while 20-25% of patients can harbor a genetic cause of the disease, the 80-75% of patients have no family history. However, all patients exhibit some form of cytoplasmic protein aggregation that are comprised of either TDP-43 (45% of cases), Tau (45% of cases), or FUS (5% of cases) proteins. Parkinson's Disease (PD), is characterized by the progressive degeneration of dopaminergic neurons in the substantia nigra and while only 10% of patients have a known genetic cause, nearly all patients (99%) show cytoplasmic aggregation of the α-synuclein protein. Amyotrophic Lateral Sclerosis (ALS) is characterized by the rapid degeneration of the motor neurons in the motor cortex in the spinal cord leading patient paralysis and death. Only 10% of ALS patients have a family history of the disease while nearly 90% of cases occur sporadically. To date, nearly 35 causative genetic mutations have been identified in various genes (see FIG. 1), however despite this genetic diversity, nearly all ALS patients exhibit the same neuropathology. Nearly 97% of ALS patients show the cytoplasmic mislocalization of the predominantly nuclear TDP-43 protein in the affected regions. Patients with mutations in the SOD1 or FUS gene also show cytoplasmic aggregations of these proteins but this occurs in only 2% and 1% of all ALS cases, respectively.

Together, this table highlights that the principal unifying factor of many neurodegenerative diseases are the neuropathological hallmarks. These neuropathologies primarily occur in the form of intracellular protein aggregates and in the vast majority of cases these intracellular protein aggregations form regardless of any known genetic cause. Moreover, these neuropathologies predominantly form in the region and cell types most affected in each disease.

Interestingly, despite the lack of a common known genetic or environmental cause, these are universal neuropathological features among patients for each disease. Thus, the vast majority of patients (90-100% of all patients depending on the disease) exhibit the same pathology in the central nervous system despite no known genetic cause. These pathological hallmarks manifest in the form of insoluble protein clumps or aggregates in the central nervous system (Table 1, FIG. 1).

To date, it is not possible to accurately model neurodegenerative disease aggregation that mimics human neuropathology. For each neurodegenerative disease, there is a primary component of the intracellular protein aggregate which harbor protein domains which make these proteins aggregation prone when in a high local concentration (also known as: prion-like domain, low complexity domain, intrinsically disordered domain, intrinsically disordered region). Therefore, current methods to model these neurodegenerative disease aggregates rely on overexpressing these aggregate-prone proteins in vitro or in vivo that comprise the neurodegenerative disease aggregates as the high cellular concentration can, in some models, form aggregates. In addition to, or alternatively, another method of modeling these diseases is to express mutated forms of proteins that comprise neurodegenerative disease aggregates. These mutations are found in a very small subset of patients and typically enhance the ability of the protein to aggregate. Unfortunately, none of these methods recapitulate the cellular environment of patients because the vast majority of patients do not harbor any disease-causing mutation, nor do they grossly overexpress the components of the aggregates. In fact, despite gross overexpression of these proteins, many models still do not exhibit the human neuropathology. This disconnect between the patient biology and model systems has significantly contributed to the lack of translatability of current neurodegenerative disease models.

Described in this example are novel methods to spatially and temporally induce protein aggregates without simple overexpressing of aggregate prone genes or expressing mutant forms of these genes. Thus, this method better recapitulates the human disease condition. Here, an innovative approach has been undertaken to address this challenging biological problem by harnessing the power of optogenetics (controlling protein function with light). A series of novel DNA arrangements have been constructed that can induce neurodegenerative disease pathology as observed in patients only when the cells are exposed to specific light stimuli. This method is used to create in vitro and in vivo model systems to mimic the neuropathology observed in patients and induce this pathology only after the cells are exposed to specific light wavelengths. This novel approach can transform neurodegenerative disease modeling and, unlike the expression of mutant transgenes, can be used to generate various disease models that are applicable to the vast majority of patients and are temporally and spatially inducible.

A series of DNA arrangements have been developed comprising the PHR domain (CRY2OLIG or CRY2PHR) or light-oxygen-voltage-sensing (LOV) domain (NcVVD, NcVVDY50W, Vfau1, YtvA, EL222, RsLOV, AsLOV2) which cluster or homodimerize, respectively, in response to blue light exposure (Table 2) and the DNA sequence of genes that encode for proteins that contain low complexity domains (LCDs) and comprise neurodegenerative disease protein aggregates (Table 3).

TABLE 2
List of photoreceptors/light-induced oligomerization domains to optogenetically
induce neurodegenerative pathologies in cells with blue light exposure.

| | Protein | | Light | Light | |
|---|---|---|---|---|---|
| Nomenclature | Domain | Organism | Stimuli (nm) | Response | Features |
| CRYPHR | Photolyase homology | Arabidopsis | 405-499 | Homo- | Endogenous |
| | region (PHR) | | | oligomerization | protein domain |
| CRY2OLIG | Photolyase | Arabidopsis | 405-499 | Homo- | E490G Mutation |
| | homology region | | | oligomerization | to enhance |
| | (PHR) with E490A | | | | clustering |
| | mutation | | | | |
| NcVVD | Light-oxygen- | Neurospora | 405-499 | Homo- | LOV domain of |
| | voltage-sensing | | | dimerization | VVD gene |
| | (LOV) domain | | | | |
| NcVVDY50W | Light-oxygen- | Neurospora | 405-499 | Homo- | VVD Y50G |
| | voltage-sensing | | | dimerization | Mutation to |
| | (LOV) domain | | | | enhance |
| | | | | | clustering |
| NcLOV | Light-oxygen- | Neurospora | 405-499 | Homo- | Clustering LOV |
| | voltage-sensing | | | dimerization | domain from |
| | (LOV) domain | | | | NcVVD with no |
| | | | | | linker |
| Vfau1 | Light-oxygen- | Vucheria | 405-499 | Homo- | |
| | voltage-sensing | frigida | | dimerization | |
| | (LOV) domain | | | | |
| YtvA | Light-oxygen- | Bacillus | 405-499 | Homo- | |
| | voltage-sensing | subtillis | | dimerization | |
| | (LOV) domain | | | | |
| EL222 | Light-oxygen- | Erythrobacter | 405-499 | Homo- | |
| | voltage-sensing | litoralis | | dimerization | |
| | (LOV) domain | | | | |
| RsLOV | Light-oxygen- | Rhodobacter | 405-499 | Homo- | |
| | voltage-sensing | sphaeroides | | dimerization | |
| | (LOV) domain | | | | |
| AsLOV2 | Light-oxygen- | Avena sativa | 405-499 | Intramolecular | |
| | voltage-sensing | | | conformational | |
| | (LOV) domain | | | change | |

Table 2 shows a list of photoreceptors to be employed to optogenetically induce neurodegenerative pathologies in cells upon blue light exposure. Variations of the PHR domain of the Cryptochrome 2 protein found in the Arabidopsis plant will be one family of photoreceptors employed (named CRY2PHR and CRY2OLIG in this document). The PHR domain of the CRY2 protein and its variants have the ability to cluster/homo-oligomerize for ˜5 minutes when exposed to a single pulse of blue light (within the 405-499 nm range). The CRY2PHR is an endogenous protein sequence found in Arabidopsis. While the CRY2OLIG is the endogenous amino acid sequence but has an E490G mutation and has been shown to exhibit a slightly increased efficiency of clustering upon blue light stimulation. The self-binding of CRY2PHR and CRY2OLIG act through the same mechanism and requires intracellular FAD+. Various arrangements of CRY2OLIG and target proteins that comprise neuropathological protein aggregates have been generated to show modulation of the light-induced clustering properties of CRY2OLIG to induce neurodegenerative disease pathology of both predominantly cytoplasmic and predominantly nuclear proteins. This includes inducing the cytoplasmic mislocalization of nuclear proteins as observed in patient neuropathology. In addition, these protein domains have been used to induce the aggregation of intrinsically disordered domains/prion-like domains/low complexity domains of truncated versions of these neurodegenerative proteins.

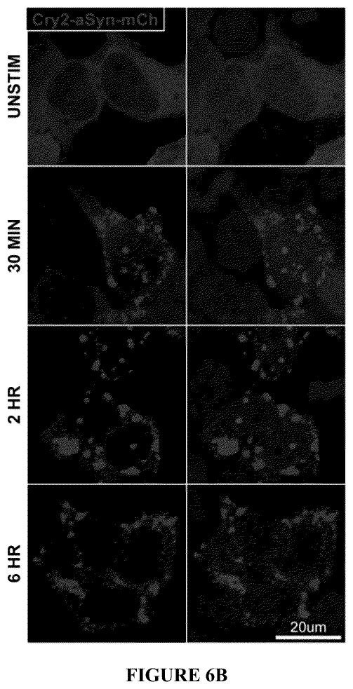

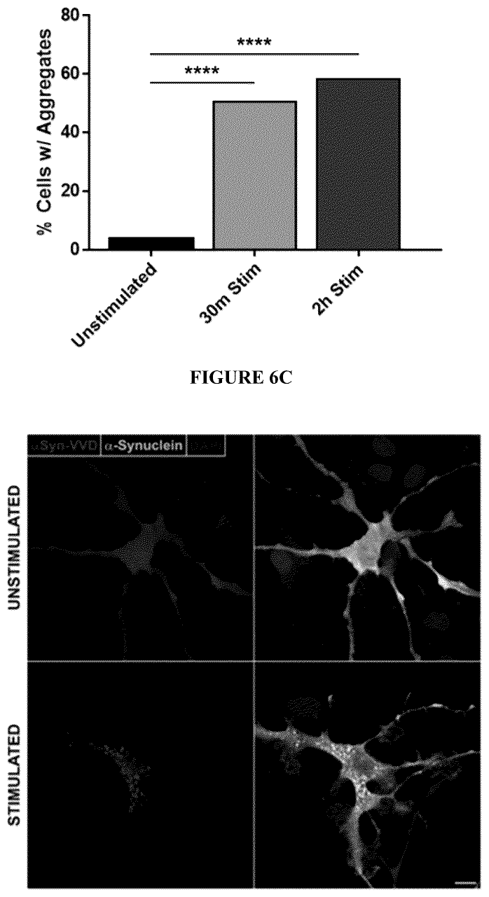

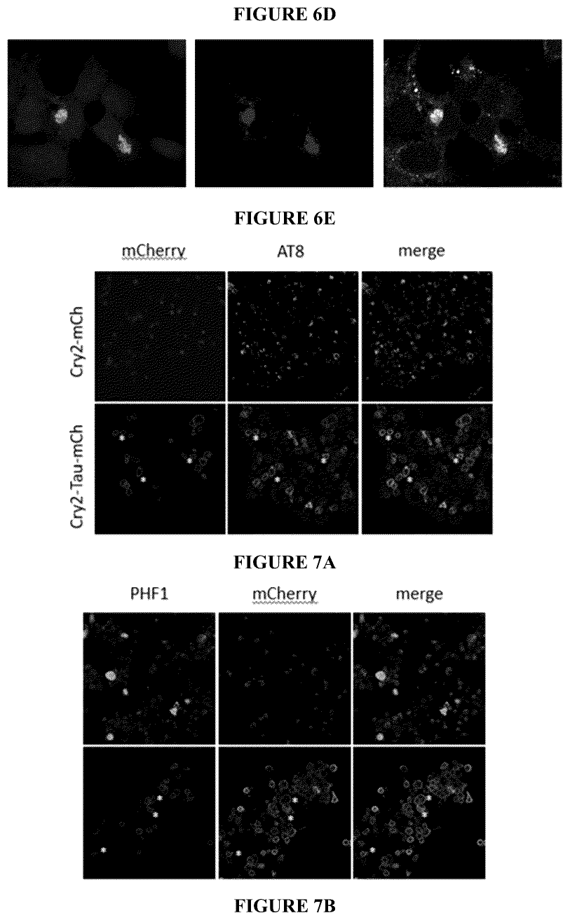

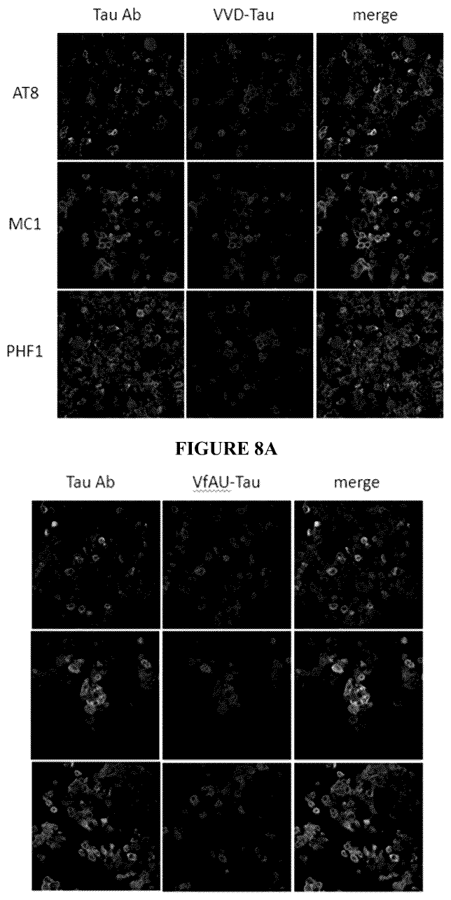

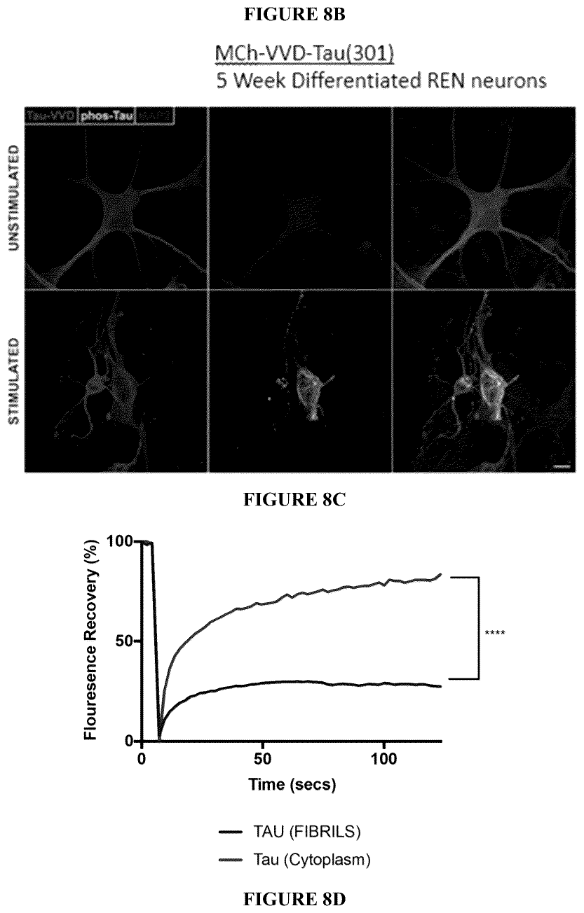

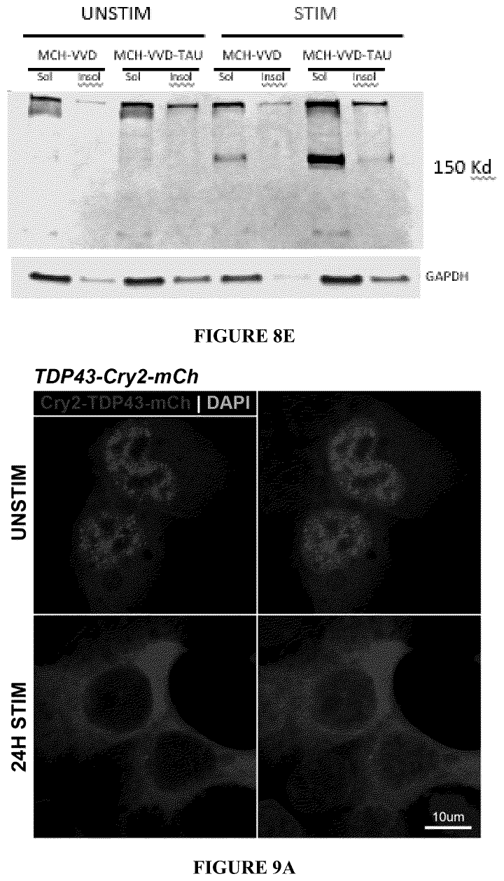

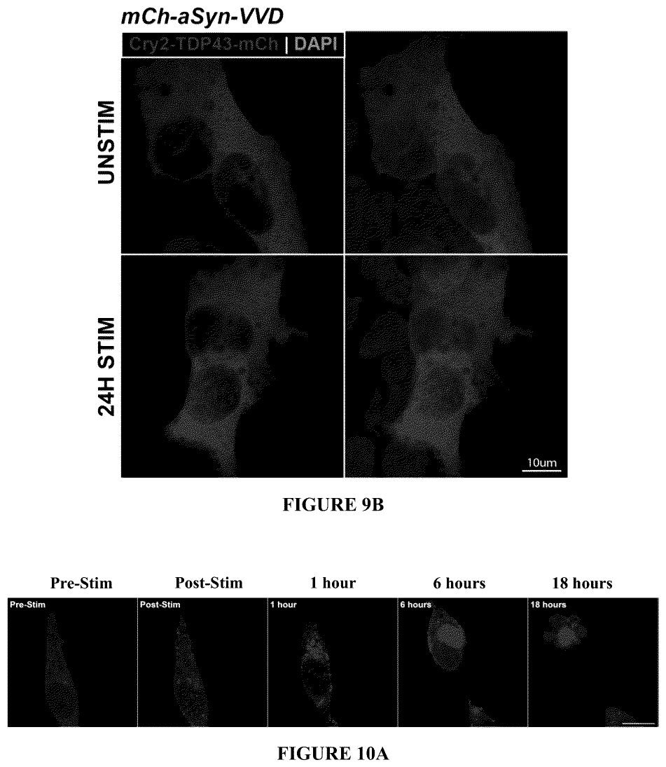

Vivid (VVD) is a protein generated in the Neurospora organism and is a photoreceptor that homodimerizes in response to blue light (within the 405-499 nm range). The Light-Oxygen-Voltage-Sensing (LOV) domain is common throughout many organisms but this domain is very small in this organism. NcLOV is only the LOV domain of the VVD protein but is conserved throughout other species. NcVVD comprises a small fragment of the N-term VVD protein and the VVD LOV domain. NcVVDY50W from the NcVVD protein sequence however is altered by a Y50W change that promotes a slower ability to dissipate from the dimerized state. It was shown here that persistent dimerization of LOV domains induces oligomerization with chronic light and when fused to an LCD containing protein (FIGS. 6, 8). Although LOV domains are not reported to form oligomers, a light stimulation paradigm to induce protein clustering of specific protein arrangements of these photoreceptors and specific target neurodegenerative disease proteins has been developed herein. Additional LOV domains used herein also include VfAU1, YtvA, EL222, RsLOV, and AsLOV2.

TABLE 3
Target neurodegenerative disease proteins.

| Gene | Encoded | Associated | Human | Predominant Cellular |
|---|---|---|---|---|
| Symbol | Protein | Disease | RefSeqGene | Localization |
| TARDBP | TDP-43 | ALS, FTD, AD, CTE | NG_008734.1 | Nuclear |
| SNCA | Alpha Synuclein | PD, LBD, AD, MSA | NG_011851.1 | Cytoplasmic |
| MAPT | Tau | AD, FTD, CTE, PD | NG_007398.1 | Cytoplasmic |
| FUS | FUS | FTD, ALS | NG_012889.2 | Nuclear |
| SOD1 | SOD1 | ALS | NG_008689.1 | Cytoplasmic |
| TIA1 | TIA1 | ALS | NG_029967.1 | Cytoplasmic & |
| | | | | Nuclear |
| ATXN2 | Ataxin 2 | ALS | NG_011572.2 | Cytoplasmic |
| HNRNPA1 | hnRNPA1 | ALS | NG_033830.1 | Nuclear |
| HNRNPA2B1 | hnRNPA2B1 | ALS | NG_029680.1 | Nuclear |
| EWSR1 | EWS RNA Binding | ALS | NG_023240.1 | Nuclear |
| | Protein 1 | | | |
| TAF15 | TATA box binding | ALS | NG_023279.1 | Nuclear |
| | protein factor 15 | | | |

Abbreviations:
ALS, Amyotrophic Lateral Sclerosis;
FTD, Frontotemporal Dementia;
PD, Parkinsons Disease;
AD, Alzheimer's Disease;
CTE, Chronic Traumatic Encephalopathy;
LBD, Lewy Body Dementia

Table 3 describes a list of target neurodegenerative disease proteins to generate protein arrangements that recapitulate neurodegenerative disease pathology. Novel arrangements of the photoreceptors in Table 2 fused to neurodegenerative disease proteins such as those listed in Table 3 that respond to these various blue light exposure paradigms to recapitulate neurodegenerative disease pathologies are disclosed herein. This technology is also used to aggregate the prion-like domains/LCD/IDDs of truncated versions of these components that comprise neuropathological aggregates in patients. In this example, studies have been performed with CRY2OLIG and LOV fused to TDP-43, α-synuclein, and Tau proteins. In another example, the target neurodegenerative disease protein can be Huntingtin gene/protein (Accession Ref. NM_002111; NP_002102.4). However, a method was developed that can be applied to any photoreceptor with dimerizing or oligomerization capabilities due to the light treatment paradigms created. The primary purpose of this method is to control the local concentration of neurodegenerative disease proteins for specific periods of time forcing intramolecular crowding of proteins that contain LCDs and aggregate in neurodegenerative diseases. Each neurodegenerative disease protein and photoreceptor arrangement can require specific light stimulation parameters due to the nature of the protein. For example, cytoplasmic proteins or truncated versions of nuclear proteins to localize them to the cytoplasm typically (but not always) require short stimulation paradigms while predominantly nuclear proteins chronic stimulation since the protein will need to be trapped in the cytoplasm during translation (e.g. TDP-43, FUS). Details of the stimulation paradigms are discussed below.

Light exposure then forces these unique fusion protein arrangements into close proximity and employing chronic or repeated light stimulation, neurodegenerative disease aggregate pathologies of full length proteins or the LCDs alone can be obtained. This temporal and spatial control of neurodegenerative disease aggregates is novel since it does not require overexpression or mutation that are only relevant to a small subset of patients for each disease (Table 1). These proteins are forced to interact and form the disease pathology as occurs in human patients rather than filling the cell with these aggregate prone proteins.

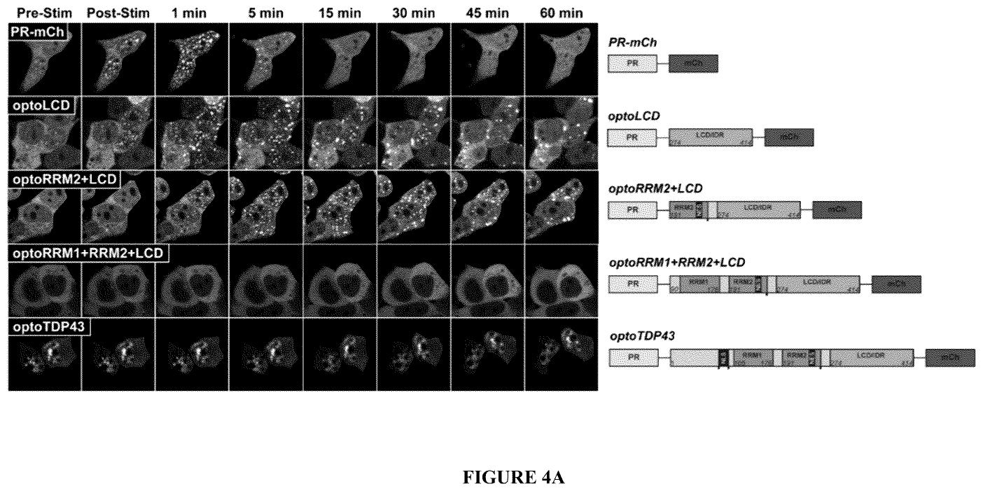

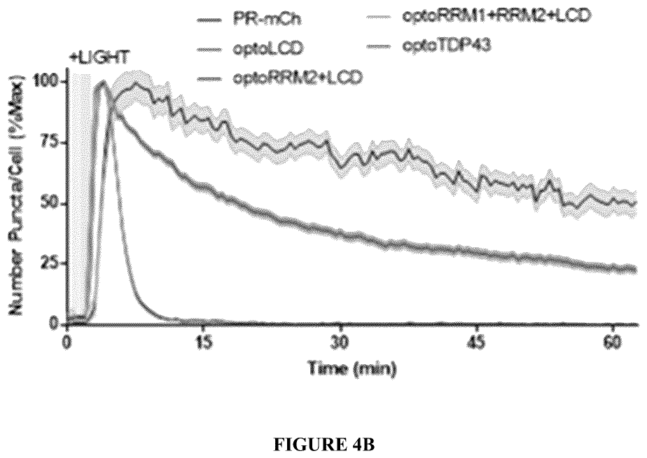

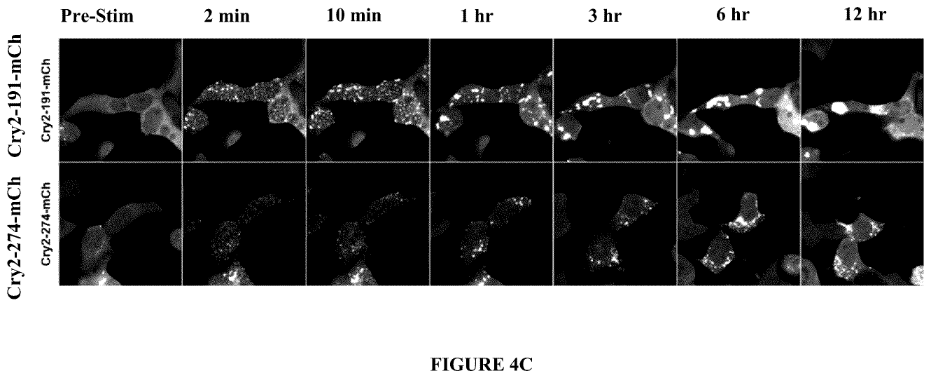

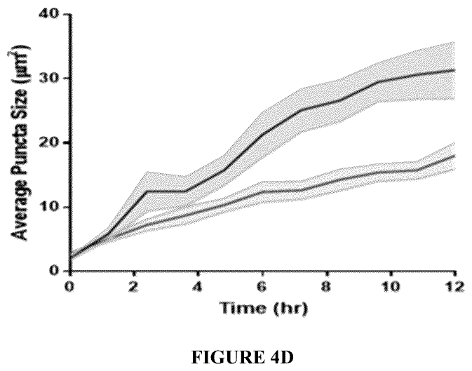

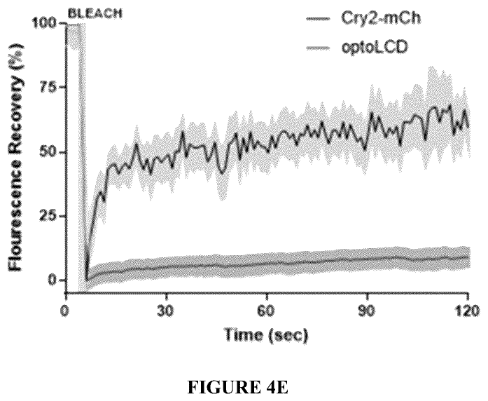

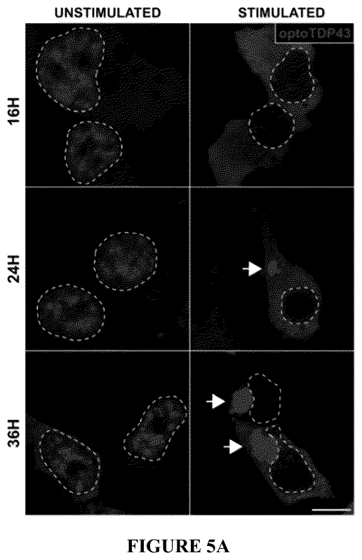

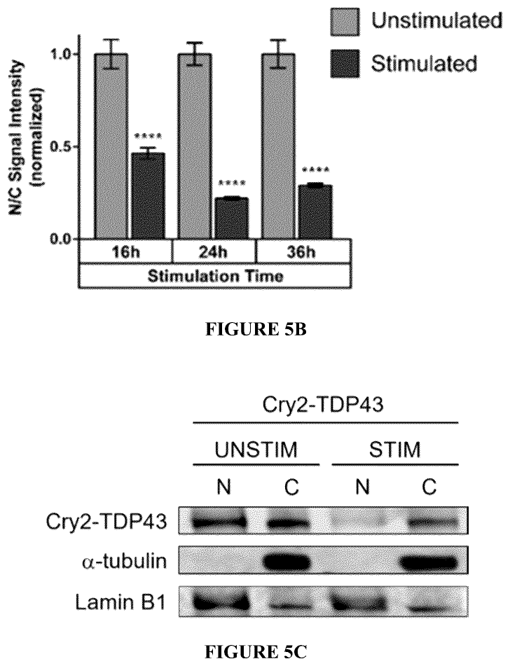

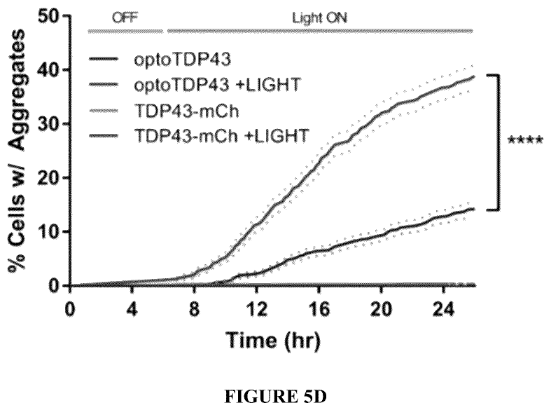

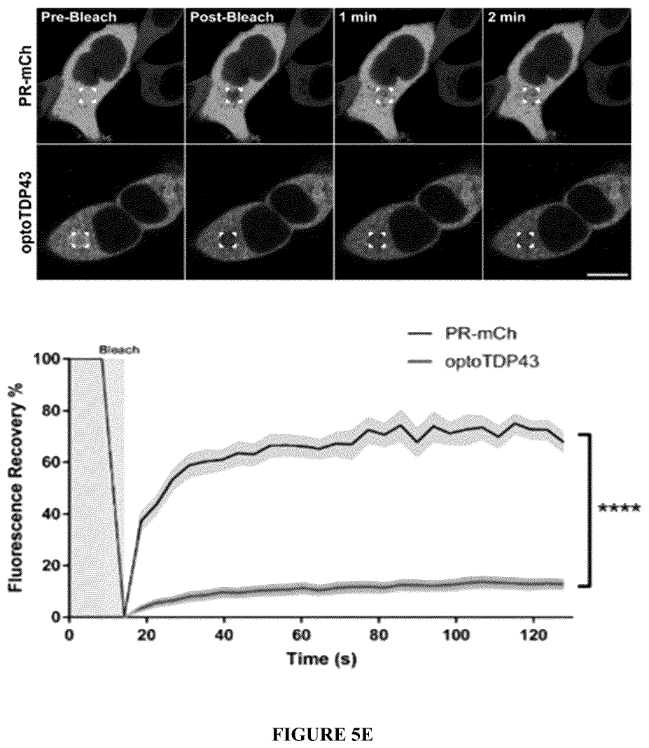

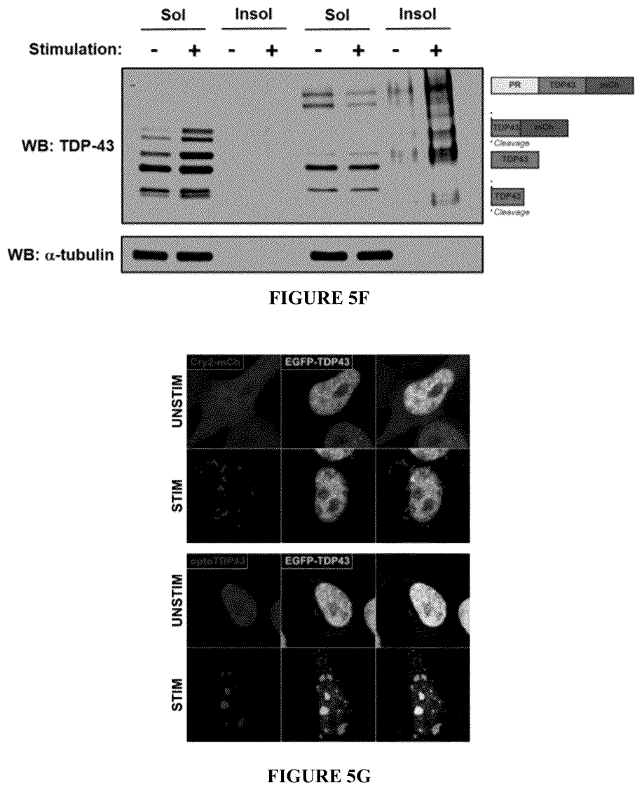

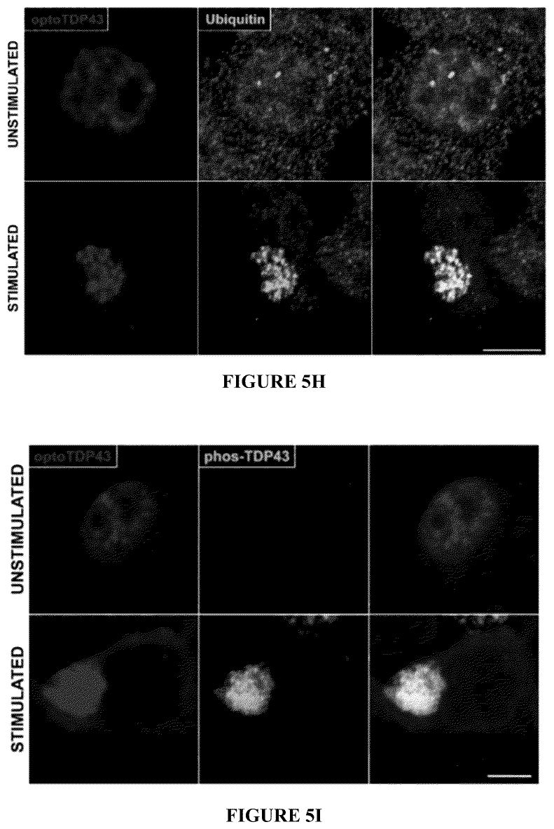

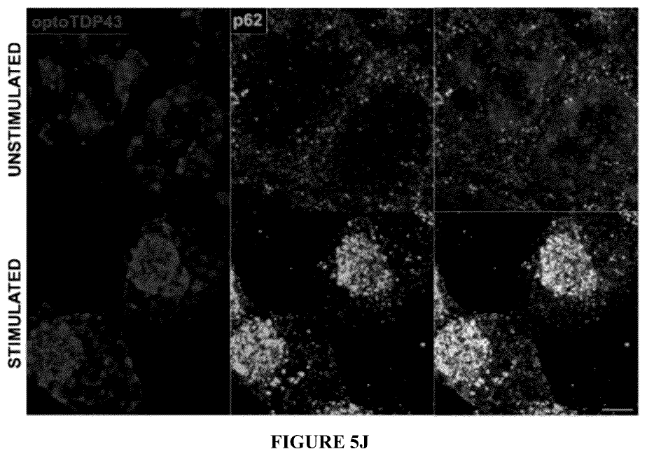

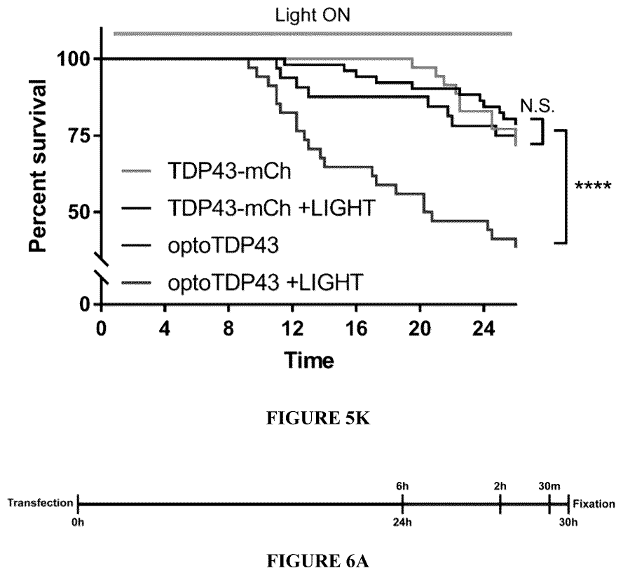

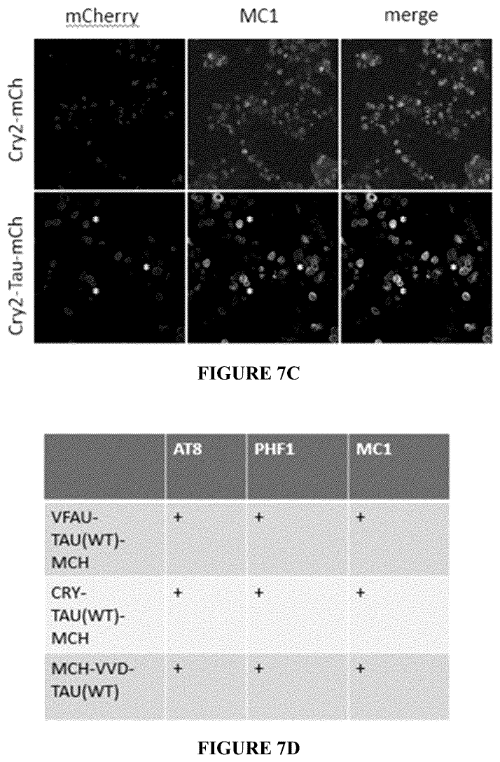

Beyond protein aggregation, this system creates light-induced pathologies that mimic key pathological features found in patients making it unique compared to overexpression systems. The TDP-43 protein, which contains an LCD, was combined with the CRY2OLIG photoreceptor domain which clusters when exposed to blue light and becomes insoluble and aggregated with persistent light treatment. Fusion proteins of the full length and partial LCD sequence were generated to recapitulate human neurodegenerative disease pathology, including the cytoplasmic mislocalization of nuclear proteins that occurs in patients with ALS and FTD. This system could induce neurodegenerative disease protein aggregates in live HEK cells by exposing them to various blue light stimuli paradigms (FIGS. 4-5). In addition, biochemical hallmarks of ALS, FTD, and AD (FIG. 7) were produced showing that the cell is responding to the light-induced aggregates as observed in patient CNS despite the addition of these tags. Notably, TDP-43 neuropathology is one of the more complex neuropathologies to mimic since it is a predominantly nuclear protein but is found to be mislocalized to the cytoplasm in patients. Employing this DNA arrangement and light stimulation system as an example, cytoplasmic TDP-43 aggregates were obtained that are also ubiquitinylated, cleaved, and hyperphosphorylated as observed in ALS, FTD, and AD patients. TDP-43 pathology is also found in AD as well as Chronic Traumatic Encephalopathy (CTE) thus highlighting the potential disease relevance of this work. In addition, a protein arrangement and light stimulation paradigm was developed to induce the aggregation of α-synuclein (FIG. 6), which is found in the CNS of patients diagnosed with Parkinson's Disease and Lewy Body Dementia. Methods were also developed to induce intracellular aggregation of Tau protein using both the Cry-PHR and LOV photoreceptors (FIG. 7) and persistent blue light stimulation. These tau tangles are pathological hallmarks of AD, FTD and CTE in HEK cells. These also exhibit pathological hallmarks of Tauopathy observed in patients including hyperphosphorylation using specific antibodies developed against pathological tau inclusions (FIGS. 7-8). Finally, it was shown that this method can be utilized to seed aggregations, as aggregates of TDP-43 formed using this method recruit endogenous TDP-43 to seed the pathogenic neuropathology (FIGS. 5F, 5G).

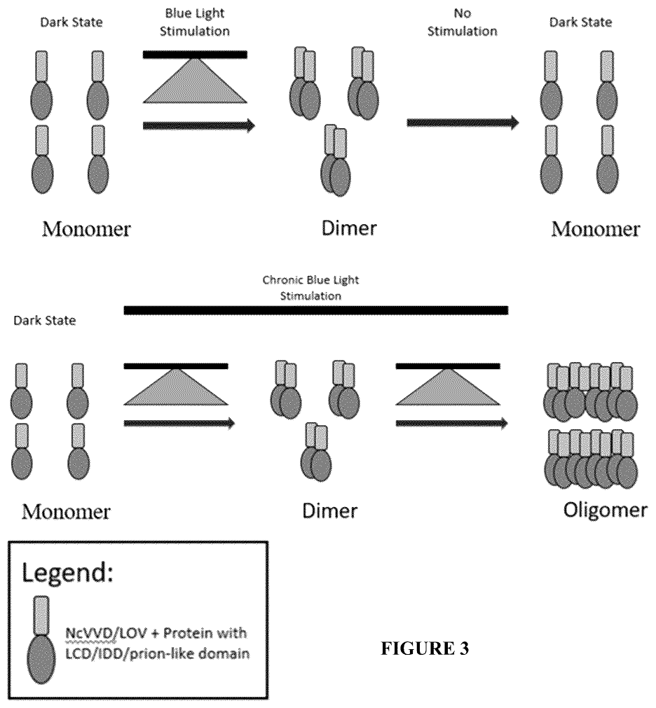

A novel methodology to force oligomerization and aggregation of LOV domain proteins fused to proteins that contain LCDs was also developed. The LOV domains, including NcVVDY50W has only been shown to dimerize with blue light stimulation. Using a chronic blue light stimulation paradigm (described in FIGS. 3 and 6-8), oligomerization and eventual aggregation of a LCD containing protein fused to the LOV sequence is induced. This amino acid sequence is significantly smaller than the PHR domain of CRY2 and may act as an alternative to CRY2 PHR since it is less likely to interfere with the endogenous target protein function.

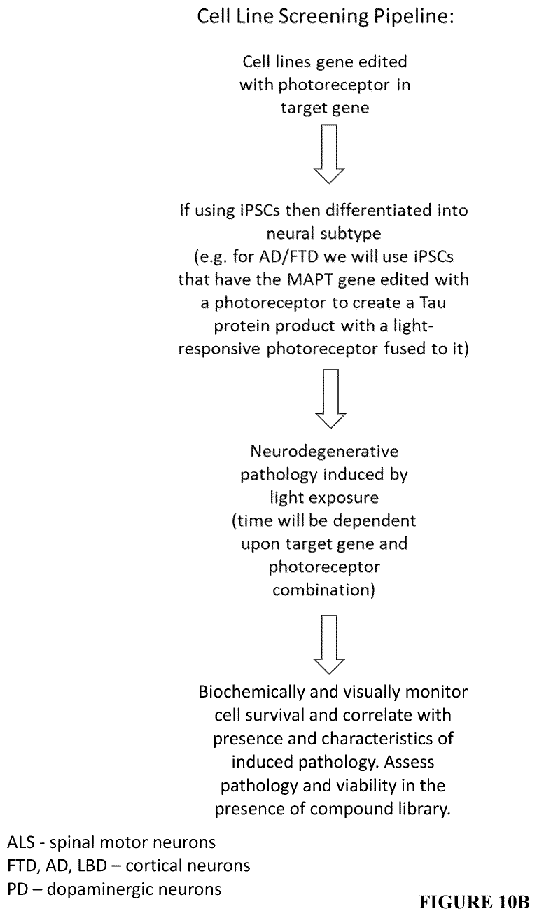

In some embodiments, DNA arrangements are constructed that encode for the PHR domains (CRY2PHR or CRY2OLIG) or LOV photoreceptor proteins (NcVVD, NcVVDY50W, Vfau1, YtvA, EL222, RsLOV, AsLOV2) (or 90% similarity) fused to either the LCD fragment or full length neurodegenerative disease proteins listed in Table 3 (or 90% similarity). In some embodiments, disclosed herein are methods of employing blue light exposure treatment paradigms to induce these neurodegenerative disease pathologies. In other embodiments, the resulting protein aggregates and cell viability are used as a readout for neurodegenerative disease drug screening (FIG. 10). This system is used by pharmacologically or genetically mitigating neurodegenerative disease pathology formation or resident time can identify novel compounds for the treatment of specific neurodegenerative diseases. The following embodiments are also disclosed:
1. Generation of Novel Model Systems: These photoreceptor sequences are inserted into the genome of various in vitro and in vivo systems which can act as a new model to study neurodegenerative diseases. Some examples of in vitro uses include human and rodent cell lines, induced pluripotent stem cells (iPSCs), or yeast. Some examples of in vivo uses include invertebrates: Drosophila melanogaster (fruit fly), Caenorhabditis elegans (round worm), or Danio rerio (zebrafish). Other examples of in vivo uses include vertebrates: mouse, rat, or non-human primate.
2. These compounds, methods, and systems (such as iPSC) with edited genomes are used in high throughput drug screening systems. To achieve this, neurodegenerative disease pathologies are induced by stimulating cells with light. Cell viability and formation and residence time of protein aggregate/pathology in the presence of compound libraries are then examined. In addition, assays also involve employing survival and neuropathology of in vivo models following induction with light.

### Example 2

### Materials and Methods

Plasmid Generation. All protein expression plasmids generated in this work were constructed using restriction enzyme (NEB) digested backbone vectors and PCR-generated DNA fragments (Q5® High-Fidelity Polymerase, NEB) assembled with Gibson Assembly (HiFi DNA Assembly Master Mix, NEB). To generate DNA expression constructs that allow for the option of both transient and stable protein expression and doxycycline-inducible expression, a complete bi-directional, TET-ON® 3G sequence from (pCMV-Tet3G (Clontech) was assembled within 5′ and 3′ PiggyBac transposon-specific inverted terminal repeat sequences (ITR) of the PiggyBac(PB)-CAG-eGFP plasmid using EcoRV and SpeI restriction sites. All plasmids were constructed using the expression vector PB-pCMV-Tet3G (PB-Tet3G) by PCR fragment assembly using Ecor1 and Not1 restriction enzyme sites. An N-terminal truncated version of VVD (residues 37-186, 17.1 kDa) (Addgene #58689) was used for these studies. VVD mutations (I74V, I85V) were introduced by site-directed mutagenesis with Gibson Assembly to generate the VVD (fast) sequence. The addition of a V5 tag sequence to tau (2N4R) and VVD (fast) plasmids was constructed using PCR-generated fragments from 2N4R tau (Addgene #92204) and V5 (Addgene #107596) and inserted into the PB-pCMV-TET3G plasmid to generate tau (tau(2N4R))-V5, and VVD (I74V, I85V (fast))-V5. The following optogenetic plasmids: VVD-Tau(2N4R), Tau(2N4R)-VVD-V5, Cry2olig-Tau(2N4R), and Tau(2N4R)-Cry2olig, were constructed using PCR-generated fragments from the VVD plasmid and cryptochrome 2 (Cry2Olig) (Addgene #60032) plasmids and inserted into the described PB-Tet3G plasmid. Additional optoTAU isoforms (1N4R, 0N4R) were constructed using the original tau(2N4R)-VVD-V5 sequence and two-fragment Gibson assembly to remove one or both N-terminal tau inserts and insert fragments simultaneously into EcoR1 and Not1 sites of the PB-Tct3G plasmid. OptoTAU point mutations (\hat{ }K280) and (\hat{ }K280, I277P, I308P) were generated through site-directed mutagenesis with Gibson assembly and inserted into the PB-pCMV-TET3G plasmid. OptoTAU-Halo was generated using a pCMV-HALO-TAG® plasmid to construct tau(2N4R)-VVD-V5-Halo into the PB-Tet3G plasmid. All plasmids were confirmed by Sanger sequencing (Genewiz).

HEK293 cell culture, transient transfection, and stable cell line generation. HEK293 cells were purchased from (ATCC). The cells were maintained in Dulbecco's Modified Eagle Medium (DMEM/F12) supplemented with 10% fetal bovine serum at 37° C. and 5% CO2. On day 1, cells were seeded in 6-well plates at 70-80% confluency. On day 2, at >90% confluency, cells were transfected with cDNA using X-tremeGENE HP DNA (Millipore Sigma) according to the manufacturer's instructions. On day 3, the cell culture medium (1.5 ml for 6-well, and 500 μl for 24-well plates) was replaced with phenol-free DMEM/F12 containing 1 μg/ml doxycycline (Millipore Sigma) and placed in a dark incubator for 24 hours. On day 4, the cells were incubated in the presence or absence of blue light exposure for the indicated times. For the generation of HEK293 cells that stably express Tau-VVD-V5-Halo, cells were seeded on 6-well plates at 80% confluency. The following day, cells were co-transfected with PB-Tet3G-Tau-VVD-V5-Halo plasmid (2 μg DNA; transposon) and the PBase enzyme (transposase) expression plasmid (0.5 μg DNA) at a 4:1 transposon:transposase ratio using X-tremeGENE HP DNA. On the following day, fresh medium containing 5 μg/ml puromycin (Millipore Sigma) was added to the cell culture medium. Once confluent, the cells were trypsinized in 0.25% Trypsin-EDTA (ThermoFisher) and plated in a T75 flask in the presence of 5 μg/ml puromycin selection for up to 2 weeks.

ReN cell culture, differentiation, and stable cell line generation. The RENCELL® VM (Millipore) neural progenitor cell (NPC) line was maintained and differentiated as previously described (40). Briefly, cells were maintained on MATRIGEL™-coated flasks in proliferation medium (DMEM/F12 supplemented with 1× B27 (ThermoFisher), 2 μg/ml heparin (Millipore Sigma), 20 ng/ml bFGF (Millipore Sigma), and 20 ng/ml hEGF (Millipore Sigma) and filtered through a 0.2 μm PES filter (Fisher Scientific)). For stable expression of PB-Tet3G-Tau-VVD-V5-Halo, and PB-Tet3G-VVD-V5, cells were seeded on 6-well MATRIGEL™-coated plates at 80% confluency in proliferation medium. On day 2, cells were co-transfected with appropriate PB-Tet3G plasmid (2 μg DNA) and the PBase (500 μg DNA) using the JETOPTIMUS® (Polyplus transfection) transfection reagent according to the manufacturer's instructions. On day 3, fresh proliferation medium supplemented with 1 μg/ml puromycin was added to the cell culture medium. When the cells reached confluency, they were treated with Dispase (1 U/ml, StemCell Technologies), scraped, and transferred to T75 flasks in proliferation medium supplemented with 1 μg/ml puromycin for 2 weeks.

Differentiation. RENCELL® VM neural progenitor cells were seeded on MATRIGEL™ coated 6-well plates, 24-well coverslips, or 24-well glass imaging plates in differentiation medium (DMEM/F12 supplemented with 1× B27 and 2 μg/ml heparin and filtered through a 0.2 μm filter). Cells were allowed to differentiate for 25-days with medium changes every three days.

Peptide Inhibitor preparation. The previously described W-MINK inhibitor with the following amino acid sequence (DVWMINKKRK, SEQ ID NO:106) was synthesized by Genscript with a minimum purity of 90%. The stock peptide was dissolved in sterile DPBS to a final stock concentration of 5 mM, aliquoted for single use, and stored at −20° C. For each experiment, W-MINK was diluted to 10 μM in phenol red-free medium.

Blue light stimulation. Chronic blue light exposure was performed in 24- or 6-well plates using a custom-built LED stage (5 μM, 465 nm) housed in a cell culture incubator (37° C. and 5% CO₂). For multi-day Confocal imaging experiments, light-exposed cells were transferred from the cell culture incubator to a preheated (37° C. and 5% CO₂) microscope stage top incubator and allowed to equilibrate for 10 min prior to imaging. For the 16 hour time-lapse imaging experiments, a custom-built LED stage (5 μM, 465 nm) designed for the Confocal stage top incubator allowed simultaneous light exposure and imaging. A dark cell culture incubator was used for the control dark condition.

LDH cytotoxicity assay in HEK293 and ReN cells. Cell culture medium was collected from 6-well plates after the respective blue light exposure experiment. For each experimental condition, 50 μl of the cell culture medium was added to 3 wells of a 96-well plate to perform the lactate dehydrogenase (LDH) release assay using the CYQUANT™ LDH Cytotoxicity assay kit (ThermoScientific) according to the manufacturer's instructions. Briefly, 50 μl of the Reaction Mixture was added to each sample. The plate was protected from light and incubated for 30 min at room temperature (RT). 50 μl of Stop Solution was then added to each well. The absorbance was measured at 490 nm and 680 nm using a Synergy microplate reader (Biotek). To determine LDH activity, the 680 nm absorbance value (background signal) was subtracted from the 490 nm absorbance value (LDH signal).

Detergent solubility assay (SarkoSpin). All detergent solubility assays were performed from HEK293 and ReN cells plated on 6-well dishes. Detergent solubility assays were performed according to the SarkoSpin method with minor modifications. For the stable optoTau-Halo HEK293 cell line and transient transfection experiments, cells were washed with RT DPBS following removal of the cell culture medium. The cells were then scraped in 170 μl of RT 1× homogenization-solubilization (HS) buffer (10 mM Tris, pH7.5, 150 mM NaCl, 0.1 mM EDTA, 1 mM dithiothritol, complete EDTA-free protease inhibitors (Roche) and phosphatase inhibitors (Millipore Sigma), and 0.5% Sarkosyl), followed by the addition of a mixture of 2 mM MgCl2 and 25 U (per well) Benzonase (Millipore Sigma) before cell scraping. For sample solubilization, 52 μl 1× HS buffer (no Sarkosyl) and 178 μl 2× HS buffer with 4% Sarkosyl were added to aliquots of the 170 μl lysate for a final concentration of 2% sarkosyl in 400 μl of lysate. Alternatively, for ReN cell cultures, three wells from a 6-well plate were combined in a total of 400 μl of lysis buffer. After a 3-minute incubation at RT with vortexing every 10 minutes, the samples were diluted by adding 200 μl of 1× HS buffer. For fractionation, the lysate was centrifuged at 21,200 g on a benchtop centrifuge (Eppendorf) for 45 minutes at RT. Supernatants (soluble fractions) were collected in a fresh tube. The insoluble pellet was washed with 250 μl of wash buffer (1× HS buffer, 1.5% Sarkosyl). The pellet was briefly vortexed and then centrifuged at 21,200 g for 30 minutes at RT. The wash buffer was completely removed prior to freezing the insoluble pellet. Both the soluble fraction and insoluble pellet were frozen (−20° C.) prior to Western blot analysis.

Western blot analysis. For gel loading, the soluble fractions were mixed with loading 389 sample buffer (4× BOLT™ LDS Sample buffer (ThermoFisher) containing 4% β-mercaptoethanol (BioRad). Insoluble pellets were resuspended in 30 μl of 1× HS buffer containing 0.5% Sarkosyl and 10 μl of loading sample buffer (4× BOLT™ LDS Sample buffer (ThermoScientific). The samples were then heated for 10 min at 70° C. Soluble and insoluble (20 μl, 50% of total pellet) samples were loaded onto NUPAGE™ 3 to 8% Tris-Acetate, 1.0 mm gels (ThermoFisher). The samples were run at 50V for 1 h followed by 100V for 1 h using Novex NuPAGE Tris-Acetate SDS Running Buffer (ThermoFisher). Gels were equilibrated in fresh Towbin transfer buffer (25 mM Tris-base, 192 mM glycine, 20% (v/v) ethanol, 0.05% SDS (pH8.3)) for 10 min. Protein was then transferred to 0.45 μm nitrocellulose membranes (BioRad) using a wet transfer method (INVITROGEN™ Mini Blot Module) at 10V for 90 min. After the transfer, the membranes were rinsed for 5 min in distilled water, stained with Ponceau S (0.1% (w/v) in 5% acetic acid; Sigma Aldrich) for 5 min, and rinsed with distilled water to remove excess Ponceau S. For total protein measurement, Ponceau S-stained membranes were imaged on an Amersham ImageQuant 800 imager (GE Healthcare). Membranes were then rinsed for 10 min and blocked in 1× TBS (Tris-buffered Saline)+5% non-fat milk powder for 30 min at RT, and then briefly rinsed with 1× TBS-T (1× TBS+0.1% Tween 20) and incubated with primary antibodies diluted in 1× TBS-T+5% milk overnight at 4° C. on a shaker. Membranes were washed with 1× TBS-T (3×10 min) and then incubated with a species-specific IgG (H+L) HRP-conjugated secondary antibody (BioRad) in 1× TBS-T, 5% milk for 1 hour at RT. Membranes are then washed with 1× TBS-T (3×10 min) and 1× TBS (3×5 min). Western blots were developed using Western Lightning Plus-ECL (PerkinElmer) or SUPERSIGNAL™ West Femto substrate (Fisher Scientific) and imaged on an Amersham ImageQuant 800 imager. Quantification was performed using ImageLab software 6 (BioRad). For both soluble and insoluble fractions, background-adjusted band densities were obtained for the total tau monomer (75 kDa) and the total HMW tau signal (>100 kDa). For soluble fractions, tau protein was normalized to total protein (Ponceau S), and a HMW/Monomer tau ratio was calculated from a single non-overexposed image. All HEK293 data presented was from at least n=3 independent biological experiments using two-technical replicates per experimental condition. All REN data presented is from at least n=3 independent biological experiments with three technical replicates/wells pooled together during cell processing.

Immunocytochemical analysis. Subcellular distribution and colocalization of optoTau(2N4R)-V5-Halo with tau antibodies and amyloid dyes were investigated by multi-labeling immunocytochemical analysis of fixed optoTau-V5-Halo expressing HEK293 cells or ReN cells grown on MATRIGEL™-coated coverslips. Following the respective experimental protocol, cells were rinsed with 1× DPBS at RT and then fixed with freshly made 4% paraformaldehyde (PFA) for 15 min at RT. Cells were washed with 1× DPBS (3×5 min) before incubation with a permeabilization/blocking solution (0.1% Triton X-100, 0.1% Tween 20, 5% normal goat serum in 1× DPBS) for 30 min (3×10 min) at RT. Cells were incubated with primary antibodies overnight at 4° C., washed with 1× DPBS (3×10 min) at RT, followed by incubation of Alexa Fluor-conjugated secondary antibodies for 1 h at RT in the dark. All antibodies were diluted in a permeabilization/blocking solution. After primary and secondary antibody incubations, cells were stained with the amyloid Amytracker 680 dye (1:1000; Ebba Biotech) for 30 min at RT followed by sequential washes with 50% ethanol/1× DPBS (2×3 min), 30% ethanol/1× DPBS (2×3 min), 10% ethanol/1× DPBS (3×1 min), and 1× DPBS (3×5 min). 4″6′-diamino-2-final-indol (DAPI), diluted in 1× DPBS, was then added to the cells for nuclei counterstaining followed by 1× DPBS washes (3×5 min). The coverslips were mounted with PROLONG® Diamond Antifade mounting medium (ThermoFisher) and stored in the dark. For fixed-cell HaloTag fluorescence imaging, the HaloTag-specific dye, Oregon Green (Promega), was added at 1 μM to phenol-free DMEM/F12 containing 1 μg/ml doxycycline for 24 hours before light stimulation followed by cell fixation, antibody/nuclei staining, and mounting as described above.

AggFluor live-cell imaging. OptoTau-V5-Halo expressing HEK293 cells were seeded on MATRIGEL™-coated 24-well glass-bottom plates at 80% confluency. For the 16 hour time lapse imaging experiments, on the day after plating the cells, the culture medium was removed, and the cells were incubated with DMEM/F12 supplemented with 1 μg/ml Hoechst dye (ThermoFisher) for 10 min. OptoTAU protein expression was then induced by refreshing the cell culture medium with DMEM/F12 containing the following components: 1 μg/ml doxycycline to induce protein expression, 10 μM cytosine β-Darabinofuranoside (AraC; Millipore Sigma) to mitotically arrest cells, and both AggFluor probes 1 μM P1h (red) and 1 μM P18h (green). For cells treated with W-MINK inhibitor, an aliquot of the above dye solutions was combined with a 10 μM W-MINK solution and added to cells. Cells were then placed in a dark incubator for 4 hours to allow protein expression and optoTau-V5-Halo labeling with AggFluor probes. Cells were then allowed to equilibrate on the preheated (37° C. and 5% CO₂) stage top incubator for 10 min prior to imaging. Two fields of view were followed for both untreated and treated cells. After the initial imaging of a pre-light timepoint (time=0 h), cells were exposed to light and imaged every two hours over a 16-hour experiment. For multi-day imaging, protein expression was induced by replacement with fresh DMEM/F12 containing the following components: 1 μg/ml doxycycline, 10 uM AraC, and both AggFluor probes 1 μM P1h and P18h or 0.5 μM coumarin (Blue) Halo-Tag ligand (Promega). For cells treated with the W-MINK inhibitor, an aliquot of the above dye solutions was combined with a 10 μM W-MINK solution and added to cells. Cells were then placed in a dark incubator for 24 hours to allow protein expression and optoTau (2N4R)-V5-Halo labeling with Halo-tag ligands. The culture medium was replaced with fresh DMEM/F12 to remove excess probes and placed in a dark incubator for 5 min. Medium was removed, and cells were incubated with DMEM/F12 supplemented with 1 μg/ml Hoechst for 10 min in a dark incubator. Then, the medium was replaced with fresh phenol-free DMEM/F12 containing lug/ml doxycycline and 10 μM AraC and incubated in the dark for 1 hour before live-cell imaging to capture pre-blue light conditions. After imaging, cells were treated with light for 24 hours after which cells were imaged to capture post-light conditions. After imaging, cells were placed under dark conditions for 24 hours and then imaged to capture a post 24 hour light/post 24 hour dark (aggregate persistence) condition. For stable optoTau-V5-Halo expressing ReN 25-day, differentiated cells had protein expression induced by replacement with fresh differentiation medium containing the following components: 1 μg/ml doxycycline and both AggFluor probes P1h and P18h or coumarin Halo-Tag. For cells treated with the W-MINK inhibitor, an aliquot of the above dye solutions was combined with a 10 μM W-MINK solution and added to cells. Thereafter, both ReN and HEK293 cells were treated identically except for the use of the appropriate cell culture medium.

Image acquisition. Confocal images were obtained with a Nikon A1R HD25 confocal microscope using a Galvano scanner. For fixed-cell imaging: n=6 fields of view per coverslip from at least n=3 independent biological experiments were taken from cells under either a 40× or 60× oil-immersion objective (either 512×512 or 1024×1024 frame size; 1.39× zoom; 1.2 Airy units pinhole dimension; unidirectional scanning; Offset unchanged) with appropriate negative controls (dox-free conditions with appropriate combinations either primary/secondary antibodies and Halo dyes). For every image, Z-stacks were performed with a step size of 0.15 μm allowing imaging of an entire layer of cells. Acquisition settings (laser intensity, gain, and offset) were kept constant for all images within a staining group. For live-cell imaging: All live-cell imaging experiments were performed on a Nikon A1R HD25 laser-scanning confocal microscope outfitted with a Tokai HIT stage top incubator using a resonant scanner. Cells were allowed to equilibrate on the preheated (37° C. and 5% CO2) stage top incubator for 10 min before imaging. For 16 hour timelapse imaging, n=2 fields of view from n=4 independent biological experiments were taken from cells under 60× oil-immersion objective (512×512 frame size; 1.39× zoom; 1.2 Airy units pinhole dimension; unidirectional scanning; Offset unchanged). For every image, we performed Z-stacks with a step size of 0.3 μm allowing imaging of an entire layer of cells. Acquisition settings (laser intensity, gain, and offset) were kept constant throughout the experiment. For multi-day imaging, n=6-10 fields of view per well (two wells per condition) were taken from cells under the 60× oil-immersion objective. (512×512 frame size; 1.39× zoom; 1.4 Airy units pinhole dimension; unidirectional scanning; Offset unchanged). For every image, Z-stacks were performed with a step size of 0.3 μm, allowing imaging of an entire layer of cells. Timing and order of image acquisition were alternated across experiments between experimental groups. Acquisition settings (laser intensity, gain, and offset) were kept constant for all time points during a multiday imaging experiment. For both fixed- and live-cell imaging, representative images of optoTau-V5-Halo expressing HEK293 or ReN cells were acquired with higher resolution settings (Nyquist sampling) using a 60× oil-immersion objective.

Image analysis. Confocal images were post-processed using Nikon 3-D deconvolution, and MaxIP images were further processed and analyzed using Nikon's NIS-Elements software. Intensity-based analysis was conducted using the General Analysis 3 (GA3) plugin (NIS-Elements). Before any quantification was assessed, all the channels collected within an image were corrected for background subtraction and potential uneven illumination using the “rolling ball” method. The general analysis included the counting of nuclei number (DAPI) per field of view and the total summed intensity (per cell or field of view) for the specific channels being imaged (FITC, TRITC, and Cy5).

Statistical analysis. Statistical significance was calculated with GraphPad Prism software (Version 9.3) and resulting p-values less than or equal to 0.05 were considered significant. No statistical methods were used to predetermine sample sizes. Normality of the data sets was verified with the Shapiro-Wilk normality test and equality of variances with an F test. Otherwise, data distribution was assumed to be normal. Unpaired Student t-tests were used to determine statistical significance in datasets comparing two variables. One-way or two-way ANOVA followed by Tukey's multiple comparison test was used for multiple variables. Data are presented as means±SEM. *p<0.05, **p<0.01, ***p<0.001, ****p<0.0001. For details of statistical analysis, see respective figure legends.

### Example 3

### Results

### Vivid (VVD) Provides Superior Light-Specific Control of Tau Aggregation

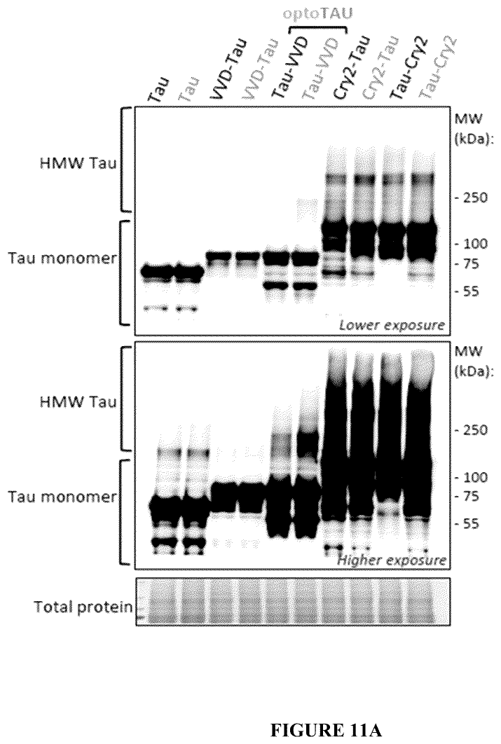

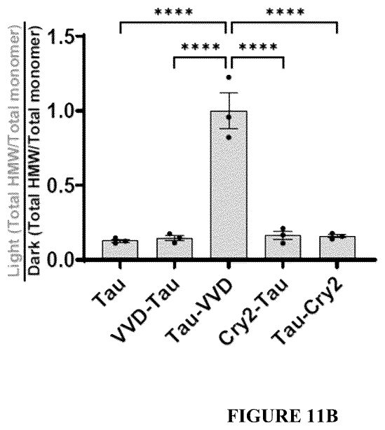

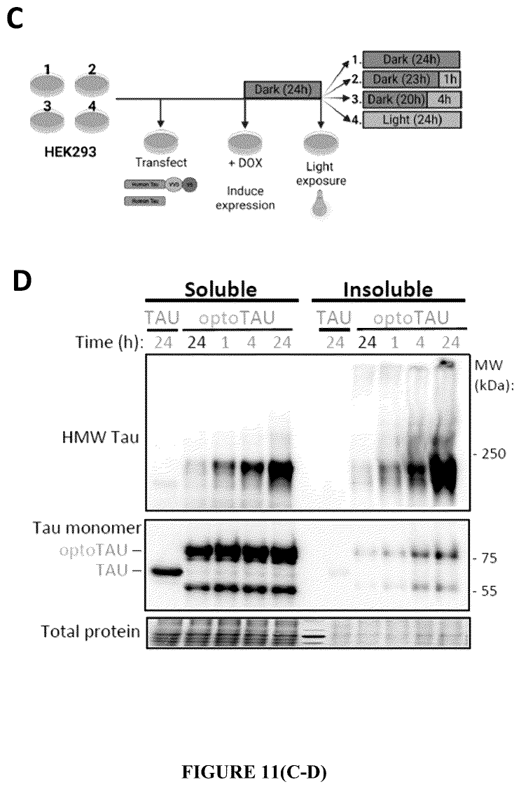

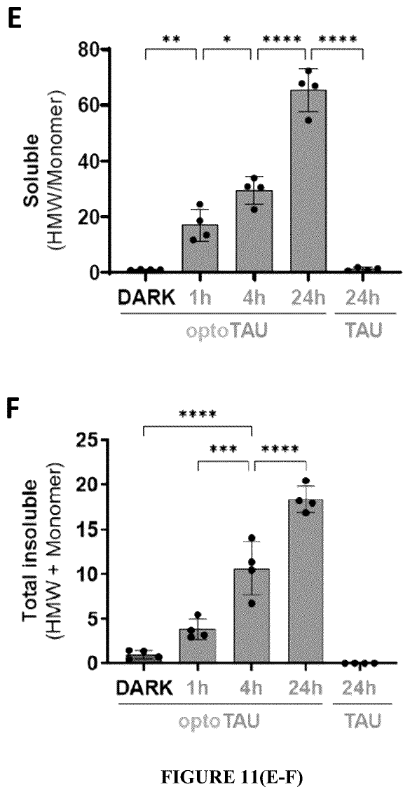

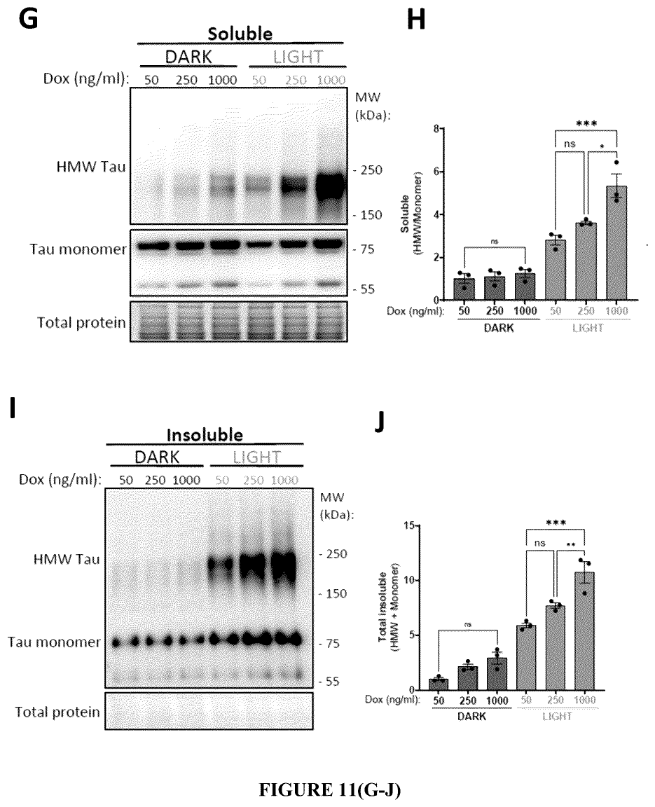

Vivid (VVD) is a small photoreceptor (approximately 17 kDa), derived from Neurospora crassa, that forms rapidly reversible, antiparallel homodimers upon light exposure. It was reasoned that the size (17 kDa) and dynamic dimerization properties of VVD would render it an invaluable tool to precisely control protein-protein interactions in living cells. To optimally induce VVD dimerization, two mutations were introduced in VVD (I74V, I85V; VVD (fast)), which allow rapid “switch-off” photo cycles that are critical for both the light-selective and reversible dimerization properties of VVD. First, the VVD (fast; I74V, I85V) mutant was introduced to the N- or C-terminus of full-length (2N4R) tau and a doxycycline (Dox)-inducible TET-ON 3G system was used to control the levels and temporal induction of tau expression. The C-terminal VVD tag (Tau-VVD), herein referred to as optoTAU, provided superior light-specific control of high molecular weight (HMW) tau formation relative to Tau-Cry2, Cry2-Tau, VVD-Tau, and untagged tau (negative control) (FIG. 11A-B).

### OptoTAU Induces Temporal and Concentration-Dependent Control of Tau Aggregation

Tauopathies are characterized by the gradual accumulation of detergent-insoluble protein aggregates. Accordingly, protein aggregation is generally described as a time- and concentration-dependent process that proceeds toward “irreversible” insolubility under pathological conditions. To determine whether optoTAU is a model of the time- and concentration-dependent aggregation of tau, light exposure time-course and concentration curve experiments were performed. OptoTAU displayed a gradual time- (FIG. 11C-F) and concentration-dependent (FIG. 11G-J) increase in Sarkosyl soluble HMW tau oligomers and insoluble tau aggregates. These findings strongly establish optoTAU as an advanced optogenetic model with light-specific temporal control of tau aggregation.

### AggFluors Permit Live-Cell Visualization of Tau Aggregation

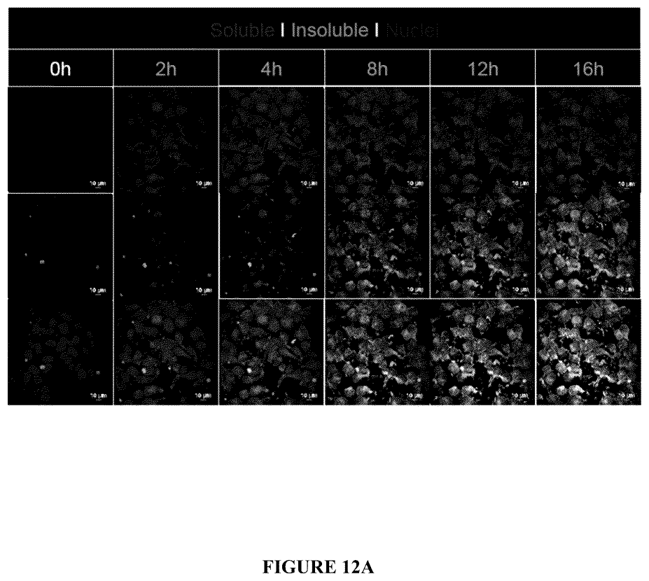

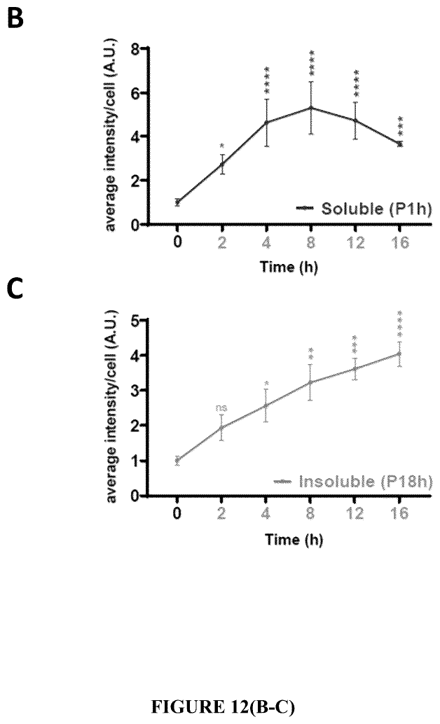

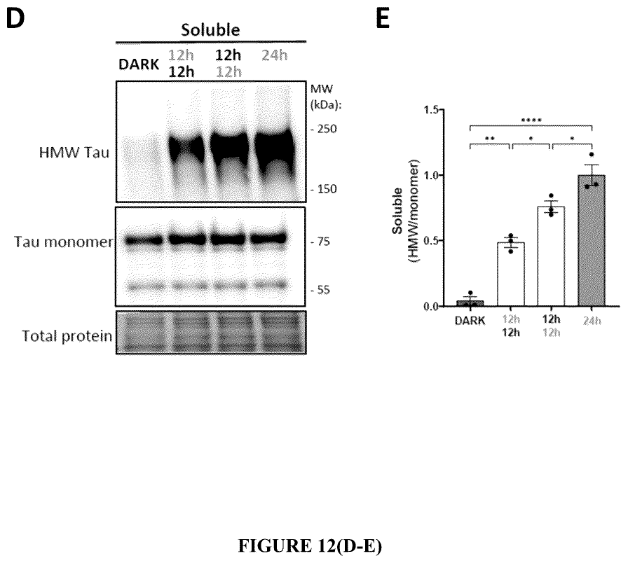

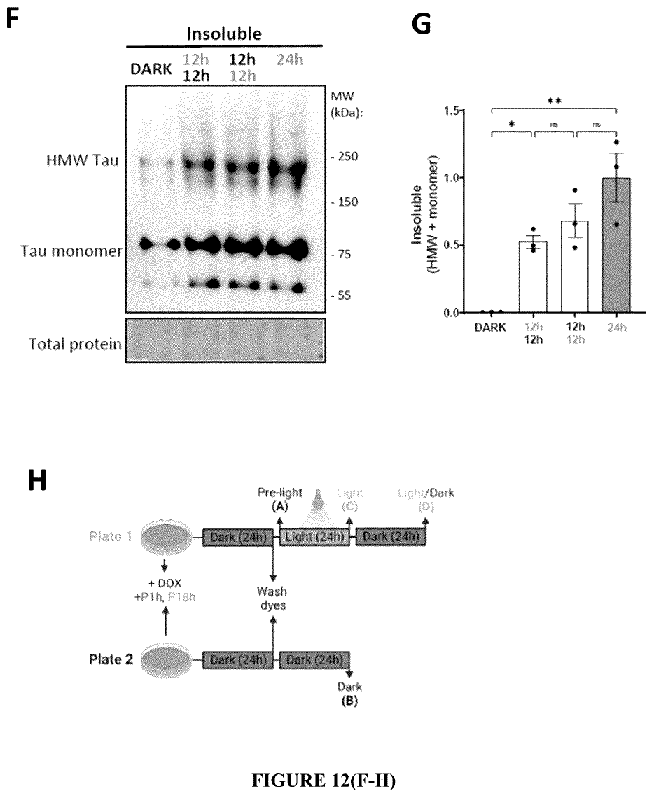

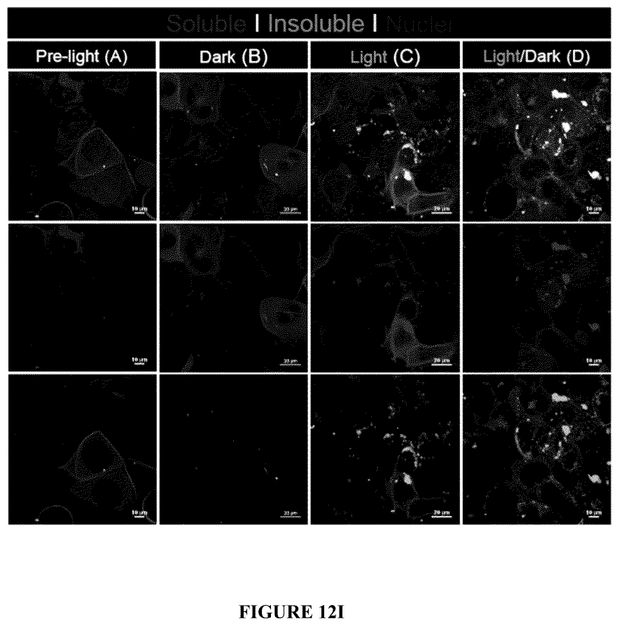

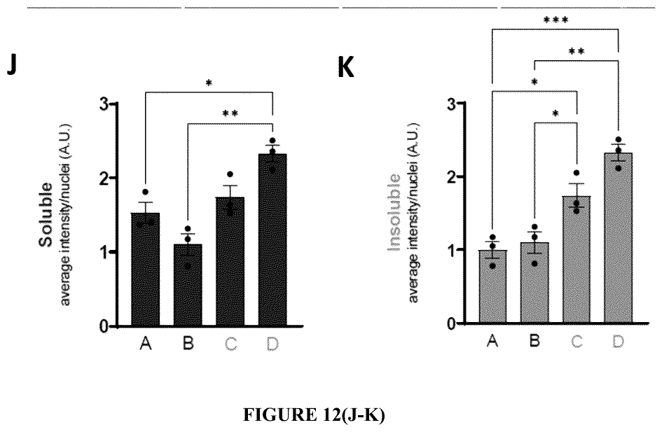

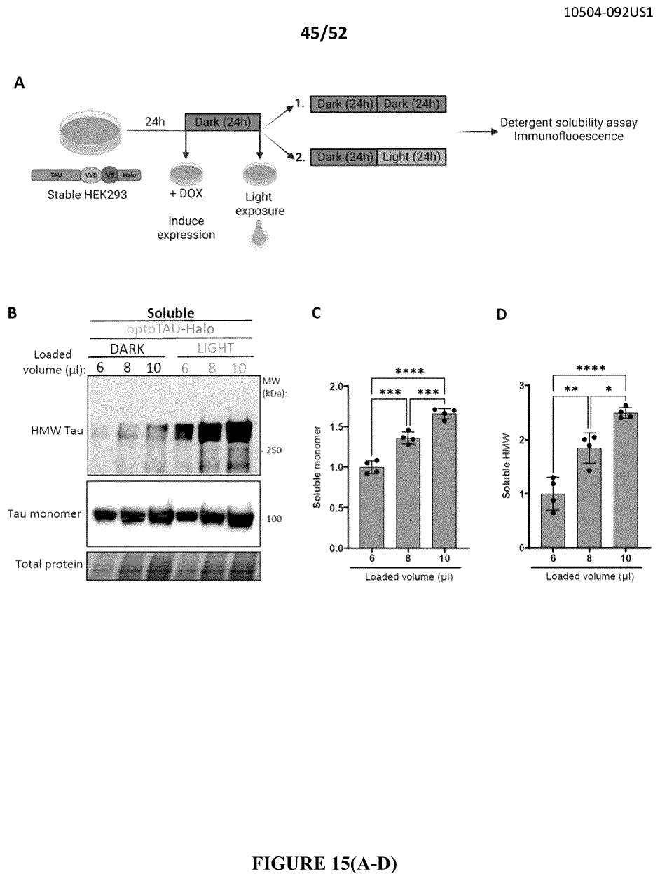

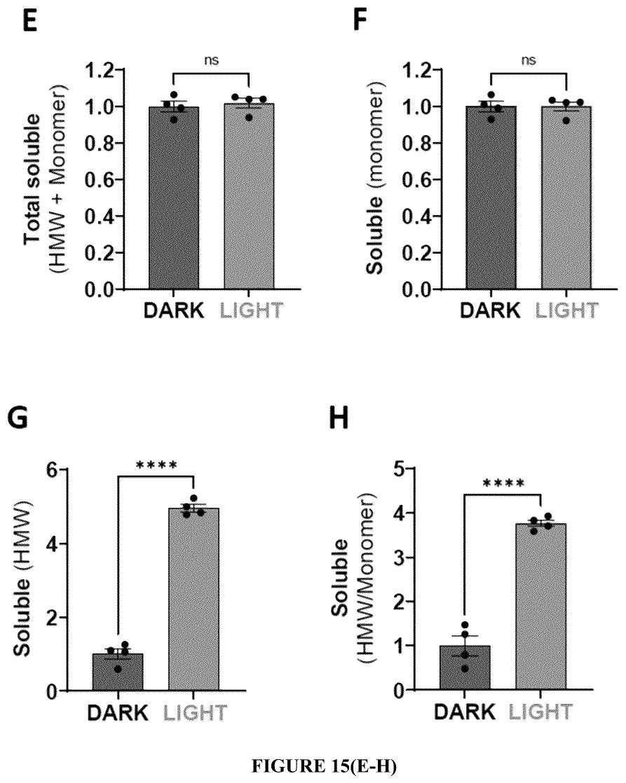

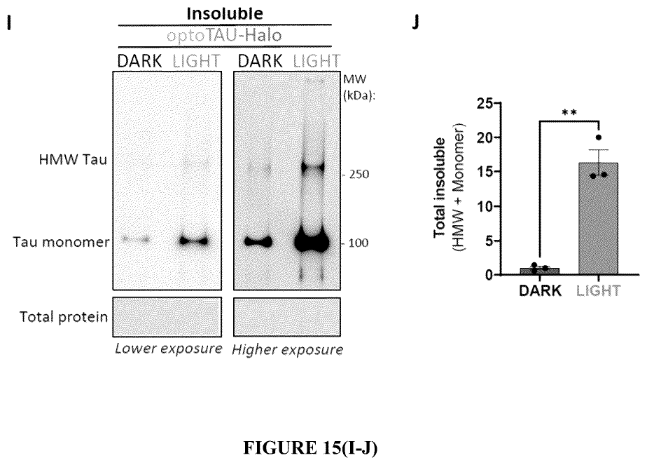

Prior to this discovery, no method existed to reliably visualize and distinguish between soluble HMW tau oligomers and insoluble tau aggregates in live cells. The optoTAU system was combined with the AggTag method for this purpose, which utilizes a self-labeling protein tag, e.g., HaloTag, and covalently-bound viscosity-sensitive fluorescent probes, i.e., AggFluors, that display quenched fluorescence until a folded protein begins to misfold and aggregate. As such, AggFluors with high rotational barriers fluoresce in lower viscosity environments (soluble oligomers) relative to fluorophores with low rotational barriers (insoluble aggregates), allowing live-cell visualization of protein aggregation and measurement of aggregation kinetics (i.e., rate of transition from a soluble oligomer to an insoluble aggregate). The HaloTag was attached to optoTAU, creating optoTAU-Halo, and two viscosity-sensitive AggFluor probes, P1h (soluble oligomers) and P18h (insoluble aggregates), were used that permit a dual color imaging platform to monitor the formation of soluble oligomers (P1h red; Ex/Em: 450/520 nm) and insoluble aggregates (P18h, green; Ex/Em: 540/640 nm) in live cells. Specifically, a stable, Dox-inducible optoTAU-Halo-expressing cell line was created for utilization with the “turn-on” AggFluor probes P1h and P18h. The light-selective biochemical detection of soluble HMW and insoluble optoTAU-Halo was detected (FIG. 15). Then, longitudinal live-cell time-lapse imaging of cells expressing optoTAU-Halo was performed after culturing the stable, Dox-inducible optoTAU-Halo-expressing cell line with doxycycline, P1h, P18h, and with or without W-MINK and incubated in the dark for 4 hours. Time lapse imaging was then performed in light conditions from 0 hours to 16 hours. A rapid increase in soluble tau oligomers (P1h fluorescence; FIG. 12A, top panel, FIG. 12B) was observed, which was followed by a progressive accumulation of insoluble tau aggregates (P18h fluorescence; FIG. 12B middle panel, FIG. 12C). Taken together, these results establish the first tau aggregation model that permits live-cell visualization of the temporal changes in tau solubility and aggregation.

### OptoTAU Forms Stable Irreversible Tau Aggregates Following Light Cessation

Given that the formation of insoluble tau aggregates is a physiologically irreversible process that persists throughout disease progression, we sought to determine whether light stimulation induces the irreversible formation of soluble HMW tau and insoluble tau aggregates following light cessation. Indeed, biochemical analysis indicates that soluble and insoluble optoTAU aggregates persist and are irreversible after light termination (FIG. 12E-I). Further immunocytochemical analysis reveals the progressive tau-driven formation of soluble and insoluble tau aggregates after light cessation (FIG. 12J-M). These results establish that light initiates the aggregation of optoTAU that persists over time in the absence of light.

### OptoTAU Reproduces Mutation- and Isoform-Specific Disease-Relevant Tau Aggregation Properties

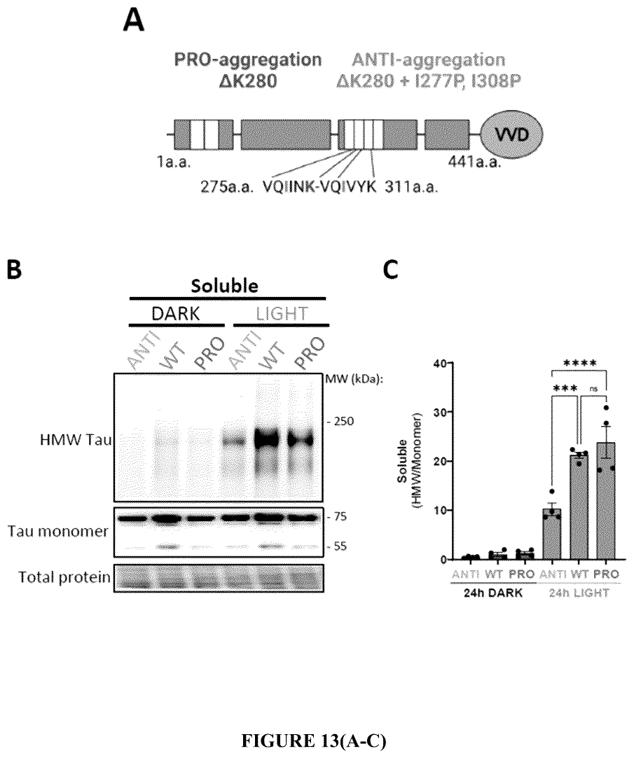

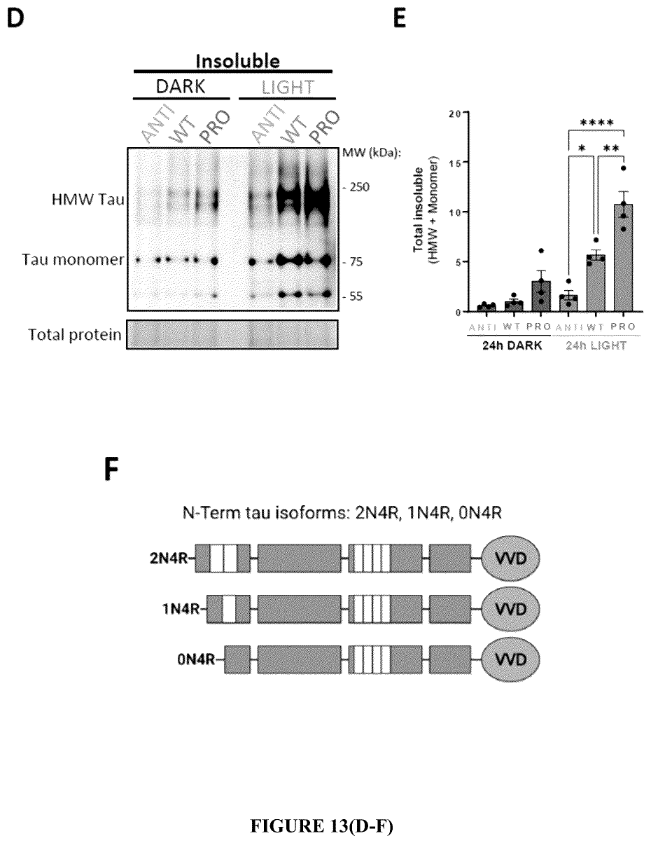

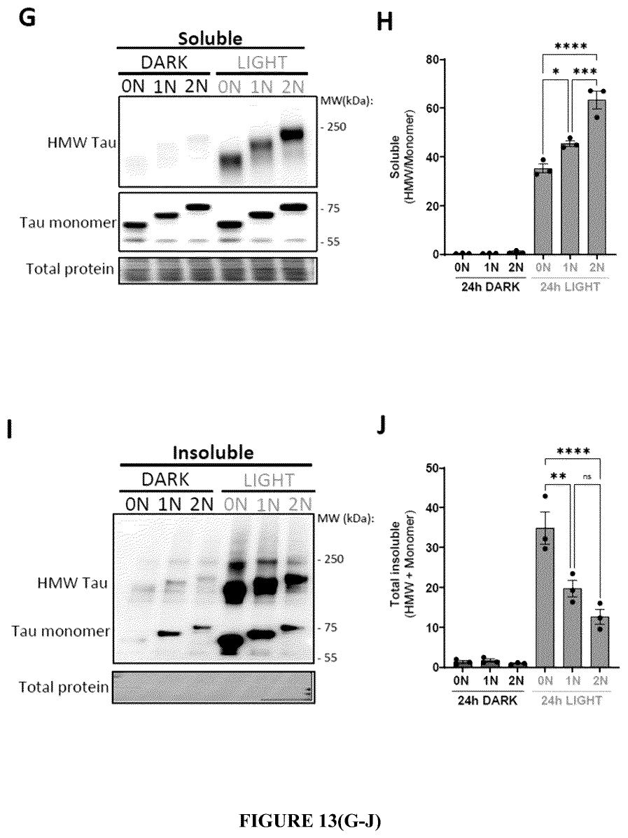

In familial tauopathies, mutations in the tau gene MAPT play a causal role in the accumulation of tau pathology. Two hexapeptide regions in the repeat domain of tau (275-280, 306-311) are critically involved in the formation of disease-specific tau conformations and the nucleation of tau aggregation. As such, we introduced a well-characterized, disease-causing, pro-aggregation mutation (\hat{ }K280; PRO) or an anti-aggregation mutation (\hat{ }K280, I277P, I308P; ANTI) into optoTAU (FIG. 13A). We show that the well-validated tau-specific pro-aggregation mutation (\hat{ }K280; PRO) does not affect the accumulation of soluble HMW tau (FIG. 13B, C) but dramatically increases the accumulation of insoluble optoTAU aggregation relative to optoTAU-WT (FIG. 13D, E). In contrast, the anti-aggregation (\hat{ }K280, I277P, I308P; ANTI) mutations lead to the accumulation of lower levels of soluble HMW tau and insoluble tau aggregates relative to optoTAU-WT (FIG. 13B-E). These results are consistent with aggregation propensities of pro- and anti-aggregation MAPT mutations.

Tau is expressed in six different isoforms in the adult human brain. Tau isoforms differ in the number of amino (N)-terminal inserts (0N, 1N, 2N) and contain either three or four 31-32-amino acid repeat sequences (R1-R4). NFTs in progressive supranuclear palsy predominantly contain 4R-tau and Pick disease inclusions contain mostly 3R-tau, whereas NFTs in AD contain a mixture of 4R-tau and 3R-tau, suggesting that splicing is of key importance in the neuropathological process in different tauopathies and that potentially cell-specific tau isoforms assume distinct physiological roles. In sporadic tauopathies, such as AD, different tau isoforms dictate aggregation propensity (i.e., 2N4R<1N4R<0N4R). Accordingly, we show that optoTAU isoforms exhibit a similar pattern of insoluble optoTAU accumulation as observed in human AD brains with a corresponding depletion in soluble HMW tau (FIG. 13F-J). These findings indicate that light-induced optoTAU aggregation is a faithful model of disease-causing mutation and isoform-specific aggregation properties of tau. Additionally, these results establish that while light initiates optoTAU aggregation, this process is subsequently driven by the distinguishing features of MAPT mutations and tau isoforms.

### Human Neurons Form OptoTAU Aggregates That Seed the Aggregation of Endogenous Tau and Form Amyloid-Like β-Sheet Structures

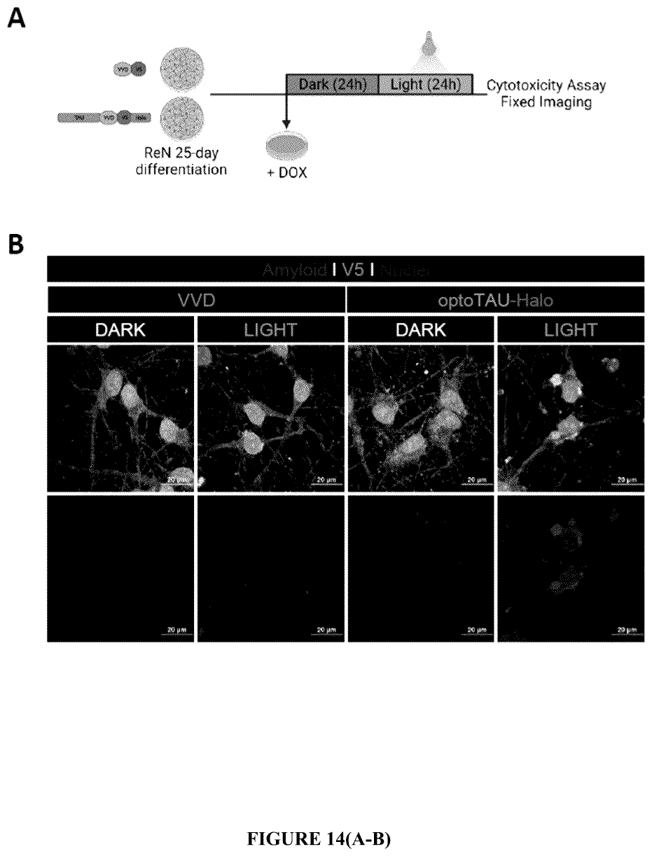

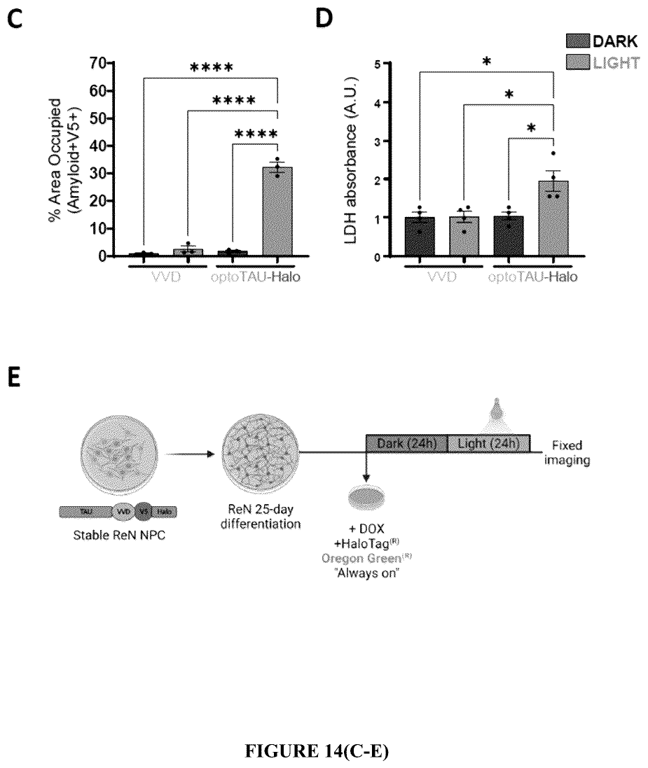

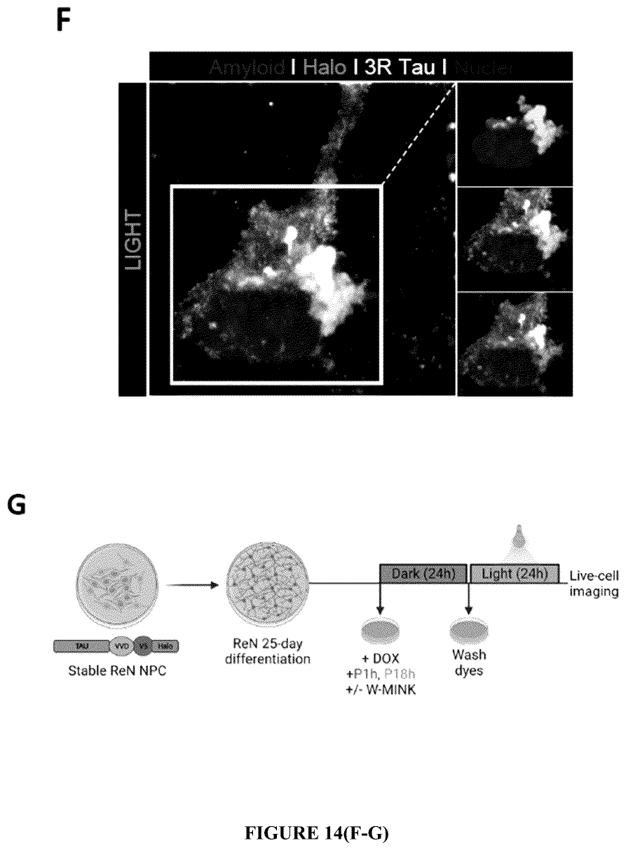

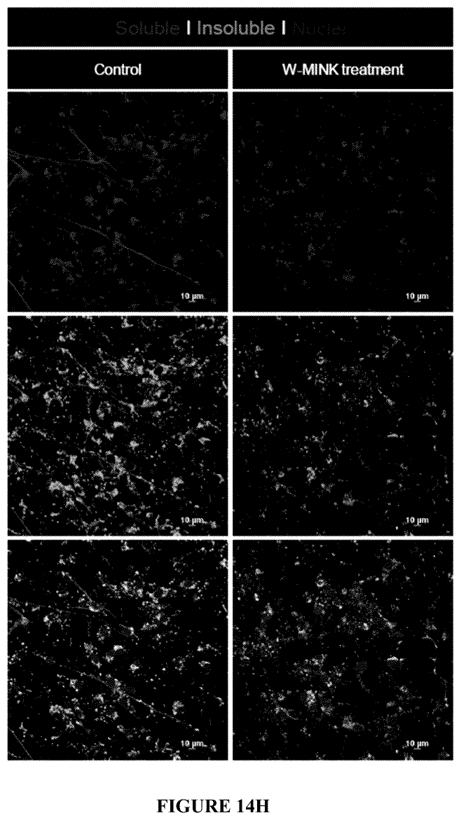

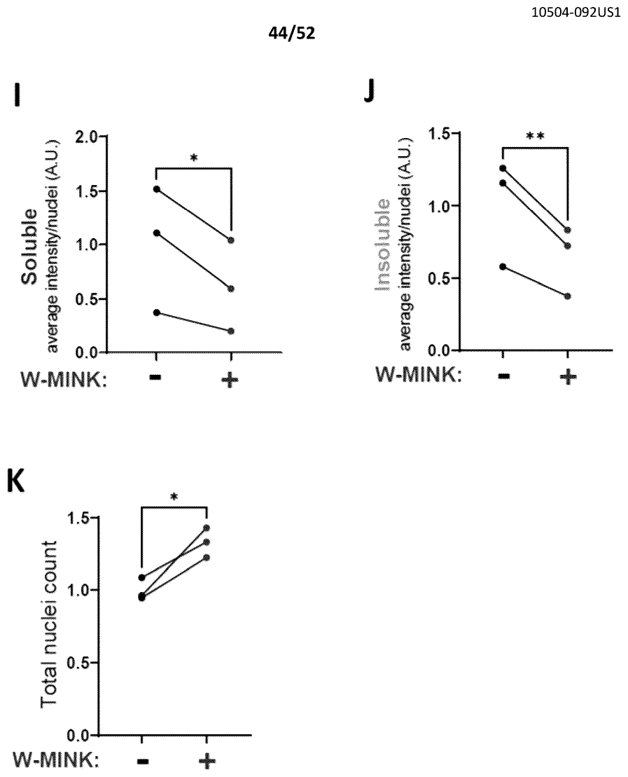

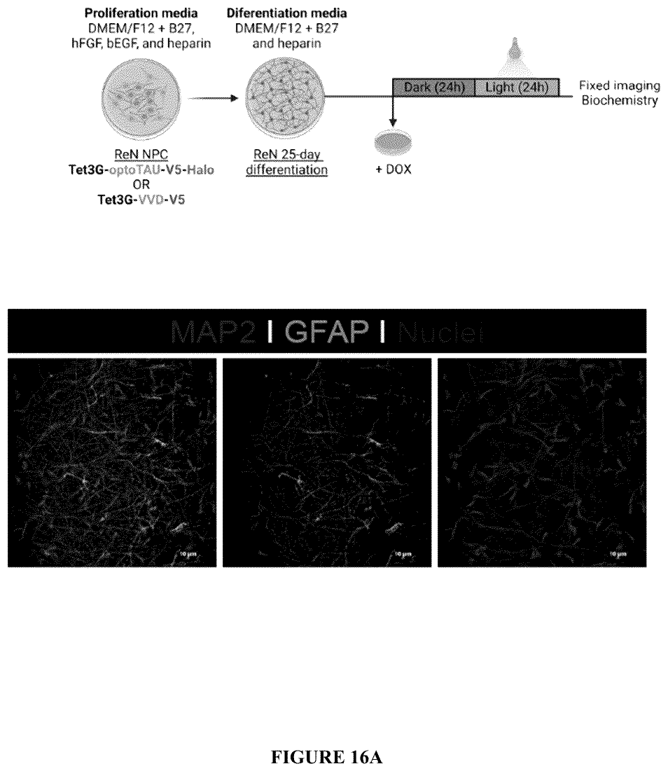

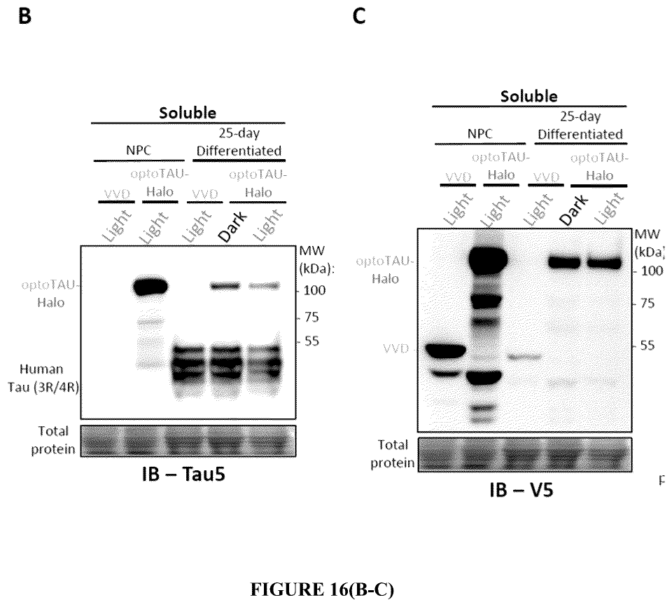

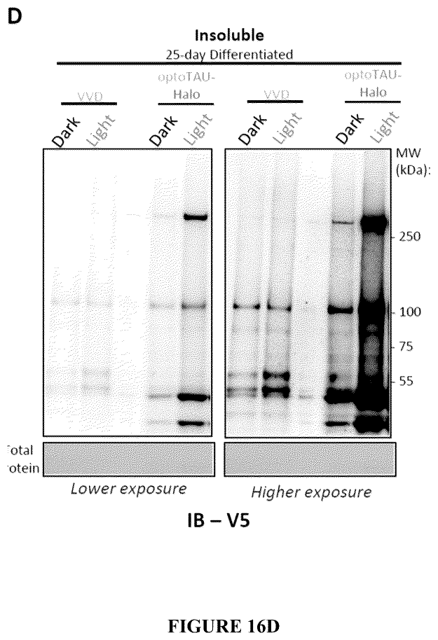

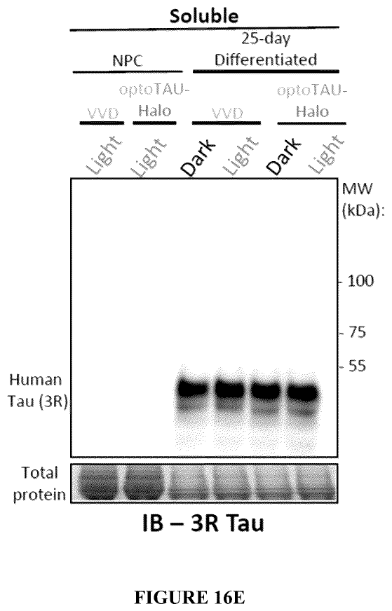

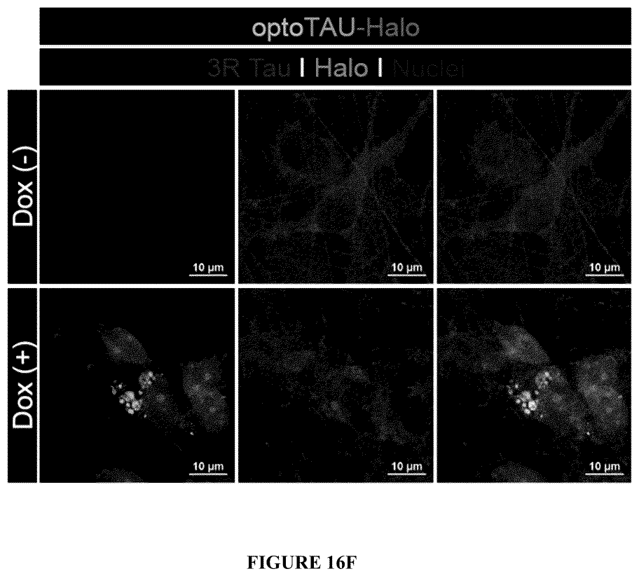

Insoluble tau aggregates deposit in amyloid-like β-sheet structures in neurons of AD patients and assume “prion-like” behavior, capable of seeding tau monomers into insoluble aggregates. To determine whether optoTAU-Halo is a model for tau self-assembly in human neurons, we used the human neural progenitor cell (NPC) line, RENCELL® VM, an established platform for modeling AD pathogenesis. First, we generated stable, Dox-inducible VVD-V5 (control) and optoTAU-Halo-expressing RENCELL® NPC lines. We then differentiated the NPCs into cortical-like (MAP2+, 3R and 209 4R Tau+) mature neurons (FIG. 16A) and biochemically established the light-selective detection of soluble and insoluble optoTAU-Halo in neurons (FIG. 16B-D). We then utilized the amyloid-sensitive dye, Amytracker, to show the formation of amyloid-positive optoTAU-Halo aggregates (FIG. 14A-C), which is associated with reduced neuronal viability (FIG. 14D). Taking advantage of the fact that ReNcell neurons express both 3R and 4R tau (40), we used a 3R tau isoform-specific antibody that does not detect optoTAU-Halo (2N4R) (FIG. 16E) to show that optoTAU-Halo recruits endogenous 3R tau into insoluble amyloid-positive tau aggregates after light exposure (FIG. 14E, F and FIG. 12F). Together, these results demonstrate that optoTAU-Halo adopts a biologically relevant amyloid structure and is a model of endogenous tau seeding in human neurons.

### A Tau Inhibitor Prevents Rescues Neuronal Viability Induced by OptoTAU Aggregation

The formation of tau aggregates leads to a decrease in neuronal survival (FIG. 14D). As such, we sought to determine whether we could rescue the reduced neuronal viability with a tau structure-based peptide inhibitor, W-MINK. First, using live-cell imaging (FIG. 14D), we observe a significant accumulation of soluble tau oligomers and insoluble tau aggregates in optoTAU-Halo-expressing neurons (FIG. 14H). Significantly, W-MINK treatment lowers levels of soluble oligomers and insoluble aggregates of optoTAU-Halo (FIG. 14H-J). Furthermore, W-MINK treatment prevents neuronal loss induced by optoTAU-Halo aggregation (FIG. 14K). Collectively, these results demonstrate that the AggFluor probes permit direct, live-cell visualization of temporal changes in tau solubility and aggregation in neurons and, importantly, provides a screening platform to test tau-specific aggregation inhibitors.

### Example 4

### Discussion

Post-mortem neuropathology studies and cross-sectional and longitudinal positron emission tomography (PET) imaging reveal the stepwise accumulation of Aβ and tau pathology but preclude a mechanistic understanding of how tau aggregation dictates disease progression in AD. Efforts to translate these findings into cellular and murine models of tau dysfunction have also been challenging due to the use of overexpressed mutated MAPT and species-specific differences. Furthermore, although several tau-targeted therapies have successfully prevented tau aggregation and propagation and promoted pathological tau clearance in pre-clinical models, most tau-targeted AD clinical trials have failed due to insufficient efficacy. As such, it is crucial to identify alternative targetable mechanisms that modify tau aggregation in biologically relevant human pre-clinical models, which may facilitate and increase the efficacy of human trials.

Optogenetics has revolutionized the field of protein activation, protein-protein interaction, and protein aggregation. It has become an invaluable technique to model neurodegenerative proteinopathies such as ALS, PD, and AD. To address the research challenges associated with investigating tau aggregation, we developed an optogenetic platform, termed optoTAU, to model tau aggregation in vitro. OptoTAU faithfully reproduces the biological and structural properties of tau aggregation observed in the human brain. We further developed optoTAU-Halo to visualize the multi-step transition in tau solubility during aggregation given that understanding the relationship between protein solubility and its role in physiological or pathological processes is also a significant research challenge. Finally, we provide proof-of-concept that optoTAU-Halo and the AggFluor technology provide a powerful scalable platform to investigate modifiers of tau pathobiology, model the consequences of tau aggregation, and serve as a drug discovery platform for modifiers of tau solubility and aggregation. Broadly, these tools may be applied to other aggregation-prone proteins and used to characterize complex relationships between protein solubility, cellular function, and disease progression.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A nucleotide sequence encoding a chimeric polypeptide, comprising: a first nucleotide sequence encoding a light-induced oligomerization domain and a second nucleotide sequence encoding a low complexity domain from a neurodegenerative disease target protein, wherein the low complexity domain from a neurodegenerative disease target protein is a Tau.

2. The nucleotide sequence of claim 1, wherein the first nucleotide sequence encodes an amino acid sequence that is at least 85% identical to SEQ ID NO:5.

3. The nucleotide sequence of claim 1, wherein the first nucleotide sequence encodes an amino acid sequence that is at least 90% identical to SEQ ID NO:5.

4. The nucleotide sequence of claim 1, wherein the first nucleotide sequence encodes an amino acid sequence that is at least 95% identical to SEQ ID NO:5.

5. The nucleotide sequence of claim 1, wherein the first nucleotide sequence encodes an amino acid sequence comprising SEQ ID NO:103.

6. The nucleotide sequence of claim 1, wherein the Tau comprises SEQ ID NO:104.

7. The nucleotide sequence of claim 1, further comprising a third nucleotide sequence encoding a visualization probe binding sequence.

8. The polynucleotide sequence of claim 7, wherein the visualization probe is a fluorescent dye.

9. The polynucleotide sequence of claim 8, wherein the visualization probe binding sequence is a modified haloalkane dehalogenase.

10. A method of inducing a neurodegenerative disease pathology in a cell, comprising the steps:
a. introducing into the cell an expression vector encoding a chimeric polypeptide, comprising:
b. a first nucleotide sequence encoding a light-induced oligomerization domain and a second nucleotide sequence encoding a low complexity domain from a neurodegenerative disease target protein, wherein the first nucleotide sequence is operably linked to a promoter and wherein the low complexity domain from a neurodegenerative disease target protein is a Tau;
c. expressing the chimeric polypeptide; and
d. inducing oligomerization of the chimeric polypeptide by stimulation with blue light.

11. The method of claim 10, wherein the first nucleotide sequence encodes an amino acid sequence that is at least 85% identical to SEQ ID NO:5.

12. The method of claim 10, wherein the first nucleotide sequence encodes an amino acid sequence that is at least 90% identical to SEQ ID NO:5.

13. The method of claim 10, wherein the first nucleotide sequence encodes an amino acid sequence that is at least 95% identical to SEQ ID NO:5.

14. The method of claim 10, wherein the first nucleotide sequence encodes an amino acid sequence comprising SEQ ID NO:103.

15. The method of claim 10, wherein the Tau comprises SEQ ID NO:104.

16. The method of claim 10, wherein the expression vector further comprises a third nucleotide sequence encoding a visualization probe binding sequence.

17. The method of claim 16, wherein the visualization probe is a fluorescent dye.

18. The method of claim 17, wherein the visualization probe binding sequence is a modified haloalkane dehalogenase.

19. A method of screening for an agent that modulates protein aggregation or solubility, comprising the steps:
a. introducing into a cell an expression vector encoding a chimeric polypeptide, comprising a first nucleotide sequence encoding a light-induced oligomerization domain and a second nucleotide sequence encoding a low complexity domain from a neurodegenerative disease target protein, wherein the first nucleotide sequence is operably linked to a promoter and wherein the low complexity domain from a neurodegenerative disease target protein is a Tau;
b. expressing the chimeric polypeptide;
c. introducing the agent into a culture media comprising the cell;
d. inducing oligomerization of the chimeric polypeptide by stimulation with blue light; and
e. determining modulation of protein aggregation or solubility by the agent.

20. The method of claim 19, wherein the first nucleotide sequence encodes an amino acid sequence that is at least 85% identical to SEQ ID NO:5.

21. The method of claim 19, wherein the first nucleotide sequence encodes an amino acid sequence that is at least 90% identical to SEQ ID NO:5.

22. The method of claim 19, wherein the first nucleotide sequence encodes an amino acid sequence that is at least 95% identical to SEQ ID NO:5.

23. The method of claim 19, wherein the first nucleotide sequence encodes an amino acid sequence comprising SEQ ID NO:103.

24. The method of claim 19, wherein the Tau comprises SEQ ID NO:104.

25. The method of claim 19, wherein the expression vector further comprises a third nucleotide sequence encoding a visualization probe binding sequence.

26. The method of claim 25, wherein the visualization probe is a fluorescent dye.

27. The method of claim 26, wherein the visualization probe binding sequence is a modified haloalkane dehalogenase.


SEQUENCE LISTING (106 sequences)

SEQ ID NO: 1 - protein, 498 aa, synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 2 - protein, 498 aa, Arabidopsis thaliana
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 3 - protein, 119 aa, Neurospora crassa
MTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 4 - protein, 150 aa, Neurospora crassa
HTLYAPGGYDIMGYLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 5 - protein, 150 aa, Neurospora crassa
HTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 6 - protein, 915 aa, synthetic construct
  REGION 1..915: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 7 - protein, 916 aa, synthetic construct
  REGION 1..916: note = synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 8 - protein, 644 aa, synthetic construct
  REGION 1..644: note = synthetic construct
MGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 9 - protein, 727 aa, synthetic construct
  REGION 1..727: note = synthetic construct
MRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 10 - protein, 813 aa, synthetic construct
  REGION 1..813: note = synthetic construct
MDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 11 - protein, 642 aa, synthetic construct
  REGION 1..642: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 12 - protein, 725 aa, synthetic construct
  REGION 1..725: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 13 - protein, 811 aa, synthetic construct
  REGION 1..811: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 14 - protein, 1159 aa, synthetic construct
  REGION 1..1159: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMSRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 15 - protein, 1160 aa, synthetic construct
  REGION 1..1160: note = synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 16 - protein, 1152 aa, synthetic construct
  REGION 1..1152: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 17 - protein, 888 aa, synthetic construct
  REGION 1..888: note = synthetic construct
MGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 18 - protein, 971 aa, synthetic construct
  REGION 1..971: note = synthetic construct
MRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 19 - protein, 1057 aa, synthetic construct
  REGION 1..1057: note = synthetic construct
MDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 20 - protein, 878 aa, synthetic construct
  REGION 1..878: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 21 - protein, 961 aa, synthetic construct
  REGION 1..961: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 22 - protein, 1047 aa, synthetic construct
  REGION 1..1047: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 23 - protein, 886 aa, synthetic construct
  REGION 1..886: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMSRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 24 - protein, 969 aa, synthetic construct
  REGION 1..969: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMSRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 25 - protein, 1057 aa, synthetic construct
  REGION 1..1057: note = synthetic construct
MDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAASRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 26 - protein, 566 aa, synthetic construct
  REGION 1..566: note = synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEAMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 27 - protein, 569 aa, synthetic construct
  REGION 1..569: note = synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 28 - protein, 466 aa, synthetic construct
  REGION 1..466: note = synthetic construct
MDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 29 - protein, 380 aa, synthetic construct
  REGION 1..380: note = synthetic construct
MRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 30 - protein, 297 aa, synthetic construct
  REGION 1..297: note = synthetic construct
MGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 31 - protein, 462 aa, synthetic construct
  REGION 1..462: note = synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEADLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 32 - protein, 376 aa, synthetic construct
  REGION 1..376: note = synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEARKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 33 - protein, 292 aa, synthetic construct
  REGION 1..292: note = synthetic construct
HTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEAGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 34 - protein, 808 aa, synthetic construct
  REGION 1..808: note = synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEAMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFAPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 35 - protein, 809 aa, synthetic construct
  REGION 1..809: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLKMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 36 - protein, 705 aa, synthetic construct
  REGION 1..705: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLKDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 37 - protein, 619 aa, synthetic construct
  REGION 1..619: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLKRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 38 - protein, 536 aa, synthetic construct
  REGION 1..536: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLKGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 39 - protein, 704 aa, synthetic construct
  REGION 1..704: note = synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEADLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFAPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 40 - protein, 618 aa, synthetic construct
  REGION 1..618: note = synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEARKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFAPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 41 - protein, 535 aa, synthetic construct
  REGION 1..535: note = synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEAGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMFAPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 42 - protein, 639 aa, synthetic construct
  REGION 1..639: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 43 - protein, 644 aa, synthetic construct
  REGION 1..644: note = synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEADPPVATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAA

SEQ ID NO: 44 - protein, 881 aa, synthetic construct
  REGION 1..881: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEADPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 45 - protein, 882 aa, synthetic construct
  REGION 1..882: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 46 - protein, 881 aa, synthetic construct
  REGION 1..881: note = synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEADPPVATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 47 - protein, 291 aa, synthetic construct
  REGION 1..291: note = synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAFHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 48 - protein, 292 aa, synthetic construct
  REGION 1..292: note = synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEAMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 49 - protein, 528 aa, synthetic construct
  REGION 1..528: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYSKMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAFHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 50 - protein, 547 aa, synthetic construct
  REGION 1..547: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEALEFCSRRYRGPMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 51 - protein, 534 aa, synthetic construct
  REGION 1..534: note = synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEAMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAFAPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 52 - protein, 558 aa, synthetic construct
  REGION 1..558: note = synthetic construct
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEALLPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASNSAVDGTAGPMHTLYAPGGYDIMGYLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGIHRI

SEQ ID NO: 53 - protein, 551 aa, synthetic construct
  REGION 1..551: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEASNSAVDGTAGPMHTLYAPGGYDIMGYLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGIHRI

SEQ ID NO: 54 - protein, 551 aa, synthetic construct
  REGION 1..551: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASNSAVDGTAGPMHTLYAPGGYDIMGYLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGMDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAHRI

SEQ ID NO: 55 - protein, 702 aa, synthetic construct
  REGION 1..702: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASNSAVDGTAGPMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLGIHRI

SEQ ID NO: 56 - protein, 879 aa, synthetic construct
  REGION 1..879: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASNSAVDGTAGPMPDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRKPGGIHRIMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLITDHNQPYHICRGFTCFKKPPTPPPEPET

SEQ ID NO: 57 - protein, 852 aa, synthetic construct
  REGION 1..852: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASNSAVDGTAGPMHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEGMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLHRI

SEQ ID NO: 58 - protein, 853 aa, synthetic construct
  REGION 1..853: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLTMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLRRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEALEFCSRRYRGPGIHRI

SEQ ID NO: 59 - protein, 853 aa, synthetic construct
  REGION 1..853: note = synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEALAMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLRCSRRYRGPGIHRI

SEQ ID NO: 60 - protein, 859 aa, synthetic construct
  REGION 1..859: note = synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLALPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQRHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETEALEFCSRRYRGPGIHRI

SEQ ID NO: 61 - protein, 1190 aa, synthetic construct
  REGION 1..1190: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSRMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLGDSRS

SEQ ID NO: 62 - protein, 706 aa, synthetic construct
  REGION 1..706: note = synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLFATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGLRSRAQASNSAVDGTAGPGSTGSR

SEQ ID NO: 63 - protein, 1193 aa, synthetic construct
  REGION 1..1193: note = synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLALPDSDLEATMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 64 - protein, 1184 aa, synthetic construct
  REGION 1..1184: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAPMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLGDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 65 - protein, 1190 aa, synthetic construct; identical to SEQ ID NO: 61
  REGION 1..1190: note = synthetic construct

SEQ ID NO: 66 - protein, 853 aa, synthetic construct; identical to SEQ ID NO: 58
  REGION 1..853: note = synthetic construct

SEQ ID NO: 67 - protein, 853 aa, synthetic construct; identical to SEQ ID NO: 59
  REGION 1..853: note = synthetic construct

SEQ ID NO: 68 - protein, 971 aa, synthetic construct
  REGION 1..971: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLATLDHNQPYHICRGFTCFKKPPTPPPEPET

SEQ ID NO: 69 - protein, 583 aa, synthetic construct
  REGION 1..583: note = synthetic construct
MHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLHRI

SEQ ID NO: 70 - protein, 969 aa, synthetic construct
  REGION 1..969: note = synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLMKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAATLDHNQPYHICRGFTCFKKPPTPPPEPET

SEQ ID NO: 71 - protein, 604 aa, synthetic construct
  REGION 1..604: note = synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLPDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRKPGLQSTVPRARDPPDLDN

SEQ ID NO: 72 - protein, 607 aa, synthetic construct
  REGION 1..607: note = synthetic construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLHTLYAPGGYDIMGWLIQIMNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETELQSTVPRARDPPDLDN

SEQ ID NO: 73 - protein, 607 aa, synthetic construct; identical to SEQ ID NO: 72
  REGION 1..607: note = synthetic construct

SEQ ID NO: 74 - protein, 608 aa, synthetic construct
  REGION 1..608: note = synthetic construct
MPDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRKPGSLMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLSNSAVDGTAGPGSTGSR

SEQ ID NO: 75 - protein, 1159 aa, synthetic construct
  REGION 1..1159: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGLGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMSRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 76 - protein, 1159 aa, synthetic construct
  REGION 1..1159: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAARGMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRALAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMSRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 77 - protein, 1157 aa, synthetic construct
  REGION 1..1157: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKAAGWGMWDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 78 - protein, 1157 aa, synthetic construct
  REGION 1..1157: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKDDGWGMWDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 79 - protein, 1156 aa, synthetic construct
  REGION 1..1156: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPVMMAAAQAALQSSWGMMGMLASQQNQSGPSGNNQNQGNMQREPNQAFGSGNNSYSGSNSGAAIGWGSASNAGSGSGFNGGFGSSMDSKSSGWGWDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 80 - protein, 1156 aa, synthetic construct
  REGION 1..1156: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMVGMLASQQNQSGPSGNNQNQGNMQREPNQAFGSGNNSYSGSNSGAAIGWGSASNAGSGSGFNGGFGSSMDSKSSGWGWDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 81 - protein, 884 aa, synthetic construct
  REGION 1..884: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQGRFGGNPGGFGNQGGFGNSRGGGAGLGNNQGSNMGGGMNFGAFSINPVMMAAAQAALQSSWGMMGMLASQQNQSGPSGNNQNQGNMQREPNQAFGSGNNSYSGSNSGAAIGWGSASNAGSGSGFNGGFGSSMDSKSSGWGMRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 82 - protein, 884 aa, synthetic construct
  REGION 1..884: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQGRFGGNPGGFGNQGGFGNSRGGGAGLGNNQGSNMGGGMNFGAFSINPAMMAAAQAALQSSWGMVGMLASQQNQSGPSGNNQNQGNMQREPNQAFGSGNNSYSGSNSGAAIGWGSASNAGSGSGFNGGFGSSMDSKSSGWGMRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 83 - protein, 884 aa, synthetic construct
  REGION 1..884: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKAAGWGMRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 84 - protein, 885 aa, synthetic construct
  REGION 1..885: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAPGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKDDGWGMTRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 85 - protein, 1157 aa, synthetic construct
  REGION 1..1157: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGLGLVRFTEYETQVKVMSQRHMIDGRWCDCKLPNSKQSQDEPLRSRKVLVGRCTEDMTEDELREFFSQYGDVMDVFIPKPFRALALVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGMWDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 86 - protein, 829 aa, synthetic construct
  REGION 1..829: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQDLIVLGLPWKTTEQDLKEYFSTFGEVLMVQVKKDLKTGHSKGFGFVRFTEYETQVKVMSQRHMIDGRWCDCKLPNSKQSQDEPLRSRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 87 - protein, 815 aa, synthetic construct
  REGION 1..815: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQRKVFVGRCTEDMTEDELREFFSQYGDVMDVFIPKPFRAFAFVTFADDQIAQSLCGEDLIIKGISVHISNAEPRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 88 - protein, 901 aa, synthetic construct
  REGION 1..901: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 89 - protein, 1016 aa, synthetic construct
  REGION 1..1016: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQMSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSRDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 90 - protein, 1040 aa, synthetic construct
  REGION 1..1040: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQCRATMYPYDVPDYAMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 91 - protein, 1283 aa, synthetic construct
  REGION 1..1283: note = synthetic construct
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTRGAQIMIGAAAQCRATMYPYDVPDYAMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPYWDPPVATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 92 - protein, 348 aa, synthetic construct
  REGION 1..348: note = synthetic construct
MNGLTPPLMFCSRSDDPSSTSNINLDDVFADVFFNSNGELLDIDEIDDFGDNTCPKSSMSVDDDASSQVFQGHLFGNALSSIALSDSGDLSTGIYESQGNASRGKSLRTKSSGSISSELTEAQKVERRERNREHAKRSRVRKKFLLESLQQSVNELNHENNCLKESIREHLGPRGDSLIAQCSPEADTLLTDNPSKANRILEDPDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRK

SEQ ID NO: 93 - protein, 145 aa, synthetic construct
  REGION 1..145: note = synthetic construct
PDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRK

SEQ ID NO: 94 - DNA, 435 bp, synthetic construct
  misc_feature 1..435: note = synthetic construct
  source 1..435: mol_type = other DNA
cctgactacagtctcgtgaaggctctgcaaatggcacaacagaattttgtcattacagacgcctccctcccagacaaccctatcgtctacgccagtagagggtttctgacactgacaggctattctctcgaccagatcctgggcaggaactgcaggtttctgcaagggccagaaacagacccaagagctgtggataagatcaggaatgccatcaccaaaggcgttgataccagtgtctgtctgctgaattatagacaggatggcacaaccttctggaatctcttcttcgtggctggactcagagattctaagggcaatattgtcaactacgtcggagtgcagtcaaaggtgagcgaagattatgccaagctgctggtcaacgagcagaacattgagtacaaaggtgtgcgcaccagtaacatgctgcgcagaaag

SEQ ID NO: 95 - protein, 414 aa, synthetic construct
  REGION 1..414: note = synthetic construct
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 96 - protein, 311 aa, synthetic construct
  REGION 1..311: note = synthetic construct
MDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 97 - protein, 225 aa, synthetic construct
  REGION 1..225: note = synthetic construct
MRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 98 - protein, 142 aa, synthetic construct
  REGION 1..142: note = synthetic construct
MGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 99 - protein, 161 aa, synthetic construct
  REGION 1..161: note = synthetic construct
GEFLATTLERIEKNFVITDPRLPDNPIIFASDSFLQLTEYSREEILGRNCRFLQGPETDRATVRKIRDAIDNQTEVTVQLINYTKSGKKFWNLFHLQPMRDQKGDVQYFIGVQLDGTEHVRDAAEREGVMLIKKTAENIDEAAKELPDANLRPEDLWANHG

SEQ ID NO: 100 - protein, 225 aa, synthetic construct
  REGION 1..225: note = synthetic construct
MLDMGQDRPIDGSGAPGADDTRVEVQPPAQWVLDLIEASPIASVVSDPRLADNPLIAINQAFTDLTGYSEEECVGRNCRFLAGSGTEPWLTDKIRQGVREHKPVLVEILNYKKDGTPFRNAVLVAPIYDDDDELLYFLGSQVEVDDDQPNMGMARRERAAEMLKTLSPRQLEVTTLVASGLRNKEVAARLGLSEKTVKMHRGLVMEKLNLKTSADLVRIAVEAGI

SEQ ID NO: 101 - protein, 261 aa, synthetic construct
  REGION 1..261: note = synthetic construct
MASFQSFGIPGQLEVIKKALDHVRVGVVITDPALEDNPIVYVNQGFVQMTGYETEEILGKNCRFLQGKHTDPAEVDNIRTALQNKEPVTVQIQNYKKDGTMFWNELNIDPMEIEDKTYFVGIQNDITKQKEYEKLLEDSLTEITALSTPIVPIRNGISALPLVGNLTEERFNSIVCTLTNILSTSKDDYLIIDLSGLAQVNEQTADQIFKLSHLLKLTGTELIITGIKPELAMKMNKLDANFSSLKTYSNVKDAVKVLPIM

SEQ ID NO: 102 - protein, 176 aa, synthetic construct
  REGION 1..176: note = synthetic construct
MDQKQFEKIRAVFDRSGVALTLVDMSLPEQPVVLANPPFLRMTGYTEGQILGFNCRFLQRGDENAQARADIRDALKLGRELQVVLRNYRANDEPFDNLLFLHPVGGRPDAPDYFLGSQFELGRSGNSEEAAAAGHAGALTGELARIGTVAARLEMDSRRHLAQAAAALVRAWERRG

SEQ ID NO: 103 - protein, 156 aa, synthetic construct
HTLYAPGGYDIMGWLIQIMNRPNPQVELGPVDTSCALVLCDLKQKDTPVVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETECFKLGT

SEQ ID NO: 104 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 105 - protein, 185 aa, Neurospora crassa
MSHTVNSSTMNPWEVEAYQQYHYDPRTAPTANPLFFHTLYAPGGYDIMGYLIQIMNRPNPQVELGPVTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 106 - protein, 10 aa, synthetic construct
DVWMINKKRK